(19) United States
(12) Patent Application Publication
(10) Pub. No.: US 2024/0412815 A1
(43) Pub. Date: Dec. 12, 2024

(54) DETECTING HOMOLOGOUS RECOMBINATION DEFICIENCES BASED ON METHYLATION STATUS OF CELL-FREE NUCLEIC ACID MOLECULES

(71) Applicant: GUARDANT HEALTH, INC., Palo Alto, CA (US)

(72) Inventors: Catalin BARBACIORU, Fremont, CA (US); Daniel GAILE, Buffalo, NY (US)

(21) Appl. No.: 18/545,747

(22) Filed: Dec. 19, 2023

Related U.S. Application Data
(60) Provisional application No. 63/476,614, filed on Dec. 21, 2022.

(51) Int. Cl. G16B 20/20; G06N 5/022; G16B 40/20

(52) U.S. Cl. CPC G16B 20/20; G06N 5/022; G16B 40/20

(57) ABSTRACT

In implementations described herein, methylation information is determined with respect to classification regions of a reference genome that are related to the presence of a homologous recombination repair deficiency in a subject. The methylation information can be analyzed using a number of computational techniques to provide metrics related to the presence or absence of a homologous recombination repair deficiency in a given subject.

### CROSS-REFERENCE TO RELATED APPLICATIONS

This application claims the benefit of U.S. Provisional Application No. 63/476,614, filed on Dec. 21, 2022, which is incorporated herein by reference for all purposes.

### BACKGROUND

Cancer is a major cause of disease worldwide. Each year, tens of millions of people are diagnosed with cancer around the world, and more than half eventually die from it. In many countries, cancer ranks the second most common cause of death following cardiovascular diseases. Early detection is associated with improved outcomes for many cancers.

Cancer can be caused by the accumulation of genetics variations within an individual's normal cells, at least some of which result in improperly regulated cell division. Such variations commonly include copy number variations (CNVs), single nucleotide variations (SNVs), gene fusions, insertions and/or deletions (indels), epigenetic variations including 5-methylation of cytosine (5-methylcytosine) and association of DNA with chromatin and transcription factors.

Cancers are often detected by biopsies of tumors followed by analysis of cells, markers or DNA extracted from cells. But more recently it has been proposed that cancers can also be detected from cell-free nucleic acids in body fluids, such as blood or urine. Such tests have the advantage that they are noninvasive and can be performed without identifying suspected cancer cells in biopsy. However, such tests are complicated by the fact that the amount of nucleic acids in body fluids is very low and what nucleic acids are present are heterogeneous in form (e.g., RNA and DNA, single-stranded and double-stranded, and various states of post-replication modification and association with proteins, such as histones).

Thus, there is a need for improved systems and methods for improved cancer detection using liquid biopsy assays. Therefore, it is an object of the disclosure to provide computer-implemented systems and methods that have improved capability to classify a sample as containing tumor-derived DNA with heightened sensitivity.

### BRIEF DESCRIPTION OF THE DRAWINGS

The accompanying drawings, which are incorporated in and constitute a part of this specification, illustrate certain implementations, and together with the written description, serve to explain certain principles of the methods, computer readable media, and systems disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 is a diagrammatic representation of an example architecture that partitions nucleic acids based on methylation of cytosines in one or more genomic regions of a reference sequence, according to one or more implementations.

FIG. 2 is a diagrammatic representation of an example framework to determine homologous recombination deficiency status using one or more computational models and based on methylation status of cell free nucleic acid molecules, according to one or more implementations.

FIG. 3 is a diagrammatic representation of an example framework to generate a computational model to determine homologous recombination deficiency status of a subject, in accordance with one or more implementations.

FIG. 4 is a flow diagram of an example process to determine a probability of a homologous recombination repair deficiency being present in one or more subjects based on methylation data derived from a sample obtained from the one or more subjects, according to one or more implementations.

FIG. 5 is a block diagram illustrating components of a machine, in the form of a computer system, that may read and execute instructions from one or more machine-readable media to perform any one or more methodologies described herein, in accordance with one or more example implementations.

FIG. 6 is block diagram illustrating a representative software architecture that may be used in conjunction with one or more hardware architectures described herein, in accordance with one or more example implementations.

FIG. 7 is a chart showing genomic regions identified as predictor regions for homologous recombination repair deficiencies using techniques described herein.

FIG. 8 includes a number of graphs showing probit values related to determinations of subjects being positive for a homologous repair deficiency (HRD) or negative for an HRD using techniques described herein for a number of forms of cancer.

### SUMMARY

In one aspect, a method includes obtaining, by a computing system having one or more hardware processors and memory, training sequence data including training sequence representations derived from a plurality of samples, individual training sequence representations including a nucleotide sequence corresponding to a fragment of a nucleic acid included in a sample of a plurality of samples and individual samples of the plurality of samples corresponding to a subject classified as having a homologous recombination repair deficiency. The method also includes determining, by the computing system, a subset of the training sequence representations that correspond to nucleic acids having at least a threshold amount of methylated cytosines in one or more regions of the nucleotide sequence. In addition, the method includes analyzing, by the computing system, the subset of training sequence representations to determine quantitative measures derived from the subset of the training sequence representations, individual quantitative measures corresponding to a classification region of a plurality of classification regions of a reference genome, individual classification regions of the plurality of classification regions having the threshold amount of methylated cytosines in subjects in which cancer is detected. Further, the method includes analyzing, by the computing system and using one or more computational techniques, the quantitative measures of the plurality of classification regions to determine a subset of the plurality of classification regions having at least a threshold likelihood of indicating a homology directed repair deficiency. The method includes generating, by the computing system, a predictive model to determine a probability of a homologous recombination repair deficiency being present in one or more additional subjects, the predictive model including a plurality of variables and a plurality of weights with individual weights of the plurality of weights corresponding to individual variables of the plurality of variables, where an individual variable of the plurality of variables corresponds to an individual classification region of the subset of the plurality of classification regions and an individual weight that corresponds to the individual variable indicates a likelihood of the individual classification region indicating a homologous recombination repair deficiency. The method also includes administering to the subject, a treatment suitable for treating homologous recombination repair deficiency based on the classification as having the homologous recombination repair deficiency.

In one or more examples, the method may include analyzing, by the computing system, the subset of training sequence representations to determine additional quantitative measures derived from the subset of the training sequence reads, individual quantitative measures corresponding to a control region of a plurality of control regions of a reference genome, individual control regions of the plurality of control regions having the threshold amount of methylated cytosines in subjects in which cancer is detected and in further subjects in which cancer is not detected, and determining, by the computing system, normalized quantitative measures that correspond to the subset of the plurality of classification regions, where an individual normalized quantitative measure is determined according to the quantitative measure that corresponds to a classification region of the subset of the plurality of classification regions and the additional quantitative measures.

In various examples, the method may include determining, by the computing system and implementing the predictive model, individual probabilities of a homologous recombination repair deficiency being present in individual samples of the plurality of samples based on the normalized quantitative measures corresponding to the individual samples, and determining, by the computing system and based on the individual probabilities, a threshold probability to indicate a homologous recombination repair deficiency being present with respect to a given subject.

In addition, the method may include determining, by the computing system, a responsiveness to treatment with respect to a group of subjects, where cancer is detected in the group of subjects and the treatment is provided to treat the cancer, and determining, by the computing system, the plurality of samples that correspond to subjects having a homologous recombination repair deficiency based on the responsiveness of a portion of the group of subjects to the treatment being at least a threshold level of responsiveness.

Further, the method may include analyzing, by the computing system, additional sequence reads derived from samples of a group of subjects in which cancer is detected to determine whether one or more genomic mutations are present with respect to one or more genomic regions, where the one or more genomic mutations correspond to homologous recombination repair pathways, and determining, by the computing system, the plurality of samples used to produce the training sequence representations by identifying a portion of the samples derived from the group of subjects in which the one or more genomic mutations are present.

In at least some examples, the one or more computational techniques include implementing one or more logistic regression models with elastic regularization.

In one or more additional examples, the method may include implementing, by the computing system, the predictive model to determine a probability of a homologous recombination repair deficiency being present in a plurality of additional samples, the plurality of additional samples being derived from additional subjects with a first form of cancer being detected in a first portion of the additional subjects and a second form of cancer being detected in a second portion of the additional subjects.

In one or more further examples, the method may include implementing, by the computing system, the predictive model to determine a probability of a homologous recombination repair deficiency being present in a plurality of additional samples, the plurality of additional samples being derived from additional subjects in which a single form of cancer is present.

In one or more examples, the method may include analyzing, by the computing system, the subset of training sequence reads to determine a group of training sequence reads that correspond to a plurality of genomic regions associated with homologous recombination repair pathways, and determining, by the computing system, one or more additional quantitative measures based on a number of the group of training sequence representations that correspond to at least a portion of the plurality of genomic regions,

Additionally, the plurality of classification regions may have at least a threshold amount of cytosine-guanine content.

The method may also include determining, by the computing system, tumor fraction estimates for a number of samples, the number of samples corresponding to subjects in which cancer is detected, analyzing, by the computing system, the tumor fraction estimates with respect to a threshold tumor fraction estimate, and determining, by the computing system, the plurality of samples used to derive the training sequence reads based on identifying at least a portion of the number of samples having a tumor fraction estimate corresponding to at least the threshold tumor fraction estimate.

In various examples, the method may include obtaining, by the computing system, testing sequence data from an additional subject that is not included in the plurality of subjects, the testing sequence data including testing sequencing representations derived from a sample of the additional subject, individual testing sequencing representations including a nucleotide sequence corresponding to a fragment of a nucleic acid included in the additional sample and individual testing sequencing reads corresponding to molecules having the threshold amount of methylated cytosines included in regions of the nucleotide, and determining, using the predictive model and the additional sequence data, a probability of a homologous recombination repair deficiency being present in the additional subject.

Further, the method may include combining a plurality of nucleic acids derived from at least one of blood or tissue of a subject with a solution including an amount of methyl binding domain (MBD) proteins to produce a nucleic acid-MBD protein solution, and performing a plurality of washes of the nucleic acid-MBD protein solution with a salt solution to produce a number of nucleic acid fractions, individual nucleic acid fractions having a threshold number of methylated cytosines in regions of the plurality of nucleic acids having at least a threshold cytosine-guanine content. Other technical features may be readily apparent to one skilled in the art from the following figures, descriptions, and claims.

In at least some examples, determining the subset of the plurality of classification regions having at least a threshold likelihood of indicating a homology directed repair deficiency may include determining, by the computing system and for individual classification regions of the plurality of classification regions, differences between a first portion of the normalized quantitative measures derived from samples that correspond to subjects in which a homology directed repair deficiency is present and a second portion of the normalized quantitative measures derived from samples that correspond to additional subjects in which a homologous recombination repair deficiency is not present, and determining, by the computing system, that an individual classification region is included in the subset of the plurality of classification regions based on the difference between the first portion of the normalized quantitative measures for the individual classification region and the second portion of the normalized quantitative measures for the individual classification region being at least a threshold difference.

In one or more additional examples, the treatment may include a poly adenosine diphosphate (ADP) ribose polymerase (PARP) inhibitor. The method also includes administering to the additional subject, a treatment suitable for treating homologous recombination repair deficiency based on the classification as having the homologous recombination repair deficiency.

In one or more further examples, the method may include determining, by the computing system, an additional subset of the training sequence representations that correspond to additional nucleic acids having less than an additional threshold amount of methylation, analyzing, by the computing system, the additional subset of the training sequence reads to determine an additional group of training sequence representations that correspond to the plurality of genomic regions associated with the homologous recombination repair pathways, determining, by the computing system, one or more further quantitative measures based on an additional number of the additional group of training sequence representations that correspond to at least a portion of the plurality of genomic regions.

The method may also include analyzing, by the computing system, differences between the one or more additional quantitative measures and the one or more further quantitative measures to determine one or more additional variables for the predictive model.

In addition, the method may include analyzing, by the computing system, the testing sequencing reads to determine first additional quantitative measures that correspond to the individual classification regions of the plurality of classification regions, analyzing, by the computing system, the testing sequencing reads to determine second additional quantitative measures derived from the testing sequencing reads that correspond to individual control regions of a plurality of control regions, the individual control regions of the plurality of control regions having the threshold amount of methylated cytosines in subjects in which cancer is detected and in further subjects in which cancer is not detected, determining, by the computing system, additional normalized quantitative measures that correspond to the subset of the plurality of classification regions, where an individual additional normalized quantitative measure is determined according to the first additional quantitative measures and the second additional quantitative measures, and generating, by the computing system, an input vector that includes the normalized quantitative measures, where the predictive model uses the input vector to determine the probability of a homologous recombination repair deficiency being present in the additional subject.

In one or more examples, a wash of the plurality of washes is performed with a solution having a concentration of sodium chloride (NaCl) and produces a nucleic acid fraction of the number of nucleic acid fractions having a range of binding energies to MBD proteins.

In at least some examples, the method may include determining that a first nucleic acid fraction is associated with a first partition of a plurality of partitions of nucleic acids, the first partition corresponding to a first range of binding energies to MBD proteins, causing a first molecular barcode to attach to nucleic acids of the first nucleic acid fraction, the first molecular barcode being associated with the first partition, determining that a second nucleic acid fraction is associated with a second partition of the plurality of partitions of nucleic acids, the second partition corresponding to a second range of binding energies to MBD proteins different from the first range of binding energies to MBD proteins, and causing a second molecular barcode to attach to nucleic acids of the second nucleic acid fraction, the second molecular barcode being associated with the second partition.

In various examples, the method may include combining at least a portion of the number of nucleic acid fractions with an amount of restriction enzyme that cleaves molecules with one or more unmethylated cytosines to produce at least a portion of the plurality of samples used to produce the training sequence representations, where the threshold amount of methylated cytosines corresponds to a minimum frequency of methylated cytosines within a region having at least the threshold cytosine-guanine content.

The method may also include combining at least a portion of the number of nucleic acid fractions with an amount of a restriction enzyme that cleaves molecules with one or more methylated cytosines to produce at least a portion of the plurality of samples used to produce the training sequence representations, where the threshold amount of unmethylated cytosines corresponds to a maximum frequency of methylated cytosines that are not cleaved within a region having at least the threshold cytosine-guanine content. The method also includes administering to the subject, a treatment suitable for treating homologous recombination repair deficiency based on the classification as having the homologous recombination repair deficiency. The method also includes administering to the subject, a treatment suitable for treating homologous recombination repair deficiency based on the classification as having the homologous recombination repair deficiency. Other technical features may be readily apparent to one skilled in the art from the following figures, descriptions, and claims.

In one aspect, a computing apparatus includes a processor. The computing apparatus also includes a memory storing instructions that, when executed by the processor, configure the apparatus to obtain training sequence data including training sequence representations derived from a plurality of samples, individual training sequence representations including a nucleotide sequence corresponding to a fragment of a nucleic acid included in a sample of a plurality of samples and individual samples of the plurality of samples corresponding to a subject classified as having a homologous recombination repair deficiency, determine a subset of the training sequence representations that correspond to nucleic acids having at least a threshold amount of methylated cytosines in one or more regions of the nucleotide sequence, analyze the subset of training sequence representations to determine quantitative measures derived from the subset of the training sequence representations, individual quantitative measures corresponding to a classification region of a plurality of classification regions of a reference genome, individual classification regions of the plurality of classification regions having the threshold amount of methylated cytosines in subjects in which cancer is detected, analyze, using one or more computational techniques, the quantitative measures of the plurality of classification regions to determine a subset of the plurality of classification regions having at least a threshold likelihood of indicating a homology directed repair deficiency, and generate a predictive model to determine a probability of a homologous recombination repair deficiency being present in one or more additional subjects, the predictive model including a plurality of variables and a plurality of weights with individual weights of the plurality of weights corresponding to individual variables of the plurality of variables, where an individual variable of the plurality of variables corresponds to an individual classification region of the subset of the plurality of classification regions and an individual weight that corresponds to the individual variable indicates a likelihood of the individual classification region indicating a homologous recombination repair deficiency.

The computing apparatus may also include additional instructions that, when executed by the processor, configure the apparatus to analyze the subset of training sequence representations to determine additional quantitative measures derived from the subset of the training sequence reads, individual quantitative measures corresponding to a control region of a plurality of control regions of a reference genome, individual control regions of the plurality of control regions having the threshold amount of methylated cytosines in subjects in which cancer is detected and in further subjects in which cancer is not detected, and determine normalized quantitative measures that correspond to the subset of the plurality of classification regions, where an individual normalized quantitative measure is determined according to the quantitative measure that corresponds to a classification region of the subset of the plurality of classification regions and the additional quantitative measures.

In addition, the computing apparatus may include additional instructions that, when executed by the processor, configure the apparatus to determine, by implementing the predictive model, individual probabilities of a homologous recombination repair deficiency being present in individual samples of the plurality of samples based on the normalized quantitative measures corresponding to the individual samples, and determine, by the computing system and based on the individual probabilities, a threshold probability to indicate a homologous recombination repair deficiency being present with respect to a given subject.

Further, the computing apparatus may include additional instructions that, when executed by the processor, configure the apparatus to determine a responsiveness to treatment with respect to a group of subjects, where cancer is detected in the group of subjects and the treatment is provided to treat the cancer, and determine the plurality of samples that correspond to subjects having a homologous recombination repair deficiency based on the responsiveness of a portion of the group of subjects to the treatment being at least a threshold level of responsiveness.

In one or more examples, the computing apparatus may include additional instructions that, when executed by the processor, configure the apparatus to analyze additional sequence reads derived from samples of a group of subjects in which cancer is detected to determine whether one or more genomic mutations are present with respect to one or more genomic regions, where the one or more genomic mutations correspond to homologous recombination repair pathways, and determine the plurality of samples used to produce the training sequence representations by identifying a portion of the samples derived from the group of subjects in which the one or more genomic mutations are present.

In various examples, the one or more computational techniques include implementing one or more logistic regression models with elastic regularization.

In at least some examples, the computing apparatus may include additional instructions that, when executed by the processor, configure the apparatus to implement the predictive model to determine a probability of a homologous recombination repair deficiency being present in a plurality of additional samples, the plurality of additional samples being derived from additional subjects with a first form of cancer being detected in a first portion of the additional subjects and a second form of cancer being detected in a second portion of the additional subjects.

In one or more additional examples, the computing apparatus may include additional instructions that, when executed by the processor, configure the apparatus to implement the predictive model to determine a probability of a homologous recombination repair deficiency being present in a plurality of additional samples, the plurality of additional samples being derived from additional subjects in which a single form of cancer is present.

In one or more further examples, the computing apparatus may include additional instructions that, when executed by the processor, configure the apparatus to analyze the subset of training sequence reads to determine a group of training sequence reads that correspond to a plurality of genomic regions associated with homologous recombination repair pathways, and determine one or more additional quantitative measures based on a number of the group of training sequence representations that correspond to at least a portion of the plurality of genomic regions,

In various examples, the plurality of classification regions have at least a threshold amount of cytosine-guanine content.

The computing apparatus may also include additional instructions that, when executed by the processor, configure the apparatus to determine tumor fraction estimates for a number of samples, the number of samples corresponding to subjects in which cancer is detected, analyze the tumor fraction estimates with respect to a threshold tumor fraction estimate, and determine, by the computing system, the plurality of samples used to derive the training sequence reads based on identifying at least a portion of the number of samples having a tumor fraction estimate corresponding to at least the threshold tumor fraction estimate.

Additionally, the computing apparatus may include additional instructions that, when executed by the processor, configure the apparatus to obtain testing sequence data from an additional subject that is not included in the plurality of subjects, the testing sequence data including testing sequencing representations derived from a sample of the additional subject, individual testing sequencing representations including a nucleotide sequence corresponding to a fragment of a nucleic acid included in the additional sample and individual testing sequencing reads corresponding to molecules having the threshold amount of methylated cytosines included in regions of the nucleotide, and determine, using the predictive model and the additional sequence data, a probability of a homologous recombination repair deficiency being present in the additional subject.

The computing apparatus may also include additional instructions that, when executed by the processor, configure the apparatus to determine, for individual classification regions of the plurality of classification regions, differences between a first portion of the normalized quantitative measures derived from samples that correspond to subjects in which a homology directed repair deficiency is present and a second portion of the normalized quantitative measures derived from samples that correspond to additional subjects in which a homologous recombination repair deficiency is not present, and determine that an individual classification region is included in the subset of the plurality of classification regions based on the difference between the first portion of the normalized quantitative measures for the individual classification region and the second portion of the normalized quantitative measures for the individual classification region being at least a threshold difference.

In one or more examples, the treatment is a poly adenosine diphosphate (ADP) ribose polymerase (PARP) inhibitor.

In addition, the computing apparatus may include additional instructions that, when executed by the processor, configure the apparatus to determine an additional subset of the training sequence representations that correspond to additional nucleic acids having less than an additional threshold amount of methylation, analyze the additional subset of the training sequence reads to determine an additional group of training sequence representations that correspond to the plurality of genomic regions associated with the homologous recombination repair pathways, determine one or more further quantitative measures based on an additional number of the additional group of training sequence representations that correspond to at least a portion of the plurality of genomic regions.

Further, the computing apparatus may include additional instructions that, when executed by the processor, configure the apparatus to analyze differences between the one or more additional quantitative measures and the one or more further quantitative measures to determine one or more additional variables for the predictive model.

In various examples, the computing apparatus may include additional instructions that, when executed by the processor, configure the apparatus to analyze the testing sequencing reads to determine first additional quantitative measures that correspond to the individual classification regions of the plurality of classification regions, analyze the testing sequencing reads to determine second additional quantitative measures derived from the testing sequencing reads that correspond to individual control regions of a plurality of control regions, the individual control regions of the plurality of control regions having the threshold amount of methylated cytosines in subjects in which cancer is detected and in further subjects in which cancer is not detected, determine additional normalized quantitative measures that correspond to the subset of the plurality of classification regions, where an individual additional normalized quantitative measure is determined according to the first additional quantitative measures and the second additional quantitative measures, and generate an input vector that includes the normalized quantitative measures, where the predictive model uses the input vector to determine the probability of a homologous recombination repair deficiency being present in the additional subject.

In one or more examples, the computing apparatus includes additional instructions that, when executed by the hardware processor, cause the hardware processor to determine, for individual classification regions of the plurality of classification regions, differences between a first portion of the normalized quantitative measures derived from samples that correspond to subjects in which a homology directed repair deficiency is present and a second portion of the normalized quantitative measures derived from samples that correspond to additional subjects in which a homologous recombination repair deficiency is not present, and determine that an individual classification region is included in the subset of the plurality of classification regions based on the difference between the first portion of the normalized quantitative measures for the individual classification region and the second portion of the normalized quantitative measures for the individual classification region being at least a threshold difference.

In one aspect, one or more non-transitory computer-readable storage media, include instructions that when executed by a computer, cause the computer to obtain training sequence data including training sequence representations derived from a plurality of samples, individual training sequence representations including a nucleotide sequence corresponding to a fragment of a nucleic acid included in a sample of a plurality of samples and individual samples of the plurality of samples corresponding to a subject classified as having a homologous recombination repair deficiency, determine a subset of the training sequence representations that correspond to nucleic acids having at least a threshold amount of methylated cytosines in one or more regions of the nucleotide sequence, analyze the subset of training sequence representations to determine quantitative measures derived from the subset of the training sequence representations, individual quantitative measures corresponding to a classification region of a plurality of classification regions of a reference genome, individual classification regions of the plurality of classification regions having the threshold amount of methylated cytosines in subjects in which cancer is detected, analyze, using one or more computational techniques, the quantitative measures of the plurality of classification regions to determine a subset of the plurality of classification regions having at least a threshold likelihood of indicating a homology directed repair deficiency, and generate a predictive model to determine a probability of a homologous recombination repair deficiency being present in one or more additional subjects, the predictive model including a plurality of variables and a plurality of weights with individual weights of the plurality of weights corresponding to individual variables of the plurality of variables, where an individual variable of the plurality of variables corresponds to an individual classification region of the subset of the plurality of classification regions and an individual weight that corresponds to the individual variable indicates a likelihood of the individual classification region indicating a homologous recombination repair deficiency.

The one or more non-transitory computer-readable storage media may also include instructions that when executed by a computer, cause the computer to include analyze the subset of training sequence representations to determine additional quantitative measures derived from the subset of the training sequence reads, individual quantitative measures corresponding to a control region of a plurality of control regions of a reference genome, individual control regions of the plurality of control regions having the threshold amount of methylated cytosines in subjects in which cancer is detected and in further subjects in which cancer is not detected, and determine normalized quantitative measures that correspond to the subset of the plurality of classification regions, where an individual normalized quantitative measure is determined according to the quantitative measure that corresponds to a classification region of the subset of the plurality of classification regions and the additional quantitative measures.

In addition, the one or more non-transitory computer-readable storage media may include instructions that when executed by a computer, cause the computer to determine, by implementing the predictive model, individual probabilities of a homologous recombination repair deficiency being present in individual samples of the plurality of samples based on the normalized quantitative measures corresponding to the individual samples, and determine, based on the individual probabilities, a threshold probability to indicate a homologous recombination repair deficiency being present with respect to a given subject.

Further, the one or more non-transitory computer-readable storage media may include instructions that when executed by a computer, cause the computer to determine a responsiveness to treatment with respect to a group of subjects, where cancer is detected in the group of subjects and the treatment is provided to treat the cancer, and determine the plurality of samples that correspond to subjects having a homologous recombination repair deficiency based on the responsiveness of a portion of the group of subjects to the treatment being at least a threshold level of responsiveness.

In one or more examples, the one or more non-transitory computer-readable storage media may include instructions that when executed by a computer, cause the computer to analyze additional sequence reads derived from samples of a group of subjects in which cancer is detected to determine whether one or more genomic mutations are present with respect to one or more genomic regions, where the one or more genomic mutations correspond to homologous recombination repair pathways, and determine the plurality of samples used to produce the training sequence representations by identifying a portion of the samples derived from the group of subjects in which the one or more genomic mutations are present.

The one or more computational techniques include implementing one or more logistic regression models with elastic regularization.

In one or more additional examples, the one or more non-transitory computer-readable storage media may include instructions that when executed by a computer, cause the computer to implement the predictive model to determine a probability of a homologous recombination repair deficiency being present in a plurality of additional samples, the plurality of additional samples being derived from additional subjects with a first form of cancer being detected in a first portion of the additional subjects and a second form of cancer being detected in a second portion of the additional subjects.

In one or more further examples, the one or more non-transitory computer-readable storage media may include instructions that when executed by a computer, cause the computer to implement the predictive model to determine a probability of a homologous recombination repair deficiency being present in a plurality of additional samples, the plurality of additional samples being derived from additional subjects in which a single form of cancer is present.

In at least some examples the one or more non-transitory computer-readable storage media may include instructions that when executed by a computer, cause the computer to analyze the subset of training sequence reads to determine a group of training sequence reads that correspond to a plurality of genomic regions associated with homologous recombination repair pathways, and determine one or more additional quantitative measures based on a number of the group of training sequence representations that correspond to at least a portion of the plurality of genomic regions,

In various examples, the plurality of classification regions have at least a threshold amount of cytosine-guanine content.

The one or more non-transitory computer-readable storage media may also include instructions that when executed by a computer, cause the computer to determine tumor fraction estimates for a number of samples, the number of samples corresponding to subjects in which cancer is detected, analyze the tumor fraction estimates with respect to a threshold tumor fraction estimate, and determine the plurality of samples used to derive the training sequence reads based on identifying at least a portion of the number of samples having a tumor fraction estimate corresponding to at least the threshold tumor fraction estimate.

Additionally, the one or more non-transitory computer-readable storage media may include instructions that when executed by a computer, cause the computer to obtain testing sequence data from an additional subject that is not included in the plurality of subjects, the testing sequence data including testing sequencing representations derived from a sample of the additional subject, individual testing sequencing representations including a nucleotide sequence corresponding to a fragment of a nucleic acid included in the additional sample and individual testing sequencing reads corresponding to molecules having the threshold amount of methylated cytosines included in regions of the nucleotide, and determine, using the predictive model and the additional sequence data, a probability of a homologous recombination repair deficiency being present in the additional subject.

The treatment may include a poly adenosine diphosphate (ADP) ribose polymerase (PARP) inhibitor.

The one or more non-transitory computer-readable storage media may also include instructions that when executed by a computer, cause the computer to determine an additional subset of the training sequence representations that correspond to additional nucleic acids having less than an additional threshold amount of methylation, analyze, by the computing system, the additional subset of the training sequence reads to determine an additional group of training sequence representations that correspond to the plurality of genomic regions associated with the homologous recombination repair pathways, determine one or more further quantitative measures based on an additional number of the additional group of training sequence representations that correspond to at least a portion of the plurality of genomic regions.

Additionally, the one or more non-transitory computer-readable storage media may include instructions that when executed by a computer, cause the computer to analyze differences between the one or more additional quantitative measures and the one or more further quantitative measures to determine one or more additional variables for the predictive model.

Further, the one or more non-transitory computer-readable storage media may include instructions that when executed by a computer, cause the computer to analyze the testing sequencing reads to determine first additional quantitative measures that correspond to the individual classification regions of the plurality of classification regions, analyze, by the computing system, the testing sequencing reads to determine second additional quantitative measures derived from the testing sequencing reads that correspond to individual control regions of a plurality of control regions, the individual control regions of the plurality of control regions having the threshold amount of methylated cytosines in subjects in which cancer is detected and in further subjects in which cancer is not detected, determine additional normalized quantitative measures that correspond to the subset of the plurality of classification regions, where an individual additional normalized quantitative measure is determined according to the first additional quantitative measures and the second additional quantitative measures, and generate an input vector that includes the normalized quantitative measures, where the predictive model uses the input vector to determine the probability of a homologous recombination repair deficiency being present in the additional subject.

### Definitions

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons of ordinary skill in the art upon reading this disclosure and so forth.

It is also to be understood that the terminology used herein is for the purpose of describing particular implementations only, and is not intended to be limiting. Further, unless defined otherwise, all technical and scientific terms used herein have the same meaning as commonly understood by one of ordinary skill in the art to which this disclosure pertains. In describing and claiming the methods, computer readable media, and systems, the following terminology, and grammatical variants thereof, will be used in accordance with the definitions set forth below.

About. As used herein, “about” or “approximately” as applied to one or more values or elements of interest, refers to a value or element that is similar to a stated reference value or element. In certain implementations, the term “about” or “approximately” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Administer: As used herein, “administer” or “administering” a therapeutic agent (e.g., an immunological therapeutic agent) to a subject means to give, apply or bring the composition into contact with the subject. Administration can be accomplished by any of a number of routes, including, for example, topical, oral, subcutaneous, intramuscular, intraperitoneal, intravenous, intrathecal and intradermal.

Adapter. As used herein, “adapter” refers to a short nucleic acid (e.g., less than about 500 nucleotides, less than about 100 nucleotides, or less than about 50 nucleotides in length) that can be at least partially double-stranded and used to link to either or both ends of a given sample nucleic acid molecule. Adapters can include nucleic acid primer binding sites to permit amplification of a nucleic acid molecule flanked by adapters at both ends, and/or a sequencing primer binding site, including primer binding sites for sequencing applications, such as various next-generation sequencing (NGS) applications. Adapters can also include binding sites for capture probes, such as an oligonucleotide attached to a flow cell support or the like. Adapters can also include a nucleic acid tag as described herein. Nucleic acid tags can be positioned relative to amplification primer and sequencing primer binding sites, such that a nucleic acid tag is included in amplicons and sequence reads of a given nucleic acid molecule. The same or different adapters can be linked to the respective ends of a nucleic acid molecule. In some implementations, the same adapter is linked to the respective ends of the nucleic acid molecule except that the nucleic acid tag differs. In some implementations, the adapter is a Y-shaped adapter in which one end is blunt ended or tailed as described herein, for joining to a nucleic acid molecule, which is also blunt ended or tailed with one or more complementary nucleotides. In still other example implementations, an adapter is a bell-shaped adapter that includes a blunt or tailed end for joining to a nucleic acid molecule to be analyzed. Other examples of adapters include T-tailed and C-tailed adapters.

Alignment. As used herein, “alignment” or “align” refers to determining whether at least two sequence representations have at least a threshold amount of homology. In one or more examples, the threshold amount of homology can be at least about 90%, at least about 91%, at least about 92%, at least about 93%, at least about 94%, at least about 95%, at least about 96%, at least about 97%, at least about 98%, at least about 99%, at least about 99.5%, or at least about 99.9%. In situations where two sequence representations have at least the threshold amount of homology, the two sequence representations can be referred to as being “aligned.”

Amplify. As used herein, “amplify” or “amplification” in the context of nucleic acids refers to the production of multiple copies of a polynucleotide, or a portion of the polynucleotide, starting from a small amount of the polynucleotide (e.g., a single polynucleotide molecule), where the amplification products or amplicons are generally detectable. Amplification of polynucleotides encompasses a variety of chemical and enzymatic processes.

Barcode: As used herein, “barcode” or “molecular barcode” in the context of nucleic acids refers to a nucleic acid molecule comprising a sequence that can serve as a molecular identifier. For example, individual “barcode” sequences can be added to each DNA fragment during next-generation sequencing (NGS) library preparation so that each read can be identified and sorted before the final data analysis.

Cancer Type: As used herein, “cancer type” refers to a type or subtype of cancer defined, e.g., by histopathology. Cancer type can be defined by any conventional criterion, such as on the basis of occurrence in a given tissue (e.g., blood cancers, central nervous system (CNS), brain cancers, lung cancers (small cell and non-small cell), skin cancers, nose cancers, throat cancers, liver cancers, bone cancers, lymphomas, pancreatic cancers, bowel cancers, rectal cancers, thyroid cancers, bladder cancers, kidney cancers, mouth cancers, stomach cancers, breast cancers, prostate cancers, ovarian cancers, lung cancers, intestinal cancers, soft tissue cancers, neuroendocrine cancers, gastroesophageal cancers, head and neck cancers, gynecological cancers, colorectal cancers, urothelial cancers, solid state cancers, heterogeneous cancers, homogenous cancers), unknown primary origin and the like, and/or of the same cell lineage (e.g., carcinoma, sarcoma, lymphoma, cholangiocarcinoma, leukemia, mesothelioma, melanoma, or glioblastoma) and/or cancers exhibiting cancer markers, such as Her2, CA15-3, CA19-9, CA-125, CEA, AFP, PSA, HCG, hormone receptor and NMP-22. Cancers can also be classified by stage (e.g., stage 1, 2, 3, or 4) and whether of primary or secondary origin.

Carrier Signal: As used herein, “carrier signal” refers to any intangible medium that is capable of storing, encoding, or carrying transitory or non-transitory instructions 502 for execution by the machine 500, and includes digital or analog communications signals or other intangible medium to facilitate communication of such instructions 502. Instructions 502 may be transmitted or received over the network 534 using a transitory or non-transitory transmission medium via a network interface device and using any one of a number of well-known transfer protocols.

Cell-Free Nucleic Acid: As used herein, “cell-free nucleic acid” refers to nucleic acids not contained within or otherwise bound to a cell or, in some implementations, nucleic acids remaining in a sample following the removal of intact cells. Cell-free nucleic acids can include, for example, all non-encapsulated nucleic acids sourced from a bodily fluid (e.g., blood, plasma, serum, urine, cerebrospinal fluid (CSF), etc.) from a subject. Cell-free nucleic acids include DNA (cfDNA), RNA (cfRNA), and hybrids thereof, including genomic DNA, mitochondrial DNA, circulating DNA, siRNA, miRNA, circulating RNA (cRNA), tRNA, rRNA, small nucleolar RNA (snoRNA), Piwi-interacting RNA (piRNA), long non-coding RNA (long ncRNA), and/or fragments of any of these. Cell-free nucleic acids can be double-stranded, single-stranded, or a hybrid thereof. A cell-free nucleic acid can be released into bodily fluid through secretion or cell death processes, e.g., cellular necrosis, apoptosis, or the like. Some cell-free nucleic acids are released into bodily fluid from cancer cells, e.g., circulating tumor DNA (ctDNA). Others are released from healthy cells. CtDNA can be non-encapsulated tumor-derived fragmented DNA. A cell-free nucleic acid can have one or more epigenetic modifications, for example, a cell-free nucleic acid can be acetylated, 5-methylated, ubiquitylated, phosphorylated, sumoylated, ribosylated, and/or citrullinated.

Cellular Nucleic Acids: As used herein, “cellular nucleic acids” means nucleic acids that are disposed within one or more cells at least at the point a sample is taken or collected from a subject, even if those nucleic acids are subsequently removed as part of a given analytical process.

Classification Region: As used herein, “classification region” refers to a genomic region that may show sequence-independent changes in neoplastic cells (e.g., tumor cells and cancer cells) or that may show sequence-independent changes in cfDNA from subjects having cancer relative to cfDNA from subjects in which cancer is not present. “Classification region” can also refer to a genomic region that is associated with a homologous recombination pathway. Examples of sequence-independent changes include, but are not limited to, changes in methylation rate (increases or decreases), nucleosome distribution, CTCF binding, transcription start sites, and regulatory protein binding regions The classification region can be enriched by one or more probes. In addition, the classification region can be defined by a pair of primer binding sites. Further, the classification region can be defined by a predetermined beginning genomic locus and a predetermined ending genomic locus. The classification region can include from about 25 nucleotides to about 250 nucleotides, from about 50 nucleotides to about 200 nucleotides, or from about 75 nucleotides to about 150 nucleotides.

Communications Network. As used herein, “communications network” refers to one or more portions of a network 114, 1034 that may be an ad hoc network, an intranet, an extranet, a virtual private network (VPN), a local area network (LAN), a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network 114, 1034 or a portion of a network may include a wireless or cellular network and the coupling may be a Code Division Multiple Access (CDMA) connection, a Global System for Mobile communications (GSM) connection, or other type of cellular or wireless coupling. In this example, the coupling may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

Coverage: As used herein, “coverage” or “coverage metrics” refer to the number of nucleic acid molecules or sequencing reads that correspond to a particular genomic region of a reference sequence.

Deoxyribonucleic Acid or Ribonucleic Acid: As used herein, “deoxyribonucleic acid” or “DNA” refers to a natural or modified nucleotide which has a hydrogen group at the 2′-position of the sugar moiety. DNA can include a chain of nucleotides comprising four types of nucleotide bases: adenine (A), thymine (T), cytosine (C), and guanine (G). As used herein, “ribonucleic acid” or “RNA” refers to a natural or modified nucleotide which has a hydroxyl group at the 2′-position of the sugar moiety. RNA can include a chain of nucleotides comprising four types of nucleotides: A, uracil (U), G, and C. As used herein, the term “nucleotide” refers to a natural nucleotide or a modified nucleotide. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data”, “nucleic acid sequencing information”, “sequence information”, “sequence representation”, “nucleic acid sequence”, “nucleotide sequence”, “genomic sequence”, “genetic sequence”, “fragment sequence”, “sequencing read”, or “nucleic acid sequencing read” denotes any information or data that is indicative of the order and identity of the nucleotide bases (e.g., adenine, guanine, cytosine, and thymine or uracil) in a molecule (e.g., a whole genome, whole transcriptome, exome, oligonucleotide, polynucleotide, or fragment) of a nucleic acid such as DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, and electronic signature-based systems.

Driver Mutation: As used herein, “driver mutation” means a mutation that drives cancer progression.”

Homologous Recombination Deficiency. As uses herein “homologous recombination deficiency” or HRD or “homologous recombination repair deficiency” refers to the inability of cells to effectively repair double stranded DNA breaks using homologous recombination repair pathways. HRD is typically characterized by mutations of one or more genomic regions that regulate homologous recombination repair pathways.

Homologous Recombination Repair Pathway: As uses herein “homologous recombination repair pathway” refers to one or more processes that use a group of proteins to repair damage to DNA caused by double stranded breaks.

Hypermethylation: As used herein, “hypermethylation” refers to an increased level or degree of methylation of nucleic acid molecule(s) relative to the other nucleic acid molecules within a population (e.g., sample) of nucleic acid molecules from the same genomic locus. In some embodiments, hypermethylated DNA can include DNA molecules comprising at least 1 methylated cytosine, at least 2 methylated cytosines, at least 3 methylated cytosines, at least 5 methylated cytosines, or at least 10 methylated cytosines.

Hypomethylation: As used herein, “hypomethylation” refers to a decreased level or degree of methylation of nucleic acid molecule(s) relative to the other nucleic acid molecules within a population (e.g., sample) of nucleic acid molecules from the same genomic locus. In some embodiments, hypomethylated DNA includes unmethylated DNA molecules. In some embodiments, hypomethylated DNA can include DNA molecules comprising 0 methylated cytosine, at most 1 methylated cytosine, at most 2 methylated cytosines, at most 3 methylated cytosines, at most 4 methylated cytosines, or at most 5 methylated cytosines.

Immunotherapy: As used herein, “immunotherapy” refers to treatment with one or more agents that act to stimulate the immune system so as to kill or at least to inhibit growth of cancer cells, and preferably to reduce further growth of the cancer, reduce the size of the cancer and/or eliminate the cancer. Some such agents bind to a target present on cancer cells; some bind to a target present on immune cells and not on cancer cells; some bind to a target present on both cancer cells and immune cells. Such agents include, but are not limited to, checkpoint inhibitors and/or antibodies. Checkpoint inhibitors are inhibitors of pathways of the immune system that maintain self-tolerance and modulate the duration and amplitude of physiological immune responses in peripheral tissues to minimize collateral tissue damage (see, e.g., Pardoll, Nature Reviews Cancer 12, 252-264 (2012)). Example agents include antibodies against any of PD-1, PD-2, PD-L1, PD-L2, CTLA-40, OX40, B7.1, B7He, LAG3, CD137, KIR, CCR5, CD27, or CD40. Other example agents include proinflammatory cytokines, such as IL-1β, IL-6, and TNF-α. Other example agents are T-cells activated against a tumor, such as T-cells activated by expressing a chimeric antigen targeting a tumor antigen recognized by the T-cell.

Indel: As used herein, “indel” refers to a mutation that involves the insertion or deletion of nucleotides in the genome of a subject.

Machine-Readable Medium: As used herein, “machine-readable medium” refers to a component, device, or other tangible media able to store instructions 502 and data temporarily or permanently and may include, but is not limited to, random-access memory (RAM), read-only memory (ROM), buffer memory, flash memory, optical media, magnetic media, cache memory, other types of storage (e.g., erasable programmable read-only memory (EEPROM)) and/or any suitable combination thereof. The term “machine-readable medium” may be taken to include a single medium or multiple media (e.g., a centralized or distributed database, or associated caches and servers) able to store instructions 502. The term “machine-readable medium” shall also be taken to include any medium, or combination of multiple media, that is capable of storing instructions 502 (e.g., code) for execution by a machine 500, such that the instructions 502, when executed by one or more processors 504 of the machine 500, cause the machine 500 to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” excludes signals per se.

Maximum MAF: As used herein, “maximum MAF” or “max MAF” refers to the maximum MAF of all somatic variants in a sample.

Methylation: As used herein, “methylation” or “DNA methylation” refers to addition of a methyl group to a nucleotide base in a nucleic acid molecule. In some embodiments, methylation refers to addition of a methyl group to a cytosine at a CpG site (cytosine-phosphate-guanine site (i.e., a cytosine followed by a guanine in a 5′→3′ direction of the nucleic acid sequence). In some embodiments, DNA methylation refers to addition of a methyl group to adenine, such as in N⁶-methyladenine. In some embodiments, DNA methylation is 5-methylation (modification of the 5th carbon of the 6-carbon ring of cytosine). In some embodiments, 5-methylation refers to addition of a methyl group to the 5C position of the cytosine to create 5-methylcytosine (5mC). In some embodiments, methylation comprises a derivative of 5mC. Derivatives of 5mC include, but are not limited to, 5-hydroxymethylcytosine (5-hmC), 5-formylcytosine (5-fC), and 5-caryboxylcytosine (5-caC). In some embodiments, DNA methylation is 3C methylation (modification of the 3rd carbon of the 6-carbon ring of cytosine). In some embodiments, 3C methylation comprises addition of a methyl group to the 3C position of the cytosine to generate 3-methylcytosine (3mC). Methylation can also occur at non CpG sites, for example, methylation can occur at a CpA, CpT, or CpC site. DNA methylation can change the activity of methylated DNA region. For example, when DNA in a promoter region is methylated, transcription of the gene may be repressed. DNA methylation is critical for normal development and abnormality in methylation may disrupt epigenetic regulation. The disruption, e.g., repression, in epigenetic regulation may cause diseases, such as cancer. Promoter methylation in DNA may be indicative of cancer.

Methylation-Dependent Nuclease: As used herein, “methylation-dependent nuclease” refers to a nuclease that preferentially cuts methylated DNA relative to unmethylated DNA. For example, a methylation-dependent nuclease may cut at or near a recognition sequence such as a restriction site in a manner dependent on methylation of at least one of the nucleobases in the recognition sequence, such as a cytosine. In some embodiments, the nucleolytic activity of the methylation-dependent nuclease is at least 10, 20, 50, or 100-fold higher on a methylated recognition site relative to an unmethylated control in a standard nucleolysis assay. Methylation-dependent nucleases include methylation-dependent restriction enzymes.

Methylation-Dependent Restriction Enzyme: As used herein, “methylation-dependent restriction enzyme” or “MDRE” refers to a restriction enzyme that is dependent on methylation of the DNA (e.g., cytosine methylation) i.e., the presence or absence of methyl group in a nucleotide base alters the rate at which the enzyme cleaves the target DNA. In some embodiments, the methylation dependent restriction enzymes do not cleave the DNA if a particular nucleotide base is unmethylated at the recognition sequence. For example, MspJI is a methylation dependent restriction enzyme with a recognition sequence “mCNNR (N9)” and it does not cleave DNA if the absence of the methylated cytosine (mC) in the recognition sequence.

Methylation-Sensitive Nuclease: As used herein, “methylation-sensitive nuclease” refers to a nuclease that preferentially cuts unmethylated DNA relative to methylated DNA. For example, a methylation-sensitive nuclease may cut at or near a recognition sequence such as a restriction site in a manner dependent on lack of methylation of at least one of the nucleobases in the recognition sequence, such as a cytosine. In some embodiments, the nucleolytic activity of the methylation-sensitive nuclease is at least 10, 20, 50, or 100-fold higher on an unmethylated recognition site relative to a methylated control in a standard nucleolysis assay. Methylation-sensitive nucleases include methylation-sensitive restriction enzymes.

Methylation Sensitive Restriction Enzyme: As used herein, “methylation sensitive restriction enzyme” or “MSRE” refers to a restriction enzyme that is sensitive to the methylation status of the DNA (e.g., cytosine methylation) i.e., the presence or absence of methyl group in a nucleotide base alters the rate at which the enzyme cleaves the target DNA. In some embodiments, the methylation sensitive restriction enzymes do not cleave the DNA if a particular nucleotide base is methylated at the recognition sequence. For example, HpaII is a methylation sensitive restriction enzyme with a recognition sequence “CCGG” and it does not cleave DNA if the second cytosine in the recognition sequence is methylated.

Methylation rate: As used herein, “methylation rate” refers to the probability, likelihood, or percentage that a given base (for example: cytosine residue in a CpG) is methylated on a DNA molecule at a particular genomic region analyzed in the sample. In some embodiments, the methylation rate may be applied to a defined region that comprises one or more potentially methylated bases. In some embodiments, the methylation rate refers to the percentage of CpG residues methylated in a DNA molecule. In some embodiments, the methylation rate refers to the percentage of CpG residues methylated in molecules aligned to particular genomic position or genomic region. Methylation rate can be measured by a variety of methods including, but not limited to, either using bisulfite sequencing (any single base resolution like TAPS, EM-SEQ, etc.) or using partitioning (DNA molecule resolution). Methylation rate can be measured in different ways. One estimation can be by counting how many DNA fragments end up in each methylation dependent partition or by counting the number of converted CpGs per fragment in the case of bisulfite sequencing. In addition, in the case of methylation dependent partitioning, the rate calculation can be normalized using a set of predefined regions with known methylation state or spiked-in synthetic DNA with known methylation state, deriving rate-parametrized partition distributions and estimating the rate using a maximum likelihood approach.

Methylation Status: As used herein, “methylation status” can refer to the presence or absence of methyl group on a DNA base (e.g., cytosine) at a particular genomic position in a nucleic acid molecule. It can also refer to the degree of methylation in a nucleic acid sequence (e.g., highly methylated, low methylated, intermediately methylated or unmethylated nucleic acid molecules). The methylation status can also refer to the number of nucleotides methylated in a particular nucleic acid molecule.

Modified Nucleotide Specific Binding Reagent: As used herein, refers to a binding reagent that is specific for, or targets, modified nucleotides. For example, a modified nucleotide can be a nucleotide that has been methylated, thus, the binding reagent can be specific for a methylated nucleotide. Examples of binding reagents include, but are not limited to, a methyl binding domain (MBD) of a methylation binding protein (“MBP”) or variants thereof, an antibody (and antibody variants e.g., single chain antibodies), aptamers, or combinations thereof. Thus, as disclosed throughout, the use of MBD can be exchanged for any other modified nucleotide specific binding reagent, provided the modified nucleotide specific binding reagent has the desired specificity and affinity for the specific modified base of interest in the selected implementation.

Mutant Allele Fraction: As used herein, “mutant allele fraction”, “mutation dose,” or “MAF” refers to the fraction of nucleic acid molecules harboring an allelic alteration or mutation at a given genomic position in a given sample. MAF is generally expressed as a fraction or a percentage. For example, an MAF can be less than about 0.5, 0.1, 0.05, or 0.01 (i.e., less than about 50%, 10%, 5%, or 1%) of all somatic variants or alleles present at a given locus.

Mutation: As used herein, “mutation” refers to a variation from a known reference sequence and includes mutations such as, for example, single nucleotide variants (SNVs), copy number variants or variations (CNVs)/aberrations, insertions or deletions (indels), gene fusions, transversions, translocations, frame shifts, duplications, repeat expansions, and epigenetic variants. A mutation can be a germline or somatic mutation. In some examples, a reference sequence for purposes of comparison is a wildtype genomic sequence of the species of the subject providing a test sample, typically the human genome.

Mutation Caller. As used herein, “mutation caller” means an algorithm (embodied in software or otherwise computer implemented) that is used to identify mutations in test sample data (e.g., sequence information obtained from a subject).

Mutation Count: As used herein, “mutation count” or “mutational count” refers to the number of somatic mutations in a whole genome or exome or targeted regions of a nucleic acid sample.

Negative Control Region: As used herein, “negative control region”, refers to a genomic region that includes less than a threshold number of nucleic acids with cytosines that are methylated in cells that are derived from subjects that are free of cancer and also subjects in which cancer is not present.

Neoplasm: As used herein, the terms “neoplasm” and “tumor” are used interchangeably. They refer to abnormal growth of cells in a subject. A neoplasm or tumor can be benign, potentially malignant, or malignant. A malignant tumor is referred to as a cancer or a cancerous tumor.

Next Generation Sequencing: As used herein, “next generation sequencing” or “NGS” refers to sequencing technologies having increased throughput as compared to traditional Sanger- and capillary electrophoresis-based approaches, for example, with the ability to generate hundreds of thousands of relatively small sequencing reads at a time. Some examples of next generation sequencing techniques include, but are not limited to, sequencing by synthesis, sequencing by ligation, and sequencing by hybridization.

Nucleic Acid Tag: As used herein, “nucleic acid tag” refers to a short nucleic acid (e.g., less than about 500 nucleotides, about 100 nucleotides, about 50 nucleotides, or about 10 nucleotides in length), used to distinguish nucleic acids from different samples (e.g., representing a sample index), or different nucleic acid molecules in the same sample (e.g., representing a molecular barcode), of different types, or which have undergone different processing. The nucleic acid tag comprises a predetermined, fixed, non-random, random or semi-random oligonucleotide sequence. Such nucleic acid tags may be used to label different nucleic acid molecules or different nucleic acid samples or sub-samples. Nucleic acid tags can be single-stranded, double-stranded, or at least partially double-stranded. Nucleic acid tags optionally have the same length or varied lengths. Nucleic acid tags can also include double-stranded molecules having one or more blunt-ends, include 5′ or 3′ single-stranded regions (e.g., an overhang), and/or include one or more other single-stranded regions at other locations within a given molecule. Nucleic acid tags can be attached to one end or to both ends of the other nucleic acids (e.g., sample nucleic acids to be amplified and/or sequenced). Nucleic acid tags can be decoded to reveal information such as the sample of origin, form, or processing of a given nucleic acid. For example, nucleic acid tags can also be used to enable pooling and/or parallel processing of multiple samples comprising nucleic acids bearing different molecular barcodes and/or sample indexes in which the nucleic acids are subsequently being deconvolved by detecting (e.g., reading) the nucleic acid tags. Nucleic acid tags can also be referred to as identifiers (e.g., molecular identifier, sample identifier). Additionally, or alternatively, nucleic acid tags can be used as molecular identifiers (e.g., to distinguish between different molecules or amplicons of different parent molecules in the same sample or sub-sample). This includes, for example, uniquely tagging different nucleic acid molecules in a given sample, or non-uniquely tagging such molecules. In the case of non-unique tagging applications, a limited number of tags (i.e., molecular barcodes) may be used to tag each nucleic acid molecule such that different molecules can be distinguished based on their endogenous sequence information (for example, start and/or stop positions where they map to a selected reference sequence, a sub-sequence of one or both ends of a sequence, and/or length of a sequence) in combination with at least one molecular barcode. A sufficient number of different molecular barcodes are used such that there is a low probability (e.g., less than about a 10%, less than about a 5%, less than about a 1%, or less than about a 0.1% chance) that any two molecules may have the same endogenous sequence information (e.g., start and/or stop positions, subsequences of one or both ends of a sequence, and/or lengths) and also have the same molecular barcode.

Partitioning: As used herein, “partitioning” refers to physically separating or fractionating a mixture of nucleic acid molecules in a sample based on a characteristic of the nucleic acid molecules. The partitioning can be physical partitioning of molecules. Partitioning can involve separating the nucleic acid molecules into groups or sets based on the level of epigenetic feature (for e.g., methylation). For example, the nucleic acid molecules can be partitioned based on the level of methylation of the nucleic acid molecules. In some embodiments, the methods and systems used for partitioning may be found in PCT Patent Application No. PCT/US2017/068329, which is hereby incorporated by reference in its entirety.

Partitioned set: As used herein, “partitioned set” or “partition” refers to a set of nucleic acid molecules partitioned into a set or group based on the differential binding affinity of the nucleic acid molecules or proteins associated with the nucleic acid molecules to a binding agent. A partitioned set may also be referred to as a subsample. The binding agent binds preferentially to the nucleic acid molecules comprising nucleotides with epigenetic modification. For example, if the epigenetic modification is methylation, the binding agent can be a methyl binding domain (MBD) protein. In some embodiments, a partitioned set can comprise nucleic acid molecules belonging to a particular level or degree of epigenetic feature (for e.g., methylation). For example, the nucleic acid molecules can be partitioned into three sets-one set for highly methylated nucleic acid molecules (first subsample, hyper partition, hyper partitioned set or hypermethylated partitioned set), a second set for low methylated nucleic acid molecules (second subsample, hypo partition, hypo partitioned set or hypomethylated partitioned set), and a third set for intermediate methylated nucleic acid molecules (third subsample, intermediate partitioned set, intermediately methylated partitioned set, residual partition, or residual partitioned set). In another example, the nucleic acid molecules can be partitioned based on the number of methylated nucleotides-one partitioned set can have nucleic acid molecules with nine methylated nucleotides, and another partitioned set can have unmethylated nucleic acid molecules (zero methylated nucleotides).

Polynucleotide: As used herein, “polynucleotide”, “nucleic acid”, “nucleic acid molecule”, “polynucleotide molecule”, or “oligonucleotide” refers to a linear polymer of nucleosides (including deoxyribonucleosides, ribonucleosides, or analogs thereof) joined by internucleosidic linkages. A polynucleotide can comprise at least three nucleosides. Oligonucleotides often range in size from a few monomeric units, e.g., 3-4, to hundreds of monomeric units. Whenever a polynucleotide is represented by a sequence of letters, such as “ATGCCTG,” it will be understood that the nucleotides are in 5′→3′ order from left to right and that in the case of DNA, “A” denotes deoxyadenosine, “C” denotes deoxycytidine, “G” denotes deoxyguanosine, and “T” denotes deoxythymidine, unless otherwise noted. The letters A, C, G, and T may be used to refer to the bases themselves, to nucleosides, or to nucleotides comprising the bases, as is standard in the art.

Positive Control Region: As used herein, “positive control region”, refers to a genomic region that includes at least a threshold number of nucleic acids with cytosines that are methylated in cells and that are derived from both subjects that are free of homologous recombination repair deficiencies and from subjects in which recombination repair deficiencies are present.

Probe: As used herein, “probe” refers to a polynucleotide comprising a functionality. The functionality can be a detectable label (fluorescent), a binding moiety (biotin), or a solid support (a magnetically attractable particle or a chip). Probes can include single-stranded DNA/RNA polynucleotides or double stranded DNA polynucleotides that hybridize to target nucleic acid sequences (e.g., SureSelect® probes, Agilent Technologies). Sequence capture using probes generally depends, in part, on the number of consecutive nucleotides in at least a portion of the target nucleic acid sequence that is complementary (or nearly complementary) to the sequence of the probe. In some examples, probes can correspond to driver mutations.

Processing: As used herein, the terms “processing”, “calculating”, and “comparing” can be used interchangeably. In certain applications, the terms refer to determining a difference, e.g., a difference in number or sequence. For example, gene expression, copy number variation (CNV), indel, and/or single nucleotide variant (SNV) values or sequences can be processed.

Processor. As used herein, “processor” refers to any circuit or virtual circuit (a physical circuit emulated by logic executing on an actual processor) that manipulates data values according to control signals (e.g., “commands,” “op codes,” “machine code,” etc.) and which produces corresponding output signals that are applied to operate a machine. A processor may, for example, be a CPU, a RISC processor, a CISC processor, a GPU, a DSP, an ASIC, a RFIC or any combination thereof. A processor may further be a multi-core processor having two or more independent processors (sometimes referred to as “cores”) that may execute instructions contemporaneously.

Promoter Region As used herein, “promoter region” refers to a DNA sequence recognized by the synthetic machinery of the cell, or introduced synthetic machinery, required to initiate the specific transcription of a gene.

Quantitative Measures: As used herein, “quantitative measures” refers to an absolute or relative measure. A quantitative measure can be, without limitation, a number, a statistical measurement (e.g., frequency, mean, median, standard deviation, or quantile), or a degree or a relative quantity (e.g., high, medium, and low). A quantitative measure can be a ratio of two quantitative measures. A quantitative measure can be a linear combination of quantitative measures. A quantitative measure may be a normalized measure.

Reference Sequence: As used herein, “reference sequence” refers to a known sequence used for purposes of comparison with experimentally determined sequences. For example, a known sequence can be an entire genome, a chromosome, or any segment thereof. A reference sequence can include at least about 20, at least about 50, at least about 100, at least about 200, at least about 250, at least about 300, at least about 350, at least about 400, at least about 450, at least about 500, at least about 1000, or more nucleotides. A reference sequence can align with a single contiguous sequence of a genome or chromosome or can include non-contiguous segments that align with different regions of a genome or chromosome. Example reference sequences, include, for example, human genome reference sequences, such as, hG19 and hG38.

Sample: As used herein, “sample” means anything capable of being analyzed by the methods and/or systems disclosed herein.

Sensitivity. As used herein, “sensitivity” means the probability of detecting the presence of a single nucleotide variant, an insertion, and a deletion at a given MAF and coverage and the probability of detecting the presence of a copy number variant at a given tumor fraction and coverage.

Sequencing: As used herein, “sequencing” refers to any of a number of technologies used to determine the sequence (e.g., the identity and order of monomer units) of a biomolecule, e.g., a nucleic acid such as DNA or RNA. Example sequencing methods include, but are not limited to, targeted sequencing, single molecule real-time sequencing, exon or exome sequencing, intron sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLID™ sequencing, MS-PET sequencing, and a combination thereof. In some implementations, sequencing can be performer by a gene analyzer such as, for example, gene analyzers commercially available from Illumina, Inc., Pacific Biosciences, Inc., or Applied Biosystems/Thermo Fisher Scientific, among many others.

Single Nucleotide Variant: As used herein, “single nucleotide variant” or “SNV” means a mutation or variation in a single nucleotide that occurs at a specific position in the genome.

Somatic Mutation: As used herein, “somatic mutation” means a mutation in the genome that occurs after conception. Somatic mutations can occur in any cell of the body except germ cells and accordingly, are not passed on to progeny.

Specifically binds: As used herein, “specifically binds” in the context of a probe or other oligonucleotide and a target sequence means that under appropriate hybridization conditions, the oligonucleotide or probe hybridizes to its target sequence, or replicates thereof, to form a stable probe: target hybrid, while at the same time formation of stable probe: non-target hybrids is minimized. Thus, a probe hybridizes to a target sequence or replicate thereof to a sufficiently greater extent than to a non-target sequence, to enable capture or detection of the target sequence. Appropriate hybridization conditions are well-known in the art, may be predicted based on sequence composition, or can be determined by using routine testing methods (see, e.g., Sambrook et al., Molecular Cloning, A Laboratory Manual, 2nd ed. (Cold Spring Harbor Laboratory Press, Cold Spring Harbor, NY, 1989) at §§ 1.90-1.91, 7.37-7.57, 9.47-9.51 and 11.47-11.57, particularly §§ 9.50-9.51, 11.12-11.13, 11.45-11.47 and 11.55-11.57, incorporated by reference herein).

Subject. As used herein, “subject” refers to an animal, such as a mammalian species (e.g., human) or avian (e.g., bird) species, or other organism, such as a plant. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.”

For example, a subject can be an individual who has been diagnosed with having a cancer, is going to receive a cancer therapy, and/or has received at least one cancer therapy. The subject can be in remission of a cancer. As another example, the subject can be an individual who is diagnosed of having an autoimmune disease. As another example, the subject can be a female individual who is pregnant or who is planning on getting pregnant, who may have been diagnosed of or suspected of having a disease, e.g., a cancer, an auto-immune disease.

Target Region: As used herein, “target region” refers to a genomic region of interest. For example, the genomic region of interest can correspond to one or more mutations that are consistent with one or more types of cancer. Additionally, the genomic region of interest can be enriched by one or more probes.

Threshold: As used herein, “threshold” refers to a predetermined value used to characterize experimentally determined values of the same parameter for different samples depending on their relation to the threshold.

Tumor Fraction: As used herein, “tumor fraction” refers to the estimate of the fraction of nucleic acid molecules derived from a tumor in a given sample. For example, the tumor fraction of a sample can be a measure derived from the max MAF of the sample or pattern of sequencing coverage of the sample or length of the cfDNA fragments in the sample or any other selected feature of the sample. In some instances, the tumor fraction of a sample is equal to the max MAF of the sample.

Variant: As used herein, a “variant” can be referred to as an allele. A variant is usually presented at a frequency of 50% (0.5) or 100% (1), depending on whether the allele is heterozygous or homozygous. For example, germline variants are inherited and usually have a frequency of 0.5 or 1. Somatic variants; however, are acquired variants and usually have a frequency of <0.5. Major and minor alleles of a genetic locus refer to nucleic acids harboring the locus in which the locus is occupied by a nucleotide of a reference sequence, and a variant nucleotide different than the reference sequence respectively. Measurements at a locus can take the form of allelic fractions (AFs), which measure the frequency with which an allele is observed in a sample.

### DETAILED DESCRIPTION

Cancer is usually caused by the accumulation of mutations within genes of an individual's cells, at least some of which result in improperly regulated cell division. Such mutations can include single nucleotide variations (SNVs), gene fusions, insertions, deletions, transversions, translocations, and inversions. These mutations can also include copy number variations that correspond to an increase or a decrease in the number of copies of a gene within a tumor genome relative to an individual's noncancerous cells. An extent of mutations present in cell-free nucleic acids and an amount of mutated cell-free nucleic acids of a sample can be used as biomarkers to determine tumor progression, predict patient outcome, and refine treatment choices. In various examples, the extent of mutations present in cell-free nucleic acids can be indicated by tumor cells copy number and tumor fraction for a given sample.

Additionally, cancer can be indicated by non-sequence modifications, such as methylation. Examples of methylation changes in cancer include local gains of DNA methylation in the CpG islands at the TSS of genes involved in normal growth control, DNA repair, cell cycle regulation, and/or cell differentiation. This increased amount of methylation can be associated with an aberrant loss of transcriptional capacity of involved genes and occurs at least as frequently as point mutations and deletions as a cause of altered gene expression.

Thus, DNA methylation profiling can be used to detect aberrant methylation in DNA of a sample. The DNA can correspond to certain genomic regions (“differentially methylated regions” or “DMRs”) that are normally hypermethylated or hypomethylated in a given sample type (e.g., cfDNA from the bloodstream) but which may show an abnormal degree of methylation that correlates to a neoplasm or cancer, e.g., because of unusually increased contributions of tissues to the type of sample (e.g., due to increased shedding of DNA in or around the neoplasm or cancer) and/or from extents of methylation of the genome that are altered during development or that are perturbed by disease, for example, cancer or any cancer-associated disease.

Deficiencies in homologous recombination repair pathways can be determined by identifying mutations in genes that are involved in the regulation of the homologous recombination repair pathways. For example, somatic mutations in genes that have previously been identified as being related to the regulation of homologous recombination repair pathways can be identified by analyzing mutations present in cell-free nucleic acids obtained from subjects. The accuracy of existing techniques to detect somatic mutations in cell-free nucleic acids of subjects is typically less than desired. In at least some scenarios, homologous recombination repair deficiencies can be characterized by deletions present in genes that regulate the homologous recombination repair pathways. The sensitivity of existing techniques for detecting the deletions that correspond to genes involved in the regulation of the homologous recombination repair pathways is somewhat limited.

The methods and systems described herein are directed to determining subjects having homologous recombination repair deficiencies by analyzing methylation data obtained from samples including cell-free nucleic acids of the subjects. The methylation data can indicate amounts of nucleic acid molecules having methylated cytosines in a number of genomic regions. To illustrate, the methylation data can correspond to genomic regions that are differentially methylated in subjects in which cancer is present. In addition, the methylation data can correspond to genomic regions that have been previously identified as having one or more mutations present in individuals in which cancer is present. The analysis of methylation data to determine subjects in which homologous recombination repair deficiencies are present improves the accuracy of the detection of homologous recombination repair deficiencies in relation to the accuracy achieved using existing techniques.

In one or more implementations, one or more computational models can be generated that determine a status of subjects with respect to homologous recombination repair deficiencies. The one or more computational models can implement at least one of one or more machine learning techniques or one or more statistical techniques to determine the status of subjects with respect to homologous recombination repair deficiencies. In various examples, the one or more computational models can analyze sequencing data that corresponds to samples obtained from subjects to determine the status of the subjects with respect to homologous recombination repair deficiencies. The sequencing data can indicate nucleic acid molecules that have at least one of a greater than expected number of methylated cytosines or fewer than expected methylated cytosines in a number of genomic regions. The number of genomic regions can exhibit one or more mutations in individuals in which one or more forms of cancer are present and/or in individuals in which homologous recombination repair deficiencies are present. The number of genomic regions can also include differentially methylated regions in individuals in which homologous recombination repair deficiencies are present.

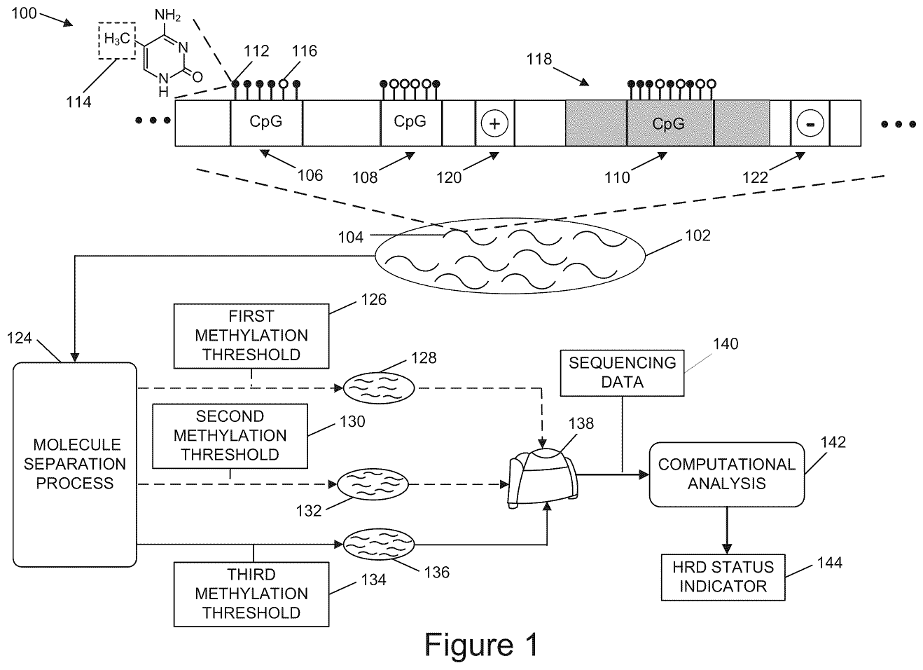

FIG. 1 is a diagrammatic representation of an example environment 100 that partitions nucleic acids based on methylation of cytosine molecules in a number of genomic regions of a reference sequence, according to one or more implementations. In one or more examples, the disease under consideration is a type of cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic (CLL), chronic myeloid (CML), chronic myelomonocytic (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

The environment 100 can include a sample 102. The sample 102 can be derived from fluid obtained from a subject. For example, the sample 102 can be derived from blood obtained from a subject. In one or more additional examples, the sample 102 can be derived from tissue of a subject. In various examples, the sample 102 can be derived from multiple sources. To illustrate, the sample 102 can be derived from one or more fluids of a subject and from tissue of a subject. In one or more illustrative examples, the subject can be a mammal. In one or more additional illustrative examples, the subject can be a human. In one or more further illustrative examples, the subject can be a non-human mammal.

The sample 102 can include a number of nucleic acids 104. Individual nucleic acids 104 can include a number of regions that have at least a threshold number of cytosine molecules and guanine molecules. In one or more examples, individual nucleic acids 104 can include regions having at least a threshold number of cytosine-guanine pairs. In various examples, at least a portion of the cytosine-guanine pairs included in the regions can be sequentially located in sequences of the nucleic acids 104. In one or more illustrative examples, a region of a nucleic acid having at least a threshold amount of cytosine-guanine pairs can be referred to herein as a “CG region” or a “CpG region.” In one or more examples, a CG region can include at least 200 base pairs. In one or more illustrative examples, a CG region can include from 200 base pairs to 5000 base pairs, from 300 base pairs to 3000 base pairs, from 200 base pairs to 2500 base pairs, or from 500 base pairs to 1500 base pairs. Additionally, a CG region can have a GC percentage of at least 50% and an observed-to-expected CpG ratio of at least 60%. The observed-to-expected CpG ratio can be calculated where the observed CpG is the number of CpGs identified in a given genomic region and the expected CpGs is the number of cytosines multiplied by the number of guanines divided by the number of bases in the genomic region. The expected CpGs can also be calculated by:

((number of cytosines+number of guanines)/2)/length of genomic region.

For example, a CG region can be determined using the techniques described by Gardiner-Garden M, Frommer M (1987). “CpG islands in vertebrate genomes”. Journal of Molecular Biology. 196 (2): 261-282. and/or Saxonov S, Berg P, Brutlag D L (2006). “A genome-wide analysis of CpG dinucleotides in the human genome distinguishes two distinct classes of promoters “. Proc Natl Acad Sci USA. 103 (5): 1412-1417.

In the illustrative example of FIG. 1, a portion of a sequence of an example nucleic acid 104 can include a first CG region 106, a second CG region 108, and a third CG region 110. Although the illustrative example of FIG. 1 illustrates a portion of a sequence of a nucleic acid 104 having three CG regions, nucleic acids 104 included in the sample 102 can have a different number of CG regions. For example, individual nucleic acids 104 included in the sample 102 can include at least 1 CG region, at least 5 CG regions, at least 10 CG regions, at least 25 CG regions, at least 50 CG regions, at least 100 CG regions, at least 250 CG regions, at least 500 CG regions, or at least 1000 CG regions.

Individual CG regions can include a number of molecules with a methylated cytosine. In the illustrative example of FIG. 1, the first CG region 106 can include a molecule with a methylated cytosine 112. In the illustrative example of FIG. 1, the molecule with a methylated cytosine 112 is 5-methylcytosine. Individual CG regions can also include a number of unmethylated cytosines. For example, the CG region 106 can include a molecule with an unmethylated cytosine 116. In various examples, at least a portion of the CG regions of a nucleic acid 104 can correspond to classification regions of a reference genome. In the illustrative example of FIG. 1, the first CG region 106, the second CG region 108, and the third classification region 110 can be classification regions. Classification regions can correspond to genomic regions of a reference genome that correspond to one or more mutations that are consistent with one or more biological conditions, such as one or more forms of cancer. The one or more mutations can include at least one of somatic mutations or germline mutations.

In at least some examples, the classification regions can correspond to genomic regions that are enriched as part of a diagnostic assay. In various examples, the classification regions can be differentially methylated regions located within a genomic region that corresponds to deficiencies in homologous recombination repair pathways. To illustrate, a gene 118 can correspond to a genomic region related to homologous recombination repair deficiencies. In one or more additional examples, the classification regions can be determined by identifying differentially methylated regions located in at least a portion of a gene that corresponds to homologous recombination repair deficiencies, where the differentially methylated regions are present in subjects in which homologous recombination repair deficiencies are present and where the differentially methylated regions are not present in subjects in which homologous recombination repair deficiencies are not present.

In addition to a number of CG regions, individual nucleic acids 104 can correspond to one or more positive control regions of a reference genome, such as positive control region 120. The positive control region 120 can include at least a threshold number of methylated cytosines in genomic regions of nucleic acids derived from cells that are obtained from subjects that are free of cancer and are also methylated in genomic regions of nucleic acids derived from cells that are obtained from subjects in which cancer is present. In various examples, the positive control region 120 can be hypermethylated in nucleic acids derived from cells obtained from subjects that are free of cancer and also in nucleic acids derived from cells obtained from subjects in which cancer is present. Individual nucleic acids 104 can also include one or more negative control regions, such as negative control region 122. The negative control region 122 can include less than a threshold number of methylated cytosines in nucleic acids derived from cells that are obtained from subjects that are free of cancer and also subjects in which cancer is not present. In one or more illustrative examples, the negative control region 122 can be hypomethylated in subjects that are free of cancer and also in subjects in which cancer is present. In various examples, the positive control regions and the negative control regions can be used to perform normalization calculations. The normalization calculations can be performed to generate input data for one or more computational models that are implemented to determine a homologous recombination repair deficiency for a given sample 102.

A molecule separation process 124 can be performed. The molecule separation process 124 can separate nucleic acids 104 included in the sample 102 based on an amount of methylation of cytosines of the individual nucleic acids 104. In one or more examples, the molecule separation process 124 can separate nucleic acids 104 included in the sample 102 based on amounts of methylation of cytosines included in CG regions of individual nucleic acids 104. In various examples, the molecule separation process 124 can separate the nucleic acids 104 into a plurality of groups with individual groups corresponding to respective amounts of methylation of cytosines of the nucleic acids 104.

In the illustrative example of FIG. 1, the molecule separation process 124 can be performed in relation to a first methylation threshold 126. Performing the molecule separation process 124 with regard to the first methylation threshold 126 can produce a first partition of nucleic acids 128. In one or more examples, the first methylation threshold 126 can indicate a first threshold number of methylated cytosines located in CG regions of the nucleic acids 104. The molecule separation process 124 can identify a number of nucleic acids 104 having fewer methylated cytosines in CG regions than the first methylation threshold 126. In various examples, the first methylation threshold 126 can correspond to a first methylation rate.

The molecule separation process 124 can also be performed with respect to a second methylation threshold 130. The second methylation threshold 130 can indicate an amount of methylation of cytosines in one or more genomic regions of the nucleic acids 104 that is greater than the amount of methylation of cytosines in the one or more regions corresponding to the first methylation threshold 126. The second methylation threshold 130 can indicate a number of methylated cytosines per a given number of nucleic acids. In one or more additional examples, the second methylation threshold 130 can correspond to a rate of methylation of nucleic acids that is greater than the rate of methylation that corresponds to the first methylation threshold 126. Performing the molecule separation process 124 with respect to the second methylation threshold 130 can produce a second partition of nucleic acids 132. In one or more examples, the molecule separation process 124 can identify nucleic acids 104 having a greater amount of methylation of cytosines than the first methylation threshold 126 and having a lower amount of methylation of cytosines than the second methylation threshold 130 to produce the second partition of nucleic acids 132.

Additionally, the molecule separation process 124 can also be performed with respect to a third methylation threshold 134. The third methylation threshold 134 can indicate an amount of methylation of cytosines in one or more genomic regions of the nucleic acids 104 that is greater than the amount of methylation of cytosines in the one or more regions corresponding to the first methylation threshold 126 and greater than the amount of methylation of cytosines in the one or more regions corresponding to the second methylation threshold 130. The third methylation threshold 134 can indicate a number of molecules with a methylated cytosine per a given number of nucleic acids. In one or more additional examples, the third methylation threshold 134 can correspond to a rate of methylation of cytosines that is greater than the rate of methylation that corresponds to the first methylation threshold 126 and greater than the rate of methylation that corresponds to the second methylation threshold 130. Performing the molecule separation process 124 with respect to the third methylation threshold 134 can produce a third partition of nucleic acids 136. In one or more examples, the molecule separation process 124 can identify nucleic acids 104 having a greater amount of methylation of cytosines than nucleic acids 104 included in the second partition of nucleic acids 132. In this way, the amount of methylation of cytosines of nucleic acids included in the first partition 128, the second partition 132, and the third partition 136 increases from the first partition 128 to the second partition 132 and increases from the second partition 132 to the third partition 136. In one or more illustrative examples, the first partition of nucleic acids 128 can be referred to as a hypomethylation partition, the second partition of nucleic acids 132 can be referred to as an intermediate partition, and the third partition of nucleic acids 136 can be referred to as a hypermethylation partition.

In one or more examples, the amount of methylation of cytosines of nucleic acids can correspond to a strength of binding to methyl binding domain (MBD). In these scenarios, the first partition 128, the second partition 132, and the third partition 134 can be produced based on different strengths of binding to MBD for nucleotides having different amounts of methylation of cytosines. In one or more examples, the molecule separation process 124 can include a series of washes where the nucleic acids 104 are contacted with solutions having different concentrations of sodium chloride (NaCl).

Partitioning of the nucleic acids can be performed by contacting the nucleic acids with a modified nucleotide specific binding reagent, such as a MBD of an MBP. A modified nucleotide specific binding reagent can bind to 5-methylcytosine (5mC). The modified nucleotide specific binding reagent, such as an MBD, can be coupled to paramagnetic beads, such as Dynabeads® M-280 Streptavidin via a biotin linker. Partitioning into fractions with different extents of methylation can be performed by increasing the NaCl concentration in a series of washes. The sequences eluted from the modified nucleotide specific binding reagent are partitioned into two or more fractions (e.g., hypo, hyper) depending on which wash (e.g., NaCl concentration) eluted the sequences. Resulting partitions can include one or more of the following nucleic acid forms: double-stranded DNA (dsDNA), shorter DNA fragments and longer DNA fragments.

The binding of the nucleic acids with the modified nucleotide specific binding reagent can be a function of number of methylated (or modified) sites per molecule, with molecules having more methylation eluting under increased salt concentrations. To elute the DNA into distinct populations based on the extent of methylation, a series of elution buffers of increasing NaCl concentration can be used. Salt concentrations can, in one or more implementations, range from about 100 nm to about 2500 mM NaCl. In various implementations, the molecule separation process 124 results in three (3) partitions. Molecules are contacted with a solution at a first salt concentration and comprising a molecule comprising a methyl binding domain, which molecule can be attached to a capture moiety, such as streptavidin. At the first salt concentration a population of molecules will bind to the MBD and a population will remain unbound. The unbound population can be separated as a “hypomethylated” population (hypo partition). For example, the first partition 128 can be representative of the hypomethylated form of DNA that remains unbound at a low salt concentration. In one or more illustrative examples, the concentration of NaCl of the solution used to produce the first partition 128 can be about 100 nM, about 120 nM, about 140 nM, about 160 nM, about 180 nM, about 200 nM. or about 250 nM.

The second partition 132 can be referred to as an “intermediate partition” and can be representative of intermediate methylation of CG regions of DNA that is eluted using an intermediate salt concentration, e.g., between 100 mM and 2000 mM concentration. In one or more additional illustrative examples, the concentration of NaCl of the solution used to produce the second partition 132 can be from about 100 mM to about 500 mM, from about 100 mM to about 1000 mM, from about 100 mM to about 1500 mM, from about 250 mM to about 1000 mM, from about 250 mM to about 1500 mM, from about 500 mM to about 1500 mM, from about 250 mM to about 2000 mM, from about 500 mM to about 2000 mM, or from about 1000 mM to about 2000 mM. The third partition 136 can be representative of hypermethylated forms of nucleic acids (hyper partition) and is eluted using a high salt concentration, e.g., at least about 2000 mM. In one or more further illustrative examples, the concentration of NaCl of the solution used to produce the third partition 136 can be from about 2000 mM to about 5000 mM, from about 2000 mM to about 4000 mM, from about 2000 mM to about 3500 mM, from about 2000 mM to about 3000 mM, or from about 2500 mM to about 4000 mM.

In various examples, the first partition 128 can correspond to a first range of binding strengths of nucleic acids to MBD and to a first amount of methylated cytosines in CG regions and the second partition 132 can correspond to a second range of binding strengths of nucleic acids to MBD and to a second amount of methylated cytosines in CG regions. The first range of binding strengths can be less than the second range of binding strengths. In one or more scenarios, a first solution having a first NaCl concentration can separate a first group of nucleic acids having the first range of binding strengths from MBD and a second solution having a second NaCl concentration can separate a second group of nucleic acids having the second range of binding strengths from MBD with the second NaCl concentration being greater than the first NaCl concentration. Additionally, the third partition 136 can correspond to a third range of binding strengths and a third amount of methylated cytosines in CG regions. The third range of binding strengths can be greater than the first range of binding strengths and greater than the second range of binding strengths. In one or more instances, a third solution having a third NaCl concentration can separate a third group of nucleic acids having the third range of binding strengths. The third NaCl concentration can be greater than the first NaCl concentration and greater than the second NaCl concentration.

In one or more illustrative examples, a plurality of nucleic acids derived from at least one of blood or tissue of a subject can be combined with a solution including an amount of MBD to produce a nucleic acid-MBD solution. A first wash of the nucleic acid-MBD solution can be performed with a first washing solution including a first NaCl concentration to produce a first nucleic acid fraction and a first residual solution. The first nucleic acid fraction can include a first portion of the plurality of nucleic acids and the first residual solution can include a second portion of the plurality of nucleic acids. In one or more examples, the first portion of the plurality of nucleic acids can have a first range of binding energies to MBD that are less than a second range of binding energies to MBD of the second portion of the plurality of nucleic acids.

Additionally, a second wash of the first residual solution can be performed with a second washing solution including a second concentration of NaCl that is greater than the first concentration of NaCl to produce a second nucleic acid fraction and a second residual solution. The second nucleic acid fraction can include a first subset of the second portion of the plurality of nucleic acids and the second residual solution can include a second subset of the second portion of the plurality of nucleic acids. The first subset of the second portion of the plurality of nucleic acids can have a third range of binding energies to MBD that are less than a fourth range of binding energies to MBD of the second subset of the second portion of the plurality of nucleic acids. In various examples, the second range of binding energies can comprise the third range of binding energies and the fourth range of binding energies. Further, a third wash of the second residual solution can be performed with a third solution including a third concentration of NaCl that is greater than the second concentration of NaCl to produce a third nucleic acid fraction that includes the second subset of the second portion of the plurality of nucleic acids.

Subsequent to the first wash, the second wash, and the third wash a determination can be made that the first nucleic acid fraction is associated with the first partition 128. A first molecular barcode can then be attached to the first portion of the plurality of nucleic acids with the first molecular barcode indicating the first partition 128. In this way, a sequencing read that corresponds to the first partition 128 can be identified based on determining that the sequencing read includes the first molecular barcode. In addition, a determination can be made that the second nucleic acid fraction is associated with the second partition 132 of the plurality of partitions. In these situations, a second molecular barcode can be attached to the first subset of the second portion of the plurality of nucleic acids with the second molecular barcode indicating the second partition 132. As a result, a sequencing read that corresponds to the second partition 132 can be identified based on determining that the sequencing read includes the second molecular barcode. Further, a determination can be made that the third nucleic acid fraction is associated with the third partition 136. A third molecular barcode can then be attached to the second subset of the second portion of the plurality of nucleic acids with the third molecular barcode indicating the third partition 136. In these instances, a sequencing read that corresponds to the third partition 136 can be identified based on determining that the sequencing read includes the third molecular barcode.

In one or more additional examples, the molecule separation process 124 can include performing one or more sodium bisulfite sequencing processes to determine amounts of methylation of the nucleic acids 102. In one or more illustrative examples, sodium bisulfite sequencing can be performed according to Li Y, Tollefsbol T O. DNA methylation detection: bisulfite genomic sequencing analysis. Methods Mol Biol. 2011; 791:11-21. doi: 10.1007/978-1-61779-316-5_2. PMID: 21913068; PMCID: PMC3233226. In one or more further examples, at least one of the first partition of nucleic acids 128, the second partition of nucleic acids 132, or the third partition of nucleic acids 136 can be subjected to an additional separation process. For example, the one or more molecule separation processes 124 can include digestion of at least one of the first partition of nucleic acids 128, the second partition of nucleic acids 132, or the third partition of nucleic acids 136 using methyl sensitive restriction enzyme (MSRE). Digestion of the nucleic acids included in the first partition 128, the second partition 132, and/or the third partition 136 with MSRE can result in separation of the nucleic acids included in one of the first partition 126, the second partition 132, or the third partition 136 that do not have levels of methylation corresponding to the respective first methylation threshold 126, the second methylation threshold 130, or the third methylation threshold 134. Digestion of nucleic acids included in at least one of the first partition 128, the second partition 132, or the third partition 136 using MSRE can increase the amount of nucleic acids included in the first partition 128 having amounts of methylation no greater than the first methylation threshold 126, increase the amount of nucleic acids included in the second partition 132 having amounts of methylation between the second methylation threshold 130 and the first methylation threshold 126, and/or increase the amount of nucleic acids included in the third partition 136 having amounts of methylation between the third methylation threshold 134 and the second methylation threshold 132.

The environment 100 can include a sequencing machine 138. In one or more examples, the sequencing machine 138 can be any of a number of sequencing machines that can perform one or more sequencing operations that amplify nucleic acids present in a sample 104. In various examples, the sequencing machine 138 can perform next-generation sequencing operations.

In the illustrative example of FIG. 1, the separation of the nucleic acids 102 into the first partition 128 and the second partition 132 can be optional as indicated by the dotted lines between the molecule separation process 124 and the first partition 128 and the second partition 132. That is, in at least some examples, only the third methylation threshold 134 is used to produce the third partition 136 that corresponds to the hypermethylated nucleic acids and the third partition 136 is then provided to the sequencing machine 138. Any remaining nucleic acids that are not included in the third partition 136 are not provided to the sequencing machine 138. Additionally, in these scenarios, the third partition 136 is subjected to MSRE digestion and any remaining nucleic acids that are not included in the third partition are not subjected to MSRE digestion. Further, in these scenarios, the nucleic acids included in the third partition 136 are subjected to blunt-end ligation.

In one or more additional illustrative examples, the separation of the nucleic acids into the second partition 132 is optional and the molecule separation process 124 using the first methylation threshold 126 and the third methylation threshold 130 results in producing the first partition 128 corresponding to hypomethylated nucleic acids and the third partition 136 corresponding to hypermethylated nucleic acids. In these scenarios, the nucleic acids included in the first partition 128 and the third partition 132 are provided to the sequencing machine 136 and the nucleic acids corresponding to the second partition 132 are not provided to the sequencing machine 138. Further, in various examples, the molecule separation process 124 can result in two partitions: a partition that combines the nucleic acids from the first partition 128 and the second partition 132 and an additional partition that includes the nucleic acids of the third partition 136.

Prior to sequencing, blunt-end ligation can be performed on the extracted polynucleotides and adapters, as well as the addition of tags (e.g., molecular barcodes) to the extracted polynucleotides. The extracted polynucleotides can also be enriched by causing hybridization between the extracted polynucleotides and probes that correspond to classification regions of a reference sequence. The enrichment process can identify thousands, hundreds of thousands, up to millions of polynucleotides that correspond to classification regions associated with the probes. In one or more examples, the enrichment process can be performed in relation to genomic regions that are part of a diagnostic assay. In these instances, the genomic regions can correspond to nucleotide sequences of a reference genome that indicate the presence of one or more forms of cancer. In one or more additional examples, the enrichment process can be performed in relation to genomic regions in which mutations can result in deficiencies of homologous recombination repair mechanisms. In these scenarios, the genomic regions may comprise a gene that corresponds to homologous recombination repair pathways. Thousands, up to millions of unenriched polynucleotides that correspond to non-classification regions of the reference sequence can also be present after the enrichment process.

Subsequent to the enrichment process, the enriched polynucleotides can be amplified according to one or more amplification processes. The one or more amplification processes can produce thousands, up to millions of copies of individual enriched polynucleotides. In one or more examples, a portion of the unenriched polynucleotides can be amplified, in some instances, but not to the extent that the enriched polynucleotides are amplified. The one or more amplification processes can generate an amplification product that undergoes one or more sequencing operations. After performing one or more sequencing operations with respect to the sample 104, the sequencing machine 138 can produce sequencing data 140.

The sequencing data 140 can include alphanumeric representations of the nucleic acids included in an amplification product generated by the sequencing machine 140. For example, the sequencing data 140 can include, for individual nucleic acids of the amplification product, data that corresponds to a string of letters that represent the respective chains of nucleotides that correspond to the individual nucleic acids.

The sequencing data 140 can be stored in one or more data files. For example, the sequencing data 140 can be stored in a FASTQ file that comprises a text-based sequencing data file format storing raw sequence data and quality scores. In one or more additional examples, the sequencing data 142 can be stored in a data file according to a binary base call (BCL) sequence file format. In one or more further examples, the sequencing data 142 can be stored in a BAM file. In one or more examples, the sequencing data 140 can comprise at least about one gigabyte (GB), at least about 2 GB, at least about 3 GB, at least about 4 GB, at least about 5 GB, at least about 8 GB, or at least about 10 GB. An individual sequence representation included in the sequencing data 140 can be referred to herein as a “read” or a “sequencing read.” In various examples, individual first nucleic acids included in the sample 102 can correspond to many sequence representations included in the sequencing data 140 as a result of the amplification of the individual first nucleic acids. In one or more additional examples, individual second nucleic acids included in the sample 102 can correspond to a single sequence representation or a few sequence representations included in the sequencing data 140 as a result of the absence of amplification of the individual second nucleic acids.

The environment 100 can also include performing a computational analysis 142 based on the sequencing data 140. The computational analysis 142 can include analyzing sequence reads that corresponds to one or more of the partitions 128, 132, 136 to determine a homologous recombination deficiency (HRD) status indicator 144. The HRD status indicator 144 can correspond to a probability of a homologous recombination repair deficiency being present in a subject. In various examples, the computational analysis 142 can implement at least one of one or more machine learning techniques or one or more statistical techniques to generate the HRD status indicator 144.

In one or more examples, the computational analysis 142 can include determining an amount of sequencing reads having amounts of methylation that correspond to at least one of the first methylation threshold 126, the second methylation threshold 130, or the third methylation threshold 134 and that correspond to one or more classification regions, such as at least one of the first CG region 106, the second CG region 108, or the third CG region 110. In one or more illustrative examples, the computational analysis 142 can include determining a number of sequence reads included in the sequencing data 140 that correspond to the nucleic acids included in the third partition 136. In various examples, the computational analysis 142 can implement one or more computational models that include components that correspond to a portion of the classification regions. For example, a training process can be performed to identify a subset of the classification regions that are predictive of HRD status. The subset of classification regions can correspond to one or more components of the one or more computational models implemented as part of the computational analysis 142 to generate the HRD status indicator 144.

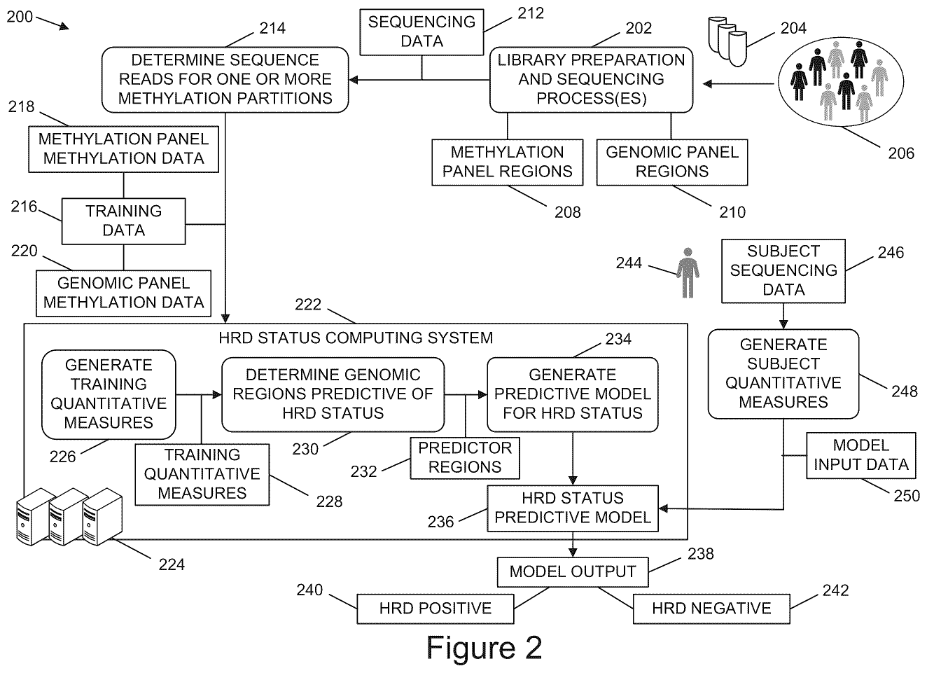

FIG. 2 is a diagrammatic representation of an example framework 200 to determine homologous recombination deficiency status using one or more computational models and based on methylation status of cell free nucleic acid molecules, according to one or more implementations. The framework 200 can include performing one or more library preparation and sequencing processes 202. The one or more library preparation and sequencing processes 202 can be performed in relation to a number of samples 204. The number of samples 204 can be obtaining from subjects 206. In one or more illustrative examples, a first portion of the subjects 206 can be free of homologous recombination repair deficiencies. Additionally, homologous recombination repair deficiencies can be present in a second portion of the subjects 206. The subjects 206 can be training subjects from which data is generated to train one or more computational models to determine the status of subjects with respect to homologous recombination deficiencies.

In one or more examples, the first portion of the subjects 206 can be determined based on the absence of one or more mutations with respect to genomic regions that are related to homologous recombination repair. In one or more additional examples, the second portion of the subjects 206 can be determined based on the presence of the one or more mutations with respect to the genomic regions that are related to homologous recombination repair. In one or more illustrative examples, the one or more mutations with respect to genomic regions related to homologous recombination repair can include at least one of germline deletions, germline rearrangements, germline fusions, somatic deletions, somatic rearrangements, somatic fusions, or homozygous deletions. In various examples, deletions present in the second portion of the subjects 206 having homologous recombination deficiencies can include single nucleotide variants or indels. In one or more further examples, the second portion of the subjects 206 in which a homologous recombination deficiency is present can be determined based on responsiveness to inhibitors of poly-adenosine diphosphate (ADP) ribose polymerase (PARP). Individuals in which homologous recombination deficiencies are present tend to be responsive to treatment with PARP inhibitors. (See Keung M Y T, Wu Y, Vadgama J V. PARP Inhibitors as a Therapeutic Agent for Homologous Recombination Deficiency in Breast Cancers. J Clin Med. 2019 Mar. 30; 8(4):435. doi: 10.3390/jcm8040435. PMID: 30934991; PMCID: PMC6517993). As a result, a portion of the subjects 206 in which a tumor is present and that have a decrease in tumor cells in response to treatment of the tumor with one or more PARP inhibitors can be included in the second portion of the subjects 206.

The library preparation and sequencing processes 202 can include the extraction of nucleic acid molecules from the samples 204. In one or more implementations, the nucleic acid molecules comprise cell-free nucleic acids (e.g., cell-free DNA). In various implementations, the samples 204 can include one or more samples selected from one or more of blood, plasma, serum, urine, fecal, saliva samples, combinations thereof, and/or the like. In one or more additional examples, the samples 204 can comprise one or more samples selected from one or more of whole blood, a blood fraction, a tissue biopsy, pleural fluid, pericardial fluid, cerebrospinal fluid, and peritoneal fluid.

The extraction of nucleic acid molecules from the samples 204 can include implementing one or more cell lysis techniques to cleave the membranes of cells included in the samples 204 and applying one or more proteases to break down proteins included in the samples 204. The extraction of nucleic acid molecules from the samples 204 can also include a number of washing and/or elution techniques to separate the nucleic acid molecules from other components included in the samples 204. In various examples, thousands, up to millions, up to billions of nucleic acid molecules can be extracted from the samples 204.

The one or more library preparation and sequencing processes 202 can include one or more separation processes that correspond to separating nucleic acid molecules into a number of partitions based on the characteristics of the nucleic acid molecules. Examples of characteristics that can be used for partitioning nucleic acid molecules include multiple different nucleotide modifications, methylation level, nucleosome binding, sequence mismatch, immunoprecipitation, and/or proteins that bind to DNA. In one or more illustrative examples, a heterogeneous population of nucleic acid molecules can be partitioned into nucleic acid molecules with one or more epigenetic modifications and without the one or more epigenetic modifications. Examples of epigenetic modifications include, but are not limited to, presence or absence of methylation; level of methylation, hydroxymethylation, and type of methylation (5′ cytosine or 6 methyladenine).

In one or more examples, the nucleic acid molecules extracted from samples 204 can include nucleic acids having varying levels of methylation. Methylation can occur from any one or more post-replication or transcriptional modifications. Post-replication modifications include modifications of the nucleotide cytosine, including, but not limited to, 5-methylcytosine, 5-hydroxymethylcytosine, 5-formylcytosine and 5-carboxylcytosine. The one or more library preparation and sequencing processes 202 can separate nucleic acid molecules extracted from samples 204 into a number of partitions with individual partitions corresponding to different levels of methylation. For example, the one or more library preparation and sequencing processes 202 can produce a first partition of nucleic acid molecules having first levels of methylation, a second partition of nucleic acid molecules having second levels of methylation, and a third partition of nucleic acid molecules having third levels of methylation. In various examples, the second levels of methylation can be greater than the first levels of methylation and the third levels of methylation can be greater than the first levels of methylation and the second levels of methylation. In one or more illustrative examples, the one or more library preparation and sequencing processes 202 can include the molecule separation process 124 described with respect to FIG. 1.

In at least some examples, molecular barcodes can be added to nucleic acids that correspond to one or more methylation partitions. For example, one or more first molecular barcodes can be added to nucleic acids having a first level of methylation and included in a first methylation partition, one or more second molecular barcodes can be added to nucleic acids having a second level of methylation and included in a second methylation partition, and one or more third molecular barcodes can be added to nucleic acids having a third level of methylation and included in a third methylation partition. In scenarios where nucleic acids included in the samples 204 are subjected to one or more enrichment processes, molecular barcodes can be added to nucleic acids being enriched.

The library preparation and sequencing processes 202 can also include one or more enrichment processes. The one or more enrichment processes can amplify the number of nucleic acids included in the samples 204 having one or more specified sequences. In various examples, the nucleic acids included in the samples 204 can be enriched with respect to methylation panel regions 208. The methylation panel regions 208 can corresponds to genomic regions of a reference genome that are differentially methylated in subjects having one or more biological conditions. For example, the methylation panel regions 208 can include one or more genomic regions that are differentially methylated involved in subjects in which one or more homologous recombination repair deficiencies are present. In one or more additional examples, the methylation panel regions 208 can include one or more genomic regions that are differentially methylated in individuals in which one or more forms of cancer are present. In at least some examples, the methylation panel regions 208 can be the subject of one or more diagnostic assays.

One or more enrichment processes included in the library preparation and sequencing processes 202 can also be performed with respect to genomic panel regions 210. The genomic panel regions 210 can include one or more portions of a reference genome that are the subject of one or more diagnostic assays. For example, the genomic panel regions 210 can correspond to a number of genomic regions in which at least one of somatic mutations or germline mutations are present in individuals in which a biological condition is present. In one or more illustrative examples, the genomic panel regions 210 can correspond to a number of genomic regions in which at least one of one or more somatic mutations or one or more germline mutations are present in individuals in which one or more forms of cancer are present. In one or more additional illustrative examples, the genomic panel regions 210 can include driver mutations that correspond to one or more forms of cancer. In various examples, one or more of the methylation panel regions 208 can include or overlap with at least a portion of one or more genomic panel regions 210.

In various examples, the library preparation and sequencing processes 202 can include performing one or more enrichment processes with respect to nucleic acids having one or more amounts of methylation. For example, the library preparation and sequencing processes 202 can include performing one or more enrichment processes with respect to nucleic acids having genomic regions with at least a threshold amount of methylation. In one or more examples, the library preparation and sequencing processes 202 can include performing one or more enrichment processes with respect to nucleic acids having at least one hypermethylated genomic region. In one or more illustrative examples, the library preparation and sequencing processes 202 can include performing one or more enrichment processes with respect to nucleic acids having at least a threshold amount of methylation in one or more CG regions that correspond to at least one of the methylation panel regions 208 or the genomic panel regions 210.

The library preparation and sequencing processes 202 can include performing one or more amplification processes and one or more sequencing processes to generate sequencing data 212. The sequencing data 212 can include alphanumeric representations of the nucleic acids included in an amplification product generated by the one or more library preparation and sequencing processes 202. For example, the sequencing data 212 can include, for individual nucleic acids of the amplification product, data that corresponds to a string of letters that represent the respective chains of nucleotides that correspond to the individual nucleic acids. The sequencing data 212 can be stored in one or more data files.

The framework 200 can also include, at operation 214, determining sequence reads for one or more methylation partitions. In one or more examples, the sequencing data 212 can be analyzed to determine sequence reads that correspond to nucleic acids having at least one CG region that corresponds to at least one methylation partition. In one or more illustrative examples, determining sequence reads for one or more methylation partitions at operation 214 can include determining sequence reads included in the sequencing data 212 that correspond to a hyper methylation partition. In at least some examples, determining sequence reads that correspond to at least one methylation partition can include analyzing the sequencing data 212 to determine sequence reads having one or more molecular barcodes that correspond to the at least one methylation partition.

Determining sequence reads included in the sequencing data 212 that correspond to one or more methylation partitions at operation 214 can generate training data 216. The training data 216 can be used to train at least one of one or more machine learning models or one or more statistical models to determine homologous recombination deficiency status of subjects. The training data 216 can include methylation panel methylation data 218. The methylation panel methylation data 218 can include sequence reads included in the sequencing data 212 that correspond to nucleic acids included in one or more methylation partitions and that correspond to the methylation panel regions 208. In various examples, the sequence reads of the sequencing data 212 that correspond to at least one methylation partition can be further analyzed to determine a subset of sequence reads that correspond to the at least one methylation partition and that also correspond to the methylation panel regions 208. In one or more illustrative examples, the methylation panel methylation data 218 can include sequence reads that correspond to the methylation panel regions 208 and that have at least a threshold amount of methylated cytosines in CG regions of the methylation panel regions 208. For example, the sequencing data 212 can be analyzed at operation 214 to produce the methylation panel methylation data 218 such that the methylation panel methylation data 218 includes sequence reads that correspond to a hyper methylation partition and that correspond to the methylation panel regions 208. In one or more examples, the methylation panel methylation data 218 can be determined by analyzing the sequencing data 212 to determine sequence reads that include one or more molecular barcodes that correspond to the methylation panel regions 208. In one or more additional examples, the methylation panel methylation data 218 can be determined by analyzing the sequencing data 212 to determine sequence reads having at least a threshold amount of homology with the methylation panel regions 208.

The training data 216 can also include genomic panel methylation data 220. The genomic panel methylation data 220 can include sequence reads included in the sequencing data 212 that correspond to nucleic acids included in one or more methylation partitions and that correspond to the genomic panel regions 210. In one or more examples, the sequence reads of the sequencing data 212 that correspond to at least one methylation partition can be further analyzed to determine an additional subset of sequence reads that correspond to the at least one methylation partition and that also correspond to the genomic panel regions 210. In one or more illustrative examples, the genomic panel methylation data 220 can include sequence reads that correspond to the genomic panel regions 210 and have less than an additional threshold amount of methylated cytosines in CG regions of the genomic panel regions 210. To illustrate, the sequencing data 212 can be analyzed at operation 214 to produce the screening panel methylation data 220 such that the screening panel methylation data 220 includes sequence reads that correspond to a hypo methylation partition and that correspond to the genomic panel regions 210. In one or more examples, the genomic panel methylation data 220 can be determined by analyzing the sequencing data 212 to determine sequence reads that include one or more molecular barcodes that correspond to the genomic panel regions 210. In one or more additional examples, the genomic panel methylation data 220 can be determined by analyzing the sequencing data 212 to determine sequence reads having at least a threshold amount of homology with the genomic panel regions 210.

One or more alignment processes can be performed to generate the training data 216. For example, one or more alignment processes can be performed to determine an amount of homology between sequence reads included in the sequencing data 212 and the methylation panel regions 208 to determine the methylation panel methylation data 218. In addition, one or more alignment processes can be performed to determine an amount of homology between sequence reads included in the sequencing data 212 and the genomic panel regions 210 to determine the genomic panel methylation data 220. Further, one or more alignment processes can be performed to determine an amount of homology between sequence reads included in the sequencing data 212 and one or more molecular barcodes. The one or more molecular barcodes can correspond to at least one of one or more methylation partitions, the methylation panel regions 208, or the genomic panel regions 210.

The amount of homology between a given sequence read and one or more genomic regions of a reference sequence can indicate a number of positions of the reference sequence that have the same nucleotide as corresponding positions of the given sequence read. A sequence read can be aligned with a genomic region of a reference sequence based on determining that the sequence read and the genomic region of the reference sequence have at least a threshold amount of homology. In scenarios where a sequence read has at least the threshold amount of homology with respect to multiple genomic regions of the reference sequence, the genomic region of the reference sequence having the greatest amount of homology with the sequence read can be determined to be aligned with the sequence read.

The amount of homology between a given sequence read and a portion of a reference sequence can be determined using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Genetics Computer Group, University Research Park, Madison Wis.), using default settings, which uses the algorithm of Needleman and Wunsch (J. Mol. Biol. 48; 443-453 (1970)). The amount of homology between a sequence read and a portion of the reference sequence can also be determined using a Burrows-Wheeler aligner (Li, H., & Durbin, R. (2009). Fast and accurate short read alignment with Burrows-Wheeler transform. Bioinformatics, 25(14), 1754-1760).

In one or more illustrative examples, the sequencing data 212 can be analyzed to determine a first group of sequence reads that correspond to a hyper methylated partition. The first group of sequence reads can also be analyzed to determine a first subset of the first group of sequence reads that corresponds to the methylation panel regions 208 and a second subset of the first group of sequence reads that corresponds to the genomic panel regions 210. In these scenarios, the training data 216 can include sequence reads that correspond to nucleic acids having at least a threshold amount of methylation related to the hyper methylation partition and that also correspond to the methylation panel regions 208 and the genomic panel regions 210.

In one or more additional illustrative examples, the sequencing data 212 can be analyzed to determine a second group of sequence reads that correspond to a hypo methylated partition. The second group of sequence reads can also be analyzed to determine a first subset of the second group of sequence reads that correspond to the methylation panel regions 208 and a second subset of the second group of sequence reads that corresponds to the genomic panel regions 210. In these situations, the training data 216 can include sequence reads that correspond to nucleic acids having at least a threshold amount of methylation related to the hyper methylation partition and having no greater than an additional threshold amount of methylation related to the hypo methylation partition and that also correspond to the methylation panel regions 208 and the genomic panel regions 210.

In at least some illustrative examples, the training data 216 can include both the first group of sequence reads that correspond to the hyper methylated partition and the second group of sequence reads that correspond to the hypo methylated partition. In these instances, the training data 216 can include sequence reads that correspond to first nucleic acids having at least a threshold amount of methylation related to the hyper methylation partition and second nucleic acids having no greater than an additional threshold amount of methylation related to the hypo methylated partitions and that also correspond to the methylation panel regions 208 and the genomic panel regions 210.

In various examples, at least one of tumor fraction or tumor cells copy number can be generated for a given sample 204 based on portions of the sequencing data 212 that corresponds to the given sample 204. In one or more illustrative examples, tumor fraction and/or tumor cells copy number can be calculated as described in U.S. patent application Ser. No. 17/691,049 filed Mar. 9, 2022, which is incorporated herein in its entirety. A subset of the samples 204 that have at least one of a threshold tumor fraction or a threshold tumor cells copy number can be determined. In various examples, the sequence reads included in the training data 216 can be derived from the subset of the samples 204 having at least the threshold tumor fraction and/or the threshold tumor cells copy number.

The architecture 200 can include an HRD status computing system 222 that obtains the training data 216 and analyzes the training data 216 to generate one or more models to determine an HRD status of subjects. The HRD status computing system 222 can include one or more computing devices 224. The one or more computing devices 224 can include at least one of one or more desktop computing devices, one or more mobile computing devices, or one or more server computing device. In various examples, at least a portion of the one or more computing devices 224 can be included in a remote computing environment, such as a cloud computing environment. In one or more examples, the library preparation and sequencing processes 202, determining sequence reads for one or more methylation partitions at operation 214, and the operations performed by the computing system 224 can be performed by a single entity. In one or more additional examples, the library preparation and sequencing processes 202, determining sequence reads for one or more methylation partitions at operation 214, and the operations performed by the computing system 224 can be performed by multiple organizations.

At operation 226, the HRD status computing system 222 can generate training quantitative measures 228. The training quantitative measures 228 can correspond to a number of sequence representations that correspond to sequence reads included in the training data 216 and that correspond to individual classification regions of a reference sequence. In one or more examples, prior to determining the training quantitative measures 228, the HRD status computing system 222 can identify one or more groups of sequence representations. For example, individual sequence representations can correspond to individual sequencing reads that are included in the sequencing data 212. In these scenarios, sequence representations can include multiple reads that correspond to a single nucleic acid molecule included in the samples 204. In one or more additional examples, the sequence representations can correspond to individual nucleic acid molecules included in the samples 204. In these situations, the HRD status computing system 222 can determine a group of reads included in the training data 216 that correspond to an individual nucleic acid molecules included in the samples 204 based on molecular barcodes that are common to each group of sequencing reads. That is, individual nucleic acid molecules included in the samples 204 can be encoded with molecular barcodes that uniquely identify the individual nucleic acid molecules and, in at least some cases, the individual nucleic acid molecules can be represented by multiple sequencing reads included in the training data 216. Accordingly, when multiple sequence representations are present in the training data 216 that correspond to a single nucleic acid molecule included in the samples 204, the HRD status computing system 222 can group the multiple sequence representations together. In various examples, the groups of sequence representations that correspond to a single nucleic acid molecule included in the samples 204 can be referred to herein as “families.” Additionally, start and stop positions with respect to the reference sequence of the sequence representations having a common molecular barcode can be used to group the sequence representations that correspond to individual nucleic acids included in the samples 204. In one or more illustrative examples, an individual sequence representation that represents a family of sequence representations that corresponds to a single nucleic acid molecule included in the samples 204 can be referred to herein as a “consensus sequence representation.”

The one or more classification regions can correspond to genomic regions of a reference sequence that have an amount of methylation in cfDNA obtained from subjects in which a homologous recombination repair deficiency is present relative to an amount of methylation of the genomic regions in cfDNA obtained from subjects in which a homologous recombination repair deficiency is not present. The one or more classification regions can also include at least a threshold amount of cytosine-guanine content. In various examples, the one or more classification regions can include a series of cytosine-guanine (CG) pairs in the 5′→3′ direction (CpG sites), such as at least 3 CpG sites, at least 5 CpG sites, at least 8 CpG sites, at least 10 CpG sites, at least 12 CpG sites, at least 15 CpG sites, at least 18 CpG sites, or at least 20 CpG sites. In one or more illustrative examples, the training quantitative measures 228 can indicate a number of sequence representations derived from the training data 216 that correspond to individual genomic panel regions 210. Additionally, the one or more classification regions can correspond to genomic regions of a reference sequence that include at least one of one or more germline mutations or one or more somatic mutations in individuals in which a homologous recombination repair deficiency is present and/or genomic regions that correspond to differentially methylated regions in individuals in which at least one of an HRD is present or one or more forms of cancer are present. In one or more additional illustrative examples, the training quantitative measures 228 can indicate a number of sequence representations derived from the training data 226 that correspond to individual methylation panel regions 208. In at least some examples, the training quantitative measures 228 can indicate a number of sequence representations derived from the training data 216 that correspond to individual methylation panel regions 208 and a number of sequence representations derived from the training data 216 that correspond to individual genomic panel regions 210.

In various examples, the training quantitative measures 228 can include normalized quantitative measures. The normalized quantitative measures can be determined by analyzing the number of sequence representations derived from the training data 216 that correspond to a classification region in relation to the number of sequence representations derived from the training data 216 that correspond to one or more control regions. In one or more examples, the normalized quantitative measures can be determined by analyzing the number of sequence representations derived from the training data 216 that correspond to a classification region in relation to at least one of a first number of sequence representations derived from the training data 216 that correspond to one or more positive control regions or a second number of sequence representations derived from the training data 216 that correspond to one or more negative control regions. A positive control region can comprise a genomic region of a reference sequence having at least a threshold amount of sequence representations that correspond to nucleic acids with a methylated cytosine and including at least a threshold number of CpG sites. A positive control region can have at least the threshold amount of sequence representations that correspond to nucleic acids with a methylated cytosine in samples obtained from subjects in which a homologous recombination repair deficiency is present and in samples obtained from subjects in which a homologous recombination repair deficiency is not present. In one or more examples, a negative control region can comprise a genomic region of a reference sequence having less than a threshold amount of sequence representations that correspond to nucleic acids with a methylated cytosine and at least a threshold number of CpG sites. A negative control region can have less than a threshold amount of sequence representations that correspond to nucleic acids with methylated cytosines in samples obtained from subjects in which a homologous recombination repair deficiency is present and in samples obtained from subjects in which a homologous recombination repair deficiency is not present.

The training quantitative measures 228 can also be normalized with respect to guanine-cytosine (G-C) content. For example, for individual methylation panel regions 208 and/or individual genomic panel regions 210, G-C content can be determined that indicates a number of guanine nucleotides and a number of cytosine nucleotides of sequence representations that correspond to the individual methylation panel regions 208 and/or the individual genomic panel regions 210. In addition, frequency of G-C content can be determined for a partition of G-C content of a plurality of partitions. Individual partitions of G-C content can correspond to different ranges of values of G-C content. In this way, the frequency of G-C content for a given methylation panel region 208 or a given genomic panel region 210 can be represented by a G-C content distribution for individual methylation panel regions 208 and/or individual genomic panel regions 210. An expected amount of coverage for individual methylation panel regions 208 and/or individual genomic panel regions 210 can be determined based on the frequency of G-C content for the methylation panel regions 208 and/or the genomic panel regions 210. At least a portion of the normalized training quantitative measures can include G-C normalized coverage data that is determined based on the expected amount of coverage for individual HRD genomic regions 208 and/or individuals screening panel regions 210.

At operation 230, the HRD status computing system 222 can analyze the training quantitative measures 228 to generate predictor regions 232. The predictor regions 232 can indicate genomic regions included in at least one of the methylation panel regions 208 or the genomic panel regions 210 that include methylation patterns that are indicative of homologous recombination repair deficiencies. For example, the HRD status computing system 222 can analyze the training quantitative measures 228 to determine CG regions having differing amounts of methylation in subjects in which homologous recombination repair deficiencies are present in relation to subjects in which homologous recombination repair deficiencies are not present. In one or more examples, at least a portion of the predictor regions 232 can include genomic regions that correspond to a subset of at least one of the methylation panel regions 208 or the genomic panel regions 210 in which CG regions have a greater amount of methylation in subjects in which homologous recombination repair deficiencies are present in relation to subjects in which homologous recombination repair deficiencies are not present. In one or more additional examples, at least a portion of the predictor regions 232 can include genomic regions that correspond to a subset of at least one of the methylation panel regions 208 or the genomic panel regions 210 in which CG regions have less methylation in subjects in which a homologous recombination repair deficiency is present in relation to subjects in which a homologous recombination repair deficiency is not present.

The HRD status computing system 222 can implement at least one of one or more machine learning techniques or one or more statistical techniques to determine the predictor regions 232. In one or more examples, the HRD status computing system 222 can implement one or more logistic regression models to determine the predictor regions 232. In one or more illustrative examples, the HRD status computing system 222 can implement one or more elastic net linear regression algorithms to determine the predictor regions 232. In one or more additional illustrative examples, the HRD status computing system 222 can implement one or more lasso regression techniques to determine the predictor regions 232. In various examples, the HRD status computing system 222 can analyze quantitative measures that correspond to hundreds of genomic regions, up to thousands of genomic regions, up to tens of thousands of genomic regions to determine the predictor regions 232.

At operation 234, the HRD status computing system 222 can use the predictor regions to generate a predictive model for HRD status, such as an HRD status predictor model 236. The HRD status predictor model 236 can include individual components that correspond to the individual predictor regions 232. For example, the HRD status predictor model 236 can include individual variables having individual weights that correspond to the individual predictor regions 232. The HRD status predictive model 236 can generate a model output 238 having at least two outcomes: HRD positive 240 or HRD negative 242. In one or more examples, the model output 238 generated using the HRD status predictive model 238 can correspond to a probability of a given sample being derived from a subject in which a homologous recombination repair deficiency is present. The HRD status computing system 222 can analyze the probability of a given sample being derived from a subject in which a homologous recombination repair deficiency is present in relation to a threshold probability to determine whether the model output 238 is HRD positive 240 or HRD negative 242.

In one or more illustrative examples, after the HRD status predictive model 236 is generated by the HRD status computing system 222 using the training quantitative measures 228, the HRD status predictive model 236 can be used to determine HRD status of an additional subject 244 that is not included in the training subjects 206. For example, subject sequencing data 246 can be generated using one or more samples derived from the additional subject 244. To illustrate, the subject sequencing data 246 can be generated from one or more cfDNA samples derived from the additional subject 244. In various examples, the subject sequencing data 246 can be generated according to processes that are similar or the same as the one or more library preparation and sequencing processes 202. The subject sequencing data 246 can include sequence representations that correspond to nucleotide sequences of nucleic acids included in one or more samples derived from the additional subject 244. In at least some examples, the sequence representations included in the subject sequencing data 246 can correspond to sequence reads generated by one or more library preparation and sequencing processes.

The subject sequencing data 246 can be analyzed to generate subject quantitative measures at operation 248. In one or more examples, operation 248 can be performed by the HRD status computing system 222. The subject quantitative measures generated at operation 248 can be included in model input data 250. The model input data 250 can be provided to the HRD status predictive model 236 to determine an HRD status of the additional subject 244. In one or more illustrative examples, the model input data 250 can include normalized quantitative metrics that correspond to the predictor regions 232. For example, at operation 248, a number of sequence representations that correspond to nucleic acids included in one or more samples derived from the additional subject 244 having at least a threshold amount of methylation can be determined. In at least some examples, at operation 248, sequence representations that correspond to nucleic acids that correspond to the hyper methylation partition can be identified. Additionally, at operation 248, at least one of a number of sequence representations or a number of nucleic acids that are related to one or more samples obtained from the additional subject 244 and that correspond to the predictor regions 232 can be determined. Further, one or more normalization procedures can be performed to generate normalized quantitative measures that are determined based on sequence representations derived from the subject sequencing data 246 that correspond to at least a portion of the predictor regions 232. In various examples, the model input data 250 can include an input vector representing the normalized quantitative measures determined using the subject sequencing data 246.

Based on the model input data 250, the HRD status predictive model 236 can determine a model output 238 for the additional subject 244. The model output 238 can indicate a probability that a homologous recombination repair deficiency is present in the additional subject 244. The model output 238 can indicate that the additional subject 244 corresponds to an HRD positive 240 status or an HRD negative 242 status. In one or more illustrative examples, the model output 238 can be used to determine that the additional subject 244 has an HRD positive status 240 based on the probability of a homologous recombination repair deficiency being present in the additional subject 244 being at least a threshold probability. In one or more examples, the model output 238 can be used to determine one or more treatment recommendations for the additional subject 244. For example, in scenarios where the model output 238 for the additional subject 244 is HRD positive 240, a treatment for the additional subject 244 can include one or more PARP inhibitors.

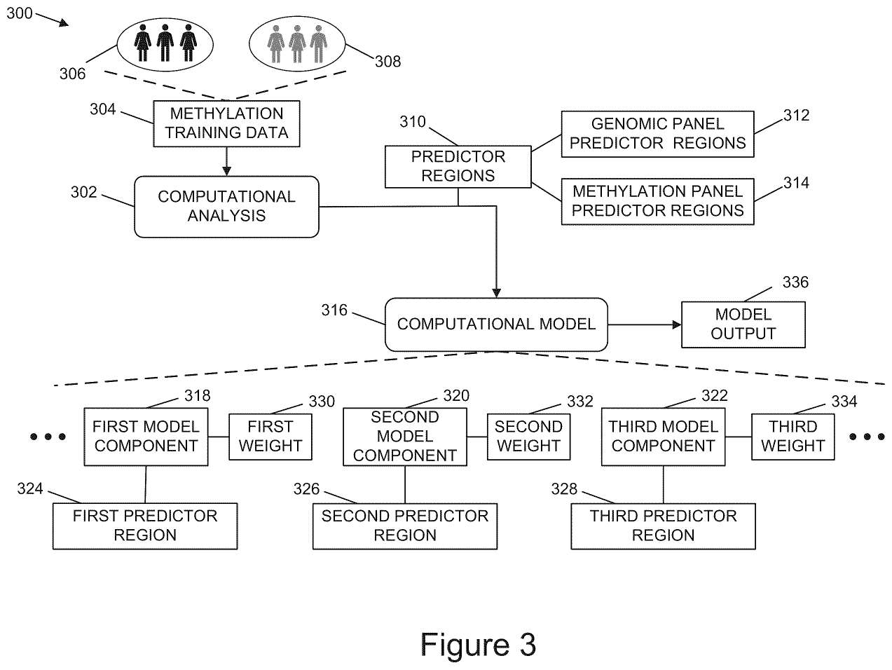

FIG. 3 is a diagrammatic representation of an example framework 300 to generate one or more computational models to determine homologous recombination deficiency status of a subject, in accordance with one or more implementations. In at least some examples, operations described in relation to the framework 300 can be performed by the HRD status computing system 200 described with respect to FIG. 2. The framework 300 can include a computational analysis 302 that is performed with respect to methylation training data 304. The methylation training data 304 can be derived from a first group of training subjects 306 in which a homologous recombination repair deficiency is not present and a second group of training subjects 308 in which a homologous recombination repair deficiency is not present.

The methylation training data 304 can include sequence representations of nucleic acids derived from samples obtained from the first group of training subjects 306 and the second group of training subjects 308. The sequence representations can correspond to polynucleotide molecules or sequence reads derived from samples obtained from the first group of training subjects 306 and the second group of training subjects 308. The methylation training data 304 can also include sequence representations that correspond to nucleic acids having at least a threshold amount of methylation in CG regions of one or more classification regions. For example, the methylation training data 304 can include sequence representations that correspond to nucleic acids included in a hyper methylation partition with respect to one or more CG regions of at least one classification region. In one or more additional examples, the methylation training data 304 can include sequence representations that correspond to nucleic acids included in a hypo methylation partition with respect to one or more CG regions of at least one classification region. The one or more classification regions can correspond to genomic regions of a reference genome that can include one or more genomic mutations in individuals in which one or more biological conditions are present. In at least some examples, at least a portion of the classification regions can be differentially methylated in individuals in which one or more biological conditions are present.

The computational analysis 302 can include analyzing the methylation training data 304 to determine a number of predictor regions 310. The predictor regions 310 can include a subset of the classification regions. In one or more examples, the predictor regions 310 can be differentially methylated in individuals in which a homologous recombination repair deficiency is present in relation to individuals in which a homologous recombination repair deficiency is not present. In one or more additional examples, the predictor regions 310 can be differentially methylated in individuals in which one or more forms of cancer are present. In various examples, the predictor regions can include CG regions having a threshold number of methylated cytosines. For example, at least a portion of the predictor regions 310 can be included in a hypermethylated partition. Additionally, the predictor regions 310 can include CG regions having no greater than an additional number of methylated cytosines. To illustrate, at least a portion of the predictor regions 310 can be included in a hypomethylated partition.

In various examples, the predictor regions 310 can include genomic panel predictor regions 312. The genomic panel HRD predictor regions 312 can include genomic regions that are part of a screening panel. The screening panel can include a diagnostic process to identify the presence of genomic mutations that can be indicative of one or more biological conditions. In one or more examples, the screening panel can include a diagnostic process to identify the presence of genomic mutations that can be indicative of one or more forms of cancer. In one or more illustrative examples, the genomic panel predictor regions 312 can include genomic regions that correspond to one or more genomic regions that include at least one of one or more somatic mutations or one or more germline mutations that are indicative of one or more forms of cancer Further, the genomic panel predictor regions 312 can be predictive of subjects in which a homologous recombination repair deficiency is present.

The predictor regions 310 can also include methylation panel predictor regions 314. The methylation panel predictor regions 314 can include genomic regions that are differentially methylated in individuals in which a homologous recombination repair deficiency is present. In addition, the methylation panel predictor regions 314 can include portions of one or more genes that are differentially methylated in individuals in which a homologous recombination repair deficiency one or more forms of cancer are present. In various examples, the computational analysis 302 include analyzing portions of genes that are differentially methylated in the second group of training subjects 308 in relation to the first group of training subjects 306.

The computational analysis 302 can include generating a model for individual classification regions, where the model generates an indicator of HRD status of subjects. In one or more examples, the indicator of HRD status of subjects can include a probability of one or more subjects having a homologous recombination repair deficiency. In various examples, the computational analysis 302 can generate a logistic regression model for individual classification regions to determine an indicator of HRD status of subjects. In at least some examples, the models generated for the individual classification regions can have input data that includes quantitative measures that correspond to the individual classification regions and that indicates a form of cancer for which one or more mutations are present in the individual classification regions in subjects in which the form of cancer is present.

The quantitative measures can include metrics indicating a number of sequence representations that correspond to the individual classification regions and that correspond to at least one methylation partition. The number of sequence representations can correspond to a number of sequence reads derived from nucleic acids included in one or more samples obtained from the first group of training subjects 306 and the second group of training subjects 308 that correspond to the individual classification regions or a number of nucleic acids present in one or more samples obtained from the first group of training subjects 306 and the second group of training subjects 308 that correspond to the individual classification regions. In one or more examples, the quantitative measures can include normalized quantitative measures. The normalized quantitative measures can include a ratio of counts of sequence representations derived from samples obtained from the first group of training subjects 306 and the second group of training subjects 308 that correspond to individual classification regions in relation to counts of sequence representations derived from samples obtained from the first group of training subjects 306 and the second group of training subjects 308 that correspond to one or more control regions of a reference sequence. In various examples, the normalized quantitative measures can also be generated using a CG normalization process.

In one or more illustrative examples, the computational analysis 302 can include analyzing quantitative measures that correspond to at least 1000 genomic regions, at least 5000 genomic regions, at least 8000 genomic regions, at least 10,000 genomic regions, at least 12,000 genomic regions, at least 15,000 genomic regions, at least 18,000 genomic regions, at least 20,000 genomic regions, at least 25,000 genomic regions, or at least 30,000 genomic regions to determine the predictor regions 310. At least one of the panel HRD predictor regions 312 or the genomic HRD predictor regions 314 can include at least 25 genomic regions, at least 50 genomic regions, at least 75 genomic regions, at least 100 genomic regions, at least 150 genomic regions, at least 200 genomic regions, at least 250 genomic regions, at least 300 genomic regions, at least 350 genomic regions, at least 400 genomic regions, at least 450 genomic regions, or at least 500 genomic regions.

The analysis of quantitative measures for individual classification regions can generate p-values for individual classification regions. The individual p-values can indicate a measure of significance of individual classification regions in determining whether an HRD status indicator determined using a logistic regression model of the individual classification region accurately corresponds to the HRD status of individuals included in at least one of the first group of training subjects 306 or the second group of training subjects. In various examples, the samples obtained from the first group of training subjects 306 and the second group of training subjects 308 can be divided such that a first portion of samples obtained from the first group of training subjects 306 and the second group of training subjects 308 can used as training samples to generate the logistic regression model for individual classification regions. A second portion of samples obtained from the first group of training subjects 306 and the second group of training subjects 308 can be used as testing samples to determine the p-values for the individual classification regions.

The classification regions can be ordered according to the p-values that correspond to the individual classification regions. In at least some examples, the lower the p-value for an individual classification region, the greater the significance of the classification region in predicting HRD status of subjects. In one or more examples, the classification regions can be ranked from the classification regions having the lowest p-values to the classification regions having the highest p-values. In various examples, a subset of the classification regions can correspond to the predictor regions 310 according to the p-values of the predictor regions 310 and be selected for representation in a computational model 316. The classification regions selected for representation in the computational model 316 can include at least one of one or more genomic panel predictor regions 312 or one or more methylation panel predictor regions 314. In one or more illustrative examples, the 50 classification regions having the lowest p-values can be selected for representation in the computational model 316, the 100 classification regions having the lowest p-values can be selected for representation in the computational model 316, the 150 classification regions having the lowest p-values can be selected for representation in the computational model 316, the 200 classification regions having the lowest p-values can be selected for representation in the computational model 316, the 250 classification regions having the lowest p-values can be selected for representation in the computational model 316, the 300 classification regions having the lowest p-values can be selected for representation in the computational model 316, the 350 classifications regions having the lowest p-values can be selected for representation in the computational model 316, the 400 classification regions having the lowest p-values can be selected for representation in the computational model 316, the 450 classification regions having the lowest p-values can be selected for representation in the computational model 316, the 500 classification regions having the lowest p-values can be selected for representation in the computational model 316, the 750 classification regions having the lowest p-values can be selected for representation in the computational model 316, the 1000 classification regions having the lowest p-values can be selected for representation in the computational model 316, or the 1500 regions having the lowest p-values can be selected for representation in the computational model 316.

The computational model 316 can include a number of components. The components of the computational model 316 can include variables that can be predictive of the HRD status of a subject. In one or more examples, individual components of the computational model 316 can correspond to at least one of one or more predictor regions 310. In various examples, the individual components of the computational model 316 can be determined based on sequence representations included in the methylation training data 304 that correspond to a hypo methylation partition and the one or more genomic panel predictor regions 312. Additionally, the individual components of the computational model 316 can be determined based on sequence representations included in the methylation training data 304 that correspond to the hyper methylation partition and the one or more methylation panel predictor regions 314.

In the illustrative example of FIG. 3, the computational model 316 can include a number of components, such as a first model component 318, a second model component 320, and a third model component 322. In one or more examples, the first model component 318 can include one or more first functions, the second model component 320 can include one or more second functions, and the third model component 322 can include one or more third functions. The first model component 318 can correspond to a first predictor region 324, the second model component 320 can correspond to a second predictor region 326, and the third model component 322 can correspond to a third predictor region 328. Additionally, the components of the computational model 316 can correspond to at least one parameter that can indicate a measure of significance of the component in determining HRD status of subjects. For example, the components of the computational model 316 can correspond to one or more weights. To illustrate, the first model component 318 can have a first weight 330, the second model component 320 can have a second weight 332, and the third model component 322 can have a third weight 334.

The computational model 316 can be trained using quantitative measures determined using the methylation training data 304 with respect to the predictor regions 310. In one or more examples, sequence representations derived from the first group of training subjects 306 and the second group of training subjects 308 can be analyzed to determine amounts of methylation in one or more CG regions of the individual sequence representations. Additionally, sequence representations generated from samples obtained from the first group of training subjects 306 and the second group of training subjects 308 having genomic regions with at least a threshold amount of methylation in CG regions can be aligned with a reference genome. The aligned sequence representations can then be analyzed to determine an amount of homology between the sequence representations and the predictor regions 310. Counts of the aligned sequence representations that correspond to the predictor regions 310 can be determined. Further, normalized quantitative measures can be determined based on the counts of the aligned sequence representations that correspond to the predictor regions 310 and counts of sequence representations that correspond to at least one of one or more positive control regions or one or more negative control regions.

In at least some examples, the computational model 316 can include one or more machine learning models or one or more statistical models that are generated using normalized quantitative measures of the predictor regions 310. In one or more illustrative examples, the computational model 316 can include a logistic regression model that is trained and validated using normalized quantitative measures derived from samples obtained from the first group of training subjects 306 and the second group of training subjects 308 and that correspond to the predictor regions 310. In one or more examples, one or more least absolute shrinkage and selection operator (lasso) regression techniques can be used to generate the computational model 316 that includes a logistic regression model. In one or more additional examples, a training process for the computational model 316 can include performing one or more elastic regularization processes. In various examples, a training process for the computational model 316 can include optimizing one or more tuning parameters based on the normalized quantitative measures using one or more validation techniques to generate the computational model 316. The optimization of the tuning parameters can be performed to minimize overfitting of the training data to the computational model 316. In one or more additional illustrative examples, a training process for the computational model 316 can include a lambda optimization process to generate the computational model 316. In various examples, a lambda optimization process can determine one or more parameters that correspond to the components of the computational model 316. To illustrate, a lambda optimization process can determine the first weight 330 of the first model component 318, the second weight 332 of the second model component 320, and the third weight 334 of the third model component 322.

The computational model 316 can generate a model output 336. For one or more samples obtained from a given subject, the model output 336 can indicate a status of the subject with respect to a homologous recombination repair deficiency. To illustrate, the model output 336 can indicate a probability of a homologous recombination repair deficiency being present in an individual. In one or more illustrative examples, the model output 336 can include a probit value in relation to the status of a subject with respect to a homologous recombination repair deficiency being present in the subject.

In one or more examples, the computational model 316 can generate a model output 336 indicating an HRD status of individuals, where different forms of cancer may be present in the individuals. For example, the computational model 316 can determine probabilities of a homologous recombination repair deficiency being present in a first group of subjects in which a first form of cancer is present and probabilities of a homologous recombination repair deficiency being present in a second group of subjects in which a second form of cancer is present. In one or more additional examples, the computational model 316 can determine probabilities of a homologous recombination repair deficiency being present in subjects in which a specified form of cancer is present. To illustrate, the computational model 316 can determine probabilities of a homologous recombination repair deficiency being present in subjects in which colorectal cancer is present or probabilities of a homologous recombination repair deficiency being present in subjects n which prostate cancer is present. In at least some examples, the forms of cancer for which the computational model 316 can be used to determine HRD status of subjects can depend on the forms of cancer present in the first group of training subjects 306 and the second group of training subjects 308.

In various examples, the computational model 316 can use a threshold probability for the presence of a homologous recombination repair deficiency to determine an HRD status of one or more subjects. The threshold probability can be determined by analyzing the model output 336 with respect to subjects in which a homologous recombination repair deficiency is present and subjects in which a homologous recombination repair deficiency is not present. The threshold probability can correspond to a probability that indicates a model output 336 that captures a greatest number of subjects in which a homologous recombination repair deficiency is present. In at least some examples, the threshold probability used by the computational model 316 to determine whether a homologous recombination repair deficiency is present in a subject can be different for different forms of cancer.

In one or more examples, the computational model 316 can include additional components to generate the model output 336 that are in addition to the model components that correspond to the predictor regions 310. For example, the computational model 316 can include one or more first additional components that correspond to copy number variation in one or more genomic regions that are related to regulation of homologous recombination repair pathways. Additionally, the computational model 316 can include one or more second additional components that correspond to loss of heterozygosity in one or more genomic regions that are related to regulation of homologous recombination repair pathways. At least one of the first additional components or the second additional components of the computational model 316 can be used to generate the model output 336.

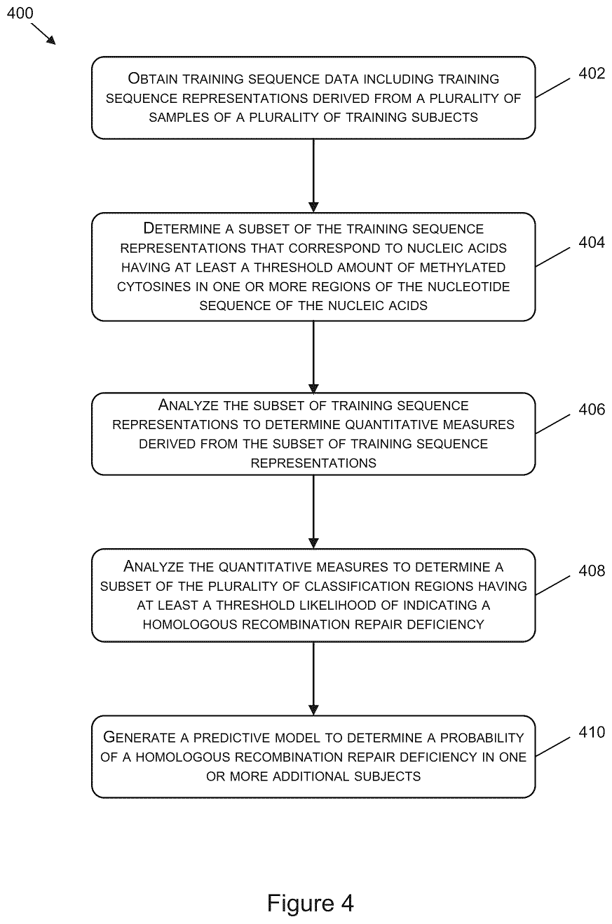

FIG. 4 is a flow diagram of an example process 400 to determine a probability of a homologous recombination repair deficiency being present in one or more subjects based on methylation data derived from a sample obtained from the one or more subjects, according to one or more implementations. At operation 402, the process 400 includes obtaining training sequence data including training sequence representations derived from a plurality of samples of a plurality of training subjects. The sequence representations can correspond to sequence reads generated by one or more sequencing machines. In these scenarios, the sequence reads can correspond to nucleic acids extracted from the plurality of samples obtained from the plurality of training subjects. In one or more additional examples, the sequence representations can indicate nucleic acids included in the plurality of samples. In various examples, individual sequence representations can correspond to a family of sequence reads that correspond to an individual nucleic acid included in the plurality of samples.

The process 400 can also include, at operation 404, determining a subset of the training sequence reads that correspond to nucleic acids having at least a threshold amount of methylated cytosines in one or more regions of the nucleotide sequences of the nucleic acids. Individual training sequencing representations can correspond to at least a portion of a nucleic acid derived from a sample of the plurality of samples having a CG region with a threshold amount of methylated cytosines. In one or more illustrative examples, the plurality of samples can include cell-free nucleic acids. In one or more examples, methylated cytosines can be determined using at least one of sodium bisulfite conversion and sequencing, Tet-assisted bisulfite sequencing (TAB-Seq), differential enzymatic cleavage, treatment with MSRE, or MBD partitioning. In one or more additional examples, methylated cytosines can be determined using one or more single molecule sequencing methods, such as nanopore DNA sequencing or those described in Eid, J., et al. (2009) “Real-time DNA sequencing from single polymerase molecules”. Science, 323(5910), 133-138.

At operation 406, the process 400 can include analyzing the subset of training sequence representations to determine quantitative measures derived from the subset of training sequence representations. The quantitative measures can indicate an amount of sequence representations that correspond to one or more genomic regions of a reference sequence. In one or more examples, the quantitative measures can indicate an amount of sequence representations that correspond to classification regions of a reference genome. The classification regions can include promoter regions that correspond to genomic regions that include one or more mutations present in individuals in which one or more forms of cancer are present. The classification regions can also include differentially methylated regions that include genomic regions having different amounts of methylation of cytosines in CG regions of individuals in which one or more forms of cancer are present in relation to additional amounts of methylation of cytosines in CG regions of individuals in which cancer is not detected. Additionally, the classification regions can include one or more genomic regions that are enriched as part of a screening panel that is used to identify individuals in which one or more forms of cancer are present. Further, the classification regions can include one or more genomic regions that include one or more mutations that are present in individuals in which a homologous recombination repair deficiency is present. In these scenarios, the classification regions can include at least one of at least a portion of the ATM gene, at least a portion of the BRCA1 gene, at least a portion of the BRCA2 gene, at least a portion of the CDK12 gene, at least a portion of the CHEK2 gene, at least a portion of the PALB2 gene, or at least a portion of the RAD51D gene.

The quantitative measures can also include normalized quantitative measures. The normalized quantitative measures can correspond to an amount of the subset of the training sequence representations having at least a threshold amount of methylated cytosines in CG regions that are included in classification regions in relation to an amount of the subset of the training sequence representations having at least the threshold amount of methylated cytosines in CG regions that are included in one or more control regions. For example, the normalized quantitative measures can indicate a ratio of counts of the subset of the training sequence representations having at least a threshold amount of methylated cytosines in CG regions that are included in classification regions in relation to counts of the subset of the training sequence representations having at least the threshold amount of methylated cytosines in CG regions that are included in the one or more control regions. The one or more control regions can include genomic regions of a reference sequence having at least the threshold amount of methylated cytosines in CG regions of individuals in which one or more forms of cancer are present and in additional individuals in which cancer is not present.

In addition, the process 400 can include, at operation 408, analyzing the quantitative measures to determine a subset of the plurality of classification regions having at least a threshold likelihood of indicating a homologous recombination repair deficiency. In one or more examples, one or more models can be generated for individual classification regions, where the models can be implemented to determine a status of subjects with respect to homologous recombination repair deficiencies. For example, at least one of one or more machine learning regression models or statistical regression models can be generated for the individual classification regions based on the quantitative measures for individual classification regions. A measure of significance of the individual classification regions can be determined based on the one or more models that correspond to the individual classification regions. In one or more illustrative examples, p-values can be calculated for the one or more models of the individual classification regions. The p-values can be used to rank the classification regions to indicate the measure of significance of the classification regions in identifying individuals in which a homologous recombination repair deficiency is present. The subset of the classification regions can be selected according to a number of classification regions having at least a threshold amount of significance in determining individuals in which a homologous recombination repair deficiency is present.

Further, at operation 410, the process 400 can include generating a predictive computational model to determine a probability of a homologous recombination repair deficiency being present in one or more additional subjects. The predictive computational model can include a number of components with individual components corresponding to at least one classification region of the subset of the plurality of classification regions. In one or more examples, the predictive computational model can be generated using one or more elastic regularization techniques to minimize overfitting of the predictive computational model to the data used to train the predictive model. In at least some examples, the predictive computational model can include a machine learning-based regression model or a statistical-based regression model. In one or more illustrative examples, the predictive computational model can include a logistic regression model. The predictive model can determine an HRD status indicator for subjects based on the probability of a homologous recombination deficiency being present in subjects. In one or more illustrative examples, the predictive computational model can generate an output indicating a positive HRD status for subjects in response to determining that a probability of a homologous recombination repair deficiency being present is at least a threshold probability. In one or more additional illustrative examples, the predictive computational model can generate an output indicating a negative HRD status for subjects in response to determining that a probability of a homologous recombination repair deficiency being present is less than the threshold probability.

In various examples, the sequence representations provided to the predictive computational model during the training process or after the training process have at least a threshold amount of methylation of cytosines in classification regions. The sequence representations that satisfy the methylation levels can be produced, at least in party, using one or more molecule separation processes. The molecule separation processes can include combining a plurality of nucleic acids derived from at least one of blood or tissue of a subject with a solution including an amount of methyl binding domain (MBD) proteins to produce a nucleic acid-MBD protein solution. A plurality of washes can then be performed of the nucleic acid-MBD protein solution with a salt solution to produce a number of nucleic acid fractions. Individual nucleic acid fractions can have a threshold number of molecules with a methylated cytosine in regions of the plurality of nucleic acids having at least the threshold cytosine-guanine content. In one or more illustrative examples, a wash of the plurality of washes can be performed with a solution having a concentration of sodium chloride (NaCl) and can produce a nucleic acid fraction of the number of nucleic acid fractions having a range of binding energies to MBD proteins.

In one or more examples, a first nucleic acid fraction can be determined is associated with a first partition of a plurality of partitions of nucleic acids. The first partition corresponding to a first range of binding energies to MBD proteins. Further, a first molecular barcode can be attached to nucleic acids of the first nucleic acid fraction. The first molecular barcode can be associated with the first partition. In addition, a second nucleic acid fraction can be determined that is associated with a second partition of the plurality of partitions of nucleic acids. The second partition can correspond to a second range of binding energies to MBD proteins different from the first range of binding energies to MBD proteins. A second molecular barcode can be attached to nucleic acids of the second nucleic acid fraction. The second molecular barcode being associated with the second partition.

### Samples

Isolation and extraction of cell free polynucleotides may be performed through collection of samples using a variety of techniques. A sample can be any biological sample isolated from a subject. Samples can include body tissues, whole blood, platelets, serum, plasma, stool, red blood cells, white blood cells or leucocytes, endothelial cells, tissue biopsies (e.g., biopsies from known or suspected solid tumors), cerebrospinal fluid, synovial fluid, lymphatic fluid, ascites fluid, interstitial or extracellular fluid (e.g., fluid from intercellular spaces), gingival fluid, crevicular fluid, bone marrow, pleural effusions, cerebrospinal fluid, saliva, mucous, sputum, semen, sweat, urine. Samples are preferably body fluids, particularly blood and fractions thereof, and urine. Such samples include nucleic acids shed from tumors. The nucleic acids can include DNA and RNA and can be in double and single-stranded forms. A sample can be in the form originally isolated from a subject or can have been subjected to further processing to remove or add components, such as cells, enrich for one component relative to another, or convert one form of nucleic acid to another, such as RNA to DNA or single-stranded nucleic acids to double-stranded. Thus, for example, a body fluid sample for analysis is plasma or serum containing cell-free nucleic acids, e.g., cell-free DNA (cfDNA).

In some implementations, the sample volume of body fluid taken from a subject depends on the desired read depth for sequenced regions. Example volumes are about 0.4-40 ml, about 5-20 ml, about 10-20 ml. For example, the volume can be about 0.5 ml, about 1 ml, about 5 ml, about 10 ml, about 20 ml, about 30 ml, about 40 ml, or more milliliters. A volume of sampled blood can be between about 5 ml to about 20 ml.

The sample can comprise various amounts of nucleic acid. The amount of nucleic acid in a given sample can be equated with multiple genome equivalents. For example, a sample of about 30 ng DNA can contain about 10,000 (10⁴) haploid human genome equivalents and, in the case of cfDNA, about 200 billion (2×10¹¹) individual polynucleotide molecules. Similarly, a sample of about 100 ng of DNA can contain about 30,000 haploid human genome equivalents and, in the case of cfDNA, about 600 billion individual molecules.

In some implementations, a sample comprises nucleic acids from different sources, e.g., from cells and from cell-free sources (e.g., blood samples, etc.). Typically, a sample includes nucleic acids carrying mutations. For example, a sample optionally comprises DNA carrying germline mutations and/or somatic mutations. Typically, a sample comprises DNA carrying cancer-associated mutations (e.g., cancer-associated somatic mutations). In some implementations of the present disclosure, cell free nucleic acids in a subject may derive from a tumor. For example, cell-free DNA isolated from a subject can comprise ctDNA.

Example amounts of cell-free nucleic acids in a sample before amplification typically range from about 1 femtogram (fg) to about 1 microgram (μg), e.g., about 1 picogram (pg) to about 200 nanogram (ng), about 1 ng to about 100 ng, about 10 ng to about 1000 ng. In some implementations, a sample includes up to about 600 ng, up to about 500 ng, up to about 400 ng, up to about 300 ng, up to about 200 ng, up to about 100 ng, up to about 50 ng, or up to about 20 ng of cell-free nucleic acid molecules. Optionally, the amount is at least about 1 fg, at least about 10 fg, at least about 100 fg, at least about 1 pg, at least about 10 pg, at least about 100 pg, at least about 1 ng, at least about 10 ng, at least about 100 ng, at least about 150 ng, or at least about 200 ng of cell-free nucleic acid molecules. In certain implementations, the amount is up to about 1 fg, about 10 fg, about 100 fg, about 1 pg, about 10 pg, about 100 pg, about 1 ng, about 10 ng, about 100 ng, about 150 ng, or about 200 ng of cell-free nucleic acid molecules. In some implementations, methods include obtaining between about 1 fg to about 200 ng cell-free nucleic acid molecules from samples.

Cell-free nucleic acids typically have a size distribution of between about 100 nucleotides in length and about 500 nucleotides in length, with molecules of about 110 nucleotides in length to about 230 nucleotides in length representing about 90% of molecules in the sample, with a mode of about 168 nucleotides length and a second minor peak in a range between about 240 to about 440 nucleotides in length. In certain implementations, cell-free nucleic acids are from about 160 to about 180 nucleotides in length, or from about 320 to about 360 nucleotides in length, or from about 440 to about 480 nucleotides in length.

In some implementations, cell-free nucleic acids are isolated from bodily fluids through a partitioning step in which cell-free nucleic acids, as found in solution, are separated from intact cells and other non-soluble components of the bodily fluid. In some of these implementations, partitioning includes techniques such as centrifugation or filtration. Alternatively, cells in bodily fluids are lysed, and cell-free and cellular nucleic acids processed together. Generally, after addition of buffers and wash steps, cell-free nucleic acids are precipitated with, for example, an alcohol. In certain implementations, additional clean up steps are used, such as silica-based columns to remove contaminants or salts. Non-specific bulk carrier nucleic acids, for example, are optionally added throughout the reaction to optimize certain aspects of the example procedure, such as yield. After such processing, samples typically include various forms of nucleic acids including double-stranded DNA, single-stranded DNA and/or single-stranded RNA. Optionally, single stranded DNA and/or single stranded RNA are converted to double stranded forms so that they are included in subsequent processing and analysis steps. Additional details regarding cfDNA partitioning and related analysis of epigenetic modifications that are optionally adapted for use in performing the methods disclosed herein are described in, for example, WO 2018/119452, filed Dec. 22, 2017, which is incorporated by reference.

### Nucleic Acid Tags

In certain implementations, tags providing molecular identifiers or barcodes are incorporated into or otherwise joined to adapters by chemical synthesis, ligation, or overlap extension PCR, among other methods. In some implementations, the assignment of unique or non-unique identifiers, or molecular barcodes in reactions follows methods and utilizes systems described in, for example, U.S. patent applications 20010053519, 20030152490, 20110160078, and U.S. Pat. Nos. 6,582,908, 7,537,898, and 9,598,731, which are each incorporated by reference.

Tags are linked (e.g., ligated) to sample nucleic acids randomly or non-randomly. In some implementations, tags are introduced at an expected ratio of identifiers (e.g., a combination of unique and/or non-unique barcodes) to microwells. For example, the identifiers may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In some implementations, the identifiers are loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In certain implementations, the average number of identifiers loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers per genome sample. The identifiers are generally unique or non-unique.

One example format uses from about 2 to about 1,000,000 different tags, or from about 5 to about 150 different tags, or from about 20 to about 50 different tags, ligated to both ends of a target nucleic acid molecule. For 20-50×20-50 tags, a total of 400-2500 tags are created. Such numbers of tags are typically sufficient for different molecules having the same start and stop points to have a high probability (e.g., at least 94%, 99.5%, 99.99%, 99.999%) of receiving different combinations of tags.

In some implementations, identifiers are predetermined, random, or semi-random sequence oligonucleotides. In other implementations, a plurality of barcodes may be used such that barcodes are not necessarily unique to one another in the plurality. In these implementations, barcodes are generally attached (e.g., by ligation or PCR amplification) to individual molecules such that the combination of the barcode and the sequence it may be attached to creates a unique sequence that may be individually tracked. As described herein, detection of non-uniquely tagged barcodes in combination with sequence data of beginning (start) and end (stop) portions of sequence reads typically allows for the assignment of a unique identity to a particular molecule. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

### Nucleic Acid Amplification

Sample nucleic acids flanked by adapters are typically amplified by PCR and other amplification methods using nucleic acid primers binding to primer binding sites in adapters flanking a DNA molecule to be amplified. In some implementations, amplification methods involve cycles of extension, denaturation and annealing resulting from thermocycling, or can be isothermal as, for example, in transcription mediated amplification. Other example amplification methods that are optionally utilized, include the ligase chain reaction, strand displacement amplification, nucleic acid sequence-based amplification, and self-sustained sequence-based replication, among other approaches.

One or more rounds of amplification cycles are generally applied to introduce sample indexes/tags to a nucleic acid molecule using conventional nucleic acid amplification methods. The amplifications are typically conducted in one or more reaction mixtures. In some implementations, molecular tags and sample indexes/tags are introduced prior to and/or after sequence capturing steps are performed. In some implementations, only the molecular tags are introduced prior to probe capturing and the sample indexes/tags are introduced after sequence capturing steps are performed. In certain implementations, both the molecular tags and the sample indexes/tags are introduced prior to performing probe-based capturing steps. In some implementations, the sample indexes/tags are introduced after sequence capturing steps (i.e., enrichment of nucleic acids) are performed. Typically, sequence capturing protocols involve introducing a single-stranded nucleic acid molecule complementary to a targeted nucleic acid sequence, e.g., a coding sequence of a genomic region and mutation of such region associated with a cancer type. Typically, the amplification reactions generate a plurality of non-uniquely or uniquely tagged nucleic acid amplicons with molecular tags and sample indexes/tags at size ranging from about 200 nucleotides (nt) to about 700 nt, from 250 nt to about 350 nt, or from about 320 nt to about 550 nt. In some implementations, the amplicons have a size of about 300 nt. In some implementations, the amplicons have a size of about 500 nt.

### Nucleic Acid Enrichment

In some implementations, sequences are enriched prior to sequencing the nucleic acids. Enrichment is optionally performed for specific target regions or nonspecifically (“target sequences”). In some implementations, targeted regions of interest may be enriched with nucleic acid capture probes (“baits”) selected for one or more bait set panels using a differential tiling and capture scheme. A differential tiling and capture scheme generally uses bait sets of different relative concentrations to differentially tile (e.g., at different “resolutions”) across genomic sections associated with the baits, subject to a set of constraints (e.g., sequencer constraints such as sequencing load, utility of each bait, etc.), and capture the targeted nucleic acids at a desired level for downstream sequencing. These targeted genomic sections of interest optionally include natural or synthetic nucleotide sequences of the nucleic acid construct. In some implementations, biotin-labeled beads with probes to one or more sections of interest can be used to capture target sequences, and optionally followed by amplification of those sections, to enrich for the regions of interest.

Sequence capture typically involves the use of oligonucleotide probes that hybridize to the target nucleic acid sequence. In certain implementations, a probe set strategy involves tiling the probes across a section of interest. Such probes can be, for example, from about 60 to about 120 nucleotides in length. The set can have a depth of about 2×, 3×, 4×, 5×, 6×, 8×, 9×, 10×, 15×, 20×, 50× or more. The effectiveness of sequence capture generally depends, in part, on the length of the sequence in the target molecule that is complementary (or nearly complementary) to the sequence of the probe.

### Nucleic Acid Sequencing

After extraction and isolation of cfDNA from samples, the cfDNA may be sequenced at steps 103 and 104. Sample nucleic acids, optionally flanked by adapters, with or without prior amplification are generally subject to sequencing. Sequencing methods or commercially available formats that are optionally utilized include, for example, Sanger sequencing, high-throughput sequencing, bisulfite sequencing, pyrosequencing, sequencing-by-synthesis, single-molecule sequencing, nanopore-based sequencing, semiconductor sequencing, sequencing-by-ligation, sequencing-by-hybridization, RNA-Seq (Illumina), Digital Gene Expression (Helicos), next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, primer walking, sequencing using PacBio, SOLID, Ion Torrent, or nanopore platforms. Sequencing reactions can be performed in a variety of sample processing units, which may include multiple lanes, multiple channels, multiple wells, or other means of processing multiple sample sets substantially simultaneously. Sample processing units can also include multiple sample chambers to enable the processing of multiple runs simultaneously.

The sequencing reactions can be performed on one more nucleic acid fragment types or sections known to contain markers of cancer or of other diseases. The sequencing reactions can also be performed on any nucleic acid fragment present in the sample. The sequence reactions may provide for sequence coverage of the genome of at least about 5%, 10%, 15%, 20%, 25%, 30%, 40%, 50%, 60%, 70%, 80%, 90%, 95%, 99%, 99.9% or 100% of the genome. In other cases, sequence coverage of the genome may be less than about 5%, 10%, 15%, 20%, 25%, 30%, 40%, 50%, 60%, 70%, 80%, 90%, 95%, 99%, 99.9% or 100% of the genome.

Simultaneous sequencing reactions may be performed using multiplex sequencing techniques. In some implementations, cell-free polynucleotides are sequenced with at least about 1000, 2000, 3000, 4000, 5000, 6000, 7000, 8000, 9000, 10000, 50000, or 100,000 sequencing reactions. In other implementations, cell-free polynucleotides are sequenced with less than about 1000, 2000, 3000, 4000, 5000, 6000, 7000, 8000, 9000, 10000, 50000, or 100,000 sequencing reactions. Sequencing reactions are typically performed sequentially or simultaneously. Subsequent data analysis is generally performed on all or part of the sequencing reactions. In some implementations, data analysis is performed on at least about 1000, 2000, 3000, 4000, 5000, 6000, 7000, 8000, 9000, 10000, 50000, or 100,000 sequencing reactions. In other implementations, data analysis may be performed on less than about 1000, 2000, 3000, 4000, 5000, 6000, 7000, 8000, 9000, 10000, 50000, or 100,000 sequencing reactions. An example read depth is from about 1000 to about 50000 reads per locus (base position).

In some implementations, a nucleic acid population is prepared for sequencing by enzymatically forming blunt-ends on double-stranded nucleic acids with single-stranded overhangs at one or both ends. In these implementations, the population is typically treated with an enzyme having a 5′-3′ DNA polymerase activity and a 3′-5′ exonuclease activity in the presence of the nucleotides (e.g., A, C, G and T or U). Example enzymes or catalytic fragments thereof that are optionally used include Klenow large fragment and T4 polymerase. At 5′ overhangs, the enzyme typically extends the recessed 3′ end on the opposing strand until it is flush with the 5′ end to produce a blunt end. At 3′ overhangs, the enzyme generally digests from the 3′ end up to and sometimes beyond the 5′ end of the opposing strand. If this digestion proceeds beyond the 5′ end of the opposing strand, the gap can be filled in by an enzyme having the same polymerase activity that is used for 5′ overhangs. The formation of blunt-ends on double-stranded nucleic acids facilitates, for example, the attachment of adapters and subsequent amplification.

In some implementations, nucleic acid populations are subject to additional processing, such as the conversion of single-stranded nucleic acids to double-stranded and/or conversion of RNA to DNA. These forms of nucleic acid are also optionally linked to adapters and amplified.

With or without prior amplification, nucleic acids subject to the process of forming blunt-ends described above, and optionally other nucleic acids in a sample, can be sequenced to produce sequenced nucleic acids. A sequenced nucleic acid can refer either to the sequence of a nucleic acid (i.e., sequence information) or a nucleic acid whose sequence has been determined. Sequencing can be performed so as to provide sequence data of individual nucleic acid molecules in a sample either directly or indirectly from a consensus sequence of amplification products of an individual nucleic acid molecule in the sample.

In some implementations, double-stranded nucleic acids with single-stranded overhangs in a sample after blunt-end formation are linked at both ends to adapters including barcodes, and the sequencing determines nucleic acid sequences as well as in-line barcodes introduced by the adapters. The blunt-end DNA molecules are optionally ligated to a blunt end of an at least partially double-stranded adapter (e.g., a Y shaped or bell-shaped adapter). Alternatively, blunt ends of sample nucleic acids and adapters can be tailed with complementary nucleotides to facilitate ligation (e.g., sticky end ligation).

The nucleic acid sample is typically contacted with a sufficient number of adapters such that there is a low probability (e.g., <1 or 0.1%) that any two copies of the same nucleic acid receive the same combination of adapter barcodes from the adapters linked at both ends. The use of adapters in this manner permits identification of families of nucleic acid sequences with the same start and stop points on a reference nucleic acid and linked to the same combination of barcodes. Such a family represents sequences of amplification products of a template/parent nucleic acid in the sample before amplification. The sequences of family members can be compiled to derive consensus nucleotide(s) or a complete consensus sequence for a nucleic acid molecule in the original sample, as modified by blunt end formation and adapter attachment. In other words, the nucleotide occupying a specified position of a nucleic acid in the sample is determined to be the consensus of nucleotides occupying that corresponding position in family member sequences. Families can include sequences of one or both strands of a double-stranded nucleic acid. If members of a family include sequences of both strands from a double-stranded nucleic acid, sequences of one strand are converted to their complement for purposes of compiling all sequences to derive consensus nucleotide(s) or sequences. Some families include only a single member sequence. In this case, this sequence can be taken as the sequence of a nucleic acid in the sample before amplification. Alternatively, families with only a single member sequence may be eliminated from subsequent analysis.

Nucleotide variations in sequenced nucleic acids can be determined by comparing sequenced nucleic acids with a reference sequence. The reference sequence is often a known sequence, e.g., a known whole or partial genome sequence from a subject (e.g., a whole genome sequence of a human subject). The reference sequence can be, for example, hG19 or hG38. The sequenced nucleic acids can represent sequences determined directly for a nucleic acid in a sample, or a consensus of sequences of amplification products of such a nucleic acid, as described above. A comparison can be performed at one or more designated positions on a reference sequence. A subset of sequenced nucleic acids can be identified including a position corresponding with a designated position of the reference sequence when the respective sequences are maximally aligned. Within such a subset it can be determined which, if any, sequenced nucleic acids include a nucleotide variation at the designated position, the length of a given cfDNA fragment based upon where its endpoints (i.e., it 5′ and 3′ terminal nucleotides) map to the reference sequence, the offset of a midpoint of a given cfDNA fragment from a midpoint of a genomic region in the cfDNA fragment, and optionally which if any, include a reference nucleotide (i.e., same as in the reference sequence). If the number of sequenced nucleic acids in the subset including a nucleotide variant exceeding a selected threshold, then a variant nucleotide can be called at the designated position. The threshold can be a simple number, such as at least 1, 2, 3, 4, 5, 6, 7, 9, or 10 sequenced nucleic acids within the subset including the nucleotide variant or it can be a ratio, such as a least 0.5, 1, 2, 3, 4, 5, 10, 15, or 20 of sequenced nucleic acids within the subset that include the nucleotide variant, among other possibilities. The comparison can be repeated for any designated position of interest in the reference sequence. Sometimes a comparison can be performed for designated positions occupying at least about 20, 100, 200, or 300 contiguous positions on a reference sequence, e.g., about 20-500, or about 50-300 contiguous positions.

Additional details regarding nucleic acid sequencing, including the formats and applications described herein are also provided in, for example, Levy et al., Annual Review of Genomics and Human Genetics, 17:95-115 (2016), Liu et al., J. of Biomedicine and Biotechnology, Volume 2012, Article ID 251364:1-11 (2012), Voelkerding et al., Clinical Chem., 55:641-658 (2009), MacLean et al., Nature Rev. Microbiol., 7:287-296 (2009), Astier et al., J Am Chem Soc., 128 (5): 1705-10 (2006), U.S. Pat. Nos. 6,210,891, 6,258,568, 6,833,246, 7,115,400, 6,969,488, 5,912,148, 6,130,073, 7,169,560, 7,282,337, 7,482,120, 7,501,245, 6,818,395, 6,911,345, 7,501,245, 7,329,492, 7,170,050, 7,302,146, 7,313,308, and 7,476,503, which are each incorporated by reference in their entirety.

### Sequencing Panel

To improve the likelihood of detecting genomic regions of interest and optionally, tumor indicating mutations, the sections of DNA sequenced may comprise a panel of genes or genomic sections that comprise known genomic regions. Selection of a limited section for sequencing (e.g., a limited panel) can reduce the total sequencing needed (e.g., a total amount of nucleotides sequenced). A sequencing panel can target a plurality of different genes or regions, for example, to detect a single cancer, a set of cancers, or all cancers. Alternatively, DNA may be sequenced by whole genome sequencing (WGS) or other unbiased sequencing method without the use of a sequencing panel. Examples of suitable panel and targets for use in panels can be found in the epigenetic targets described in U.S. provisional patent application 62/799,637, filed Jan. 31, 2019, which is incorporated by reference in its entirety.

In some aspects, a panel that targets a plurality of different genes or genomic regions (e.g., transcriptional factor binding regions, distal regulatory elements (DREs), repetitive elements, intron-exon junctions, transcriptional start sites (TSSs), and/or the like) is selected such that a determined proportion of subjects having a cancer exhibits a genetic variant or tumor marker in one or more different genes in the panel. The panel may be selected to limit a region for sequencing to a fixed number of base pairs. The panel may be selected to sequence a desired amount of DNA. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for detecting one or more genetic variants in a sample.

Probes for detecting the panel of regions can include those for detecting genomic regions of interest (hotspot regions) as well as nucleosome-aware probes (e.g., KRAS codons 12 and 13) and may be designed to optimize capture based on analysis of cfDNA coverage and fragment size variation impacted by nucleosome binding patterns and GC sequence composition. Regions used herein can also include non-hotspot regions optimized based on nucleosome positions and GC models. The panel can comprise a plurality of subpanels, including subpanels for identifying tissue of origin (e.g., use of published literature to define 50-100 baits representing genes with most diverse transcription profile across tissues (not necessarily promoters)), whole genome scaffold (e.g., for identifying ultra-conservative genomic content and tiling sparsely across chromosomes with handful of probes for copy number base lining purposes), transcription start site (TSS)/CpG islands (e.g., for capturing differential methylated regions (e.g., Differentially Methylated Regions (DMRs)) in for example in promoters of tumor suppressor genes (e.g., SEPT9/VIM in colorectal cancer)). In some implementations, markers for a tissue of origin are tissue-specific epigenetic markers.

Some examples of listings of genomic locations of interest may be found in Table 1 and Table 2. In some implementations, genomic locations used in the methods of the present disclosure comprise at least a portion of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or 97 of the genes of Table 1. In some implementations, genomic locations used in the methods of the present disclosure comprise at least 5, at least 10, at least 15, at least 20, at least 25, at least 30, at least 35, at least 40, at least 45, at least 50, at least 55, at least 60, at least 65, or 70 of the SNVs of Table 1. In some implementations, genomic locations used in the methods of the present disclosure comprise at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the CNVs of Table 1. In some implementations, genomic locations used in the methods of the present disclosure comprise at least 1, at least 2, at least 3, at least 4, at least 5, or 6 of the fusions of Table 1. In some implementations, genomic locations used in the methods of the present disclosure comprise at least a portion of at least 1, at least 2, or 3 of the indels of Table 1. In some implementations, genomic locations used in the methods of the present disclosure comprise at least a portion of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, or 115 of the genes of Table 2. In some implementations, genomic locations used in the methods of the present disclosure comprise at least 5, at least 10, at least 15, at least 20, at least 25, at least 30, at least 35, at least 40, at least 45, at least 50, at least 55, at least 60, at least 65, at least 70, or 73 of the SNVs of Table 2. In some implementations, genomic locations used in the methods of the present disclosure comprise at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the CNVs of Table 2. In some implementations, genomic locations used in the methods of the present disclosure comprise at least 1, at least 2, at least 3, at least 4, at least 5, or 6 of the fusions of Table 2. In some implementations, genomic locations used in the methods of the present disclosure comprise at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 2. Each of these genomic locations of interest may be identified as a backbone region or hot-spot region for a given bait set panel. In one or more examples, the methods of the present disclosure may be implemented using all of the mutations included in Table 1 and/or Table 2.

TABLE 1

| Point Mutations (SNVs) | | | | | | Amplifications (CNVs) | | Fusions | Indels |
|---|---|---|---|---|---|---|---|---|---|
| AKT1 | ALK | APC | AR | ARAF | ARID1A | AR | BRAF | ALK | EGFR |
| ATM | BRAF | BRCA1 | BRCA2 | CCND1 | CCND2 | CCND1 | CCND2 | FGFR2 | (exons |
| CCNE1 | CDH1 | CDK4 | CDK6 | CDKN2A | CDKN2B | CCNE1 | CDK4 | FGFR3 | 19 & 20) |
| CTNNB1 | EGFR | ERBB2 | ESR1 | EZH2 | FBXW7 | CDK6 | EGFR | NTRK1 | ERBB2 |
| FGFR1 | FGFR2 | FGFR3 | GATA3 | GNA11 | GNAQ | ERBB2 | FGFR1 | RET | (exons |
| GNAS | HNF1A | HRAS | IDH1 | IDH2 | JAK2 | FGFR2 | KIT | ROS1 | 19 & 20) |
| JAK3 | KIT | KRAS | MAP2K1 | MAP2K2 | MET | KRAS | MET | | MET |
| MLH1 | MPL | MYC | NF1 | NFE2L2 | NOTCH1 | MYC | PDGFRA | | (exon 14 |
| NPM1 | NRAS | NTRK1 | PDGFRA | PIK3CA | PTEN | PIK3CA | RAF1 | | skipping) |
| PTPN11 | RAF1 | RB1 | RET | RHEB | RHOA | | | | |
| RIT1 | ROS1 | SMAD4 | SMO | SRC | STK11 | | | | |
| TERT | TP53 | TSC1 | VHL | | | | | | |

TABLE 2

| Point Mutations (SNVs) | | | | | | Amplifications (CNVs) | | Fusions | Indels |
|---|---|---|---|---|---|---|---|---|---|
| AKT1 | ALK | APC | AR | ARAF | ARID1A | AR | BRAF | ALK | EGFR |
| ATM | BRAF | BRCA1 | BRCA2 | CCND1 | CCND2 | CCND1 | CCND2 | FGFR2 | (exons |
| CCNE1 | CDH1 | CDK4 | CDK6 | CDKN2A | DDR2 | CCNE1 | CDK4 | FGFR3 | 19 & 20) |
| CTNNB1 | EGFR | ERBB2 | ESR1 | EZH2 | FBXW7 | CDK6 | EGFR | NTRK1 | ERBB2 |
| FGFR1 | FGFR2 | FGFR3 | GATA3 | GNA11 | GNAQ | ERBB2 | FGFR1 | RET | (exons |
| GNAS | HNF1A | HRAS | IDH1 | IDH2 | JAK2 | FGFR2 | KIT | ROS1 | 19 & 20) |
| JAK3 | KIT | KRAS | MAP2K1 | MAP2K2 | MET | KRAS | MET | | MET |
| MLH1 | MPL | MYC | NF1 | NFE2L2 | NOTCH1 | MYC | PDGFRA | | (exon 14 |
| NPM1 | NRAS | NTRK1 | PDGFRA | PIK3CA | PTEN | PIK3CA | RAF1 | | skipping) |
| PTPN11 | RAF1 | RB1 | RET | RHEB | RHOA | | | | ATM |
| RIT1 | ROS1 | SMAD4 | SMO | MAPK1 | STK11 | | | | APC |
| TERT | TP53 | TSC1 | VHL | MAPK3 | MTOR | | | | ARID1A |
| NTRK3 | | | | | | | | | BRCA1 |
| | | | | | | | | | BRCA2 |
| | | | | | | | | | CDH1 |
| | | | | | | | | | CDKN2A |
| | | | | | | | | | GATA3 |
| | | | | | | | | | KIT |
| | | | | | | | | | MLH1 |
| | | | | | | | | | MTOR |
| | | | | | | | | | NF1 |
| | | | | | | | | | PDGFRA |
| | | | | | | | | | PTEN |
| | | | | | | | | | RB1 |
| | | | | | | | | | SMAD4 |
| | | | | | | | | | STK11 |
| | | | | | | | | | TP53 |
| | | | | | | | | | TSC1 |
| | | | | | | | | | VHL |

In some implementations, the one or more regions in the panel comprise one or more loci from one or a plurality of genes for detecting residual cancer after surgery. This detection can be earlier than is possible for existing methods of cancer detection. In some implementations, the one or more genomic locations in the panel comprise one or more loci from one or a plurality of genes for detecting cancer in a high-risk patient population. For example, smokers have much higher rates of lung cancer than the general population. Moreover, smokers can develop other lung conditions that make cancer detection more difficult, such as the development of irregular nodules in the lungs. In some implementations, the methods described herein detect the response of patients to cancer therapy (particularly in high risk patients) earlier than is possible for existing methods of cancer detection.

A genomic location may be selected for inclusion in a sequencing panel based on a number of subjects with a cancer that have a tumor marker in that gene or region. A genomic location may be selected for inclusion in a sequencing panel based on prevalence of subjects with a cancer and a tumor marker present in that gene. Presence of a tumor marker in a region may be indicative of a subject having cancer.

In some instances, the panel may be selected using information from one or more databases. The information regarding a cancer may be derived from cancer tumor biopsies or cfDNA assays. A database may comprise information describing a population of sequenced tumor samples. A database may comprise information about mRNA expression in tumor samples. A database may comprise information about regulatory elements or genomic regions in tumor samples. The information relating to the sequenced tumor samples may include the frequency of various genetic variants and describe the genes or regions in which the genetic variants occur. The genetic variants may be tumor markers. A non-limiting example of such a database is COSMIC. COSMIC is a catalogue of somatic mutations found in various cancers. For a particular cancer, COSMIC ranks genes based on frequency of mutation. A gene may be selected for inclusion in a panel by having a high frequency of mutation within a given gene. For instance, COSMIC indicates that 33% of a population of sequenced breast cancer samples have a mutation in TP53 and 22% of a population of sampled breast cancers have a mutation in KRAS. Other ranked genes, including APC, have mutations found only in about 4% of a population of sequenced breast cancer samples. TP53 and KRAS may be included in a sequencing panel based on having relatively high frequency among sampled breast cancers (compared to APC, for example, which occurs at a frequency of about 4%). COSMIC is provided as a non-limiting example, however, any database or set of information may be used that associates a cancer with tumor marker located in a gene or genetic region. In another example, as provided by COSMIC, of 1156 biliary tract cancer samples, 380 samples (33%) carried mutations in TP53. Several other genes, such as APC, have mutations in 4-8% of all samples. Thus, TP53 may be selected for inclusion in the panel based on a relatively high frequency in a population of biliary tract cancer samples.

A gene or genomic section may be selected for a panel where the frequency of a tumor marker is significantly greater in sampled tumor tissue or circulating tumor DNA than found in a given background population. A combination of genomic locations may be selected for inclusion of a panel such that at least a majority of subjects having a cancer may have a tumor marker or genomic region present in at least one of the genomic location or genes in the panel. The combination of genomic location may be selected based on data indicating that, for a particular cancer or set of cancers, a majority of subjects have one or more tumor markers in one or more of the selected regions. For example, to detect cancer 1, a panel comprising regions A, B, C, and/or D may be selected based on data indicating that 90% of subjects with cancer 1 have a tumor marker in regions A, B, C, and/or D of the panel. Alternately, tumor markers may be shown to occur independently in two or more regions in subjects having a cancer such that, combined, a tumor marker in the two or more regions is present in a majority of a population of subjects having a cancer. For example, to detect cancer 2, a panel comprising regions X, Y, and Z may be selected based on data indicating that 90% of subjects have a tumor marker in one or more regions, and in 30% of such subjects a tumor marker is detected only in region X, while tumor markers are detected only in regions Y and/or Z for the remainder of the subjects for whom a tumor marker was detected. Tumor markers present in one or more genomic locations previously shown to be associated with one or more cancers may be indicative of or predictive of a subject having cancer if a tumor marker is detected in one or more of those regions 50% or more of the time. Computational approaches such as models employing conditional probabilities of detecting cancer given a cancer frequency for a set of tumor markers within one or more regions may be used to predict which regions, alone or in combination, may be predictive of cancer. Other approaches for panel selection involve the use of databases describing information from studies employing comprehensive genomic profiling of tumors with large panels and/or whole genome sequencing (WGS, RNA-seq, Chip-seq, ATAC-seq, and others). Information gleaned from literature may also describe pathways commonly affected and mutated in certain cancers. Panel selection may be further informed by the use of ontologies describing genetic information.

Genes included in the panel for sequencing can include the fully transcribed region, the promoter region, enhancer regions, regulatory elements, and/or downstream sequence. To further increase the likelihood of detecting tumor indicating mutations only exons may be included in the panel. The panel can comprise all exons of a selected gene, or only one or more of the exons of a selected gene. The panel may comprise of exons from each of a plurality of different genes. The panel may comprise at least one exon from each of the plurality of different genes.

In some aspects, a panel of exons from each of a plurality of different genes is selected such that a determined proportion of subjects having a cancer exhibit a genetic variant in at least one exon in the panel of exons.

At least one full exon from each different gene in a panel of genes may be sequenced. The sequenced panel may comprise exons from a plurality of genes. The panel may comprise exons from 2 to 100 different genes, from 2 to 70 genes, from 2 to 50 genes, from 2 to 30 genes, from 2 to 15 genes, or from 2 to 10 genes.

A selected panel may comprise a varying number of exons. The panel may comprise from 2 to 3000 exons. The panel may comprise from 2 to 1000 exons. The panel may comprise from 2 to 500 exons. The panel may comprise from 2 to 100 exons. The panel may comprise from 2 to 50 exons. The panel may comprise no more than 300 exons. The panel may comprise no more than 200 exons. The panel may comprise no more than 100 exons. The panel may comprise no more than 50 exons. The panel may comprise no more than 40 exons. The panel may comprise no more than 30 exons. The panel may comprise no more than 25 exons. The panel may comprise no more than 20 exons. The panel may comprise no more than 15 exons. The panel may comprise no more than 10 exons. The panel may comprise no more than 9 exons. The panel may comprise no more than 8 exons. The panel may comprise no more than 7 exons.

The panel may comprise one or more exons from a plurality of different genes. The panel may comprise one or more exons from each of a proportion of the plurality of different genes. The panel may comprise at least two exons from each of at least 25%, 50%, 75% or 90% of the different genes. The panel may comprise at least three exons from each of at least 25%, 50%, 75% or 90% of the different genes. The panel may comprise at least four exons from each of at least 25%, 50%, 75% or 90% of the different genes.

The sizes of the sequencing panel may vary. A sequencing panel may be made larger or smaller (in terms of nucleotide size) depending on several factors including, for example, the total amount of nucleotides sequenced, or a number of unique molecules sequenced for a particular region in the panel. The sequencing panel can be sized 5 kb to 50 kb. The sequencing panel can be 10 kb to 30 kb in size. The sequencing panel can be 12 kb to 20 kb in size. The sequencing panel can be 12 kb to 60 kb in size. The sequencing panel can be at least 10 kb, 12 kb, 15 kb, 20 kb, 25 kb, 30 kb, 35 kb, 40 kb, 45 kb, 50 kb, 60 kb, 70 kb, 80 kb, 90 kb, 100 kb, 110 kb, 120 kb, 130 kb, 140 kb, or 150 kb in size. The sequencing panel may be less than 100 kb, 90 kb, 80 kb, 70 kb, 60 kb, or 50 kb in size.

The panel selected for sequencing can comprise at least 1, 5, 10, 15, 20, 25, 30, 40, 50, 60, 80, or 100 genomic locations (e.g., that each include genomic regions of interest). In some cases, the genomic locations in the panel are selected that the size of the locations are relatively small. In some cases, the regions in the panel have a size of about 10 kb or less, about 8 kb or less, about 6 kb or less, about 5 kb or less, about 4 kb or less, about 3 kb or less, about 2.5 kb or less, about 2 kb or less, about 1.5 kb or less, or about 1 kb or less or less. In some cases, the genomic locations in the panel have a size from about 0.5 kb to about 10 kb, from about 0.5 kb to about 6 kb, from about 1 kb to about 11 kb, from about 1 kb to about 15 kb, from about 1 kb to about 20 kb, from about 0.1 kb to about 10 kb, or from about 0.2 kb to about 1 kb. For example, the regions in the panel can have a size from about 0.1 kb to about 5 kb.

The panel selected herein can allow for deep sequencing that is sufficient to detect low-frequency genetic variants (e.g., in cell-free nucleic acid molecules obtained from a sample). An amount of genetic variants in a sample may be referred to in terms of the mutant allele fraction for a given genetic variant. The mutant allele fraction may refer to the frequency at which mutant alleles occur in a given population of nucleic acids, such as a sample. Genetic variants at a low mutant allele fraction may have a relatively low frequency of presence in a sample. In some cases, the panel allows for detection of genetic variants at a mutant allele fraction of at least 0.0001%, 0.001%, 0.005%, 0.01%, 0.05%, 0.1%, or 0.5%. The panel can allow for detection of genetic variants at a mutant allele fraction of 0.001% or greater. The panel can allow for detection of genetic variants at a mutant allele fraction of 0.01% or greater. The panel can allow for detection of genetic variant present in a sample at a frequency of as low as 0.0001%, 0.001%, 0.005%, 0.01%, 0.025%, 0.05%, 0.075%, 0.1%, 0.25%, 0.5%, 0.75%, or 1.0%. The panel can allow for detection of tumor markers present in a sample at a frequency of at least 0.0001%, 0.001%, 0.005%, 0.01%, 0.025%, 0.05%, 0.075%, 0.1%, 0.25%, 0.5%, 0.75%, or 1.0%. The panel can allow for detection of tumor markers at a frequency in a sample as low as 1.0%. The panel can allow for detection of tumor markers at a frequency in a sample as low as 0.75%. The panel can allow for detection of tumor markers at a frequency in a sample as low as 0.5%. The panel can allow for detection of tumor markers at a frequency in a sample as low as 0.25%. The panel can allow for detection of tumor markers at a frequency in a sample as low as 0.1%. The panel can allow for detection of tumor markers at a frequency in a sample as low as 0.075%. The panel can allow for detection of tumor markers at a frequency in a sample as low as 0.05%. The panel can allow for detection of tumor markers at a frequency in a sample as low as 0.025%. The panel can allow for detection of tumor markers at a frequency in a sample as low as 0.01%. The panel can allow for detection of tumor markers at a frequency in a sample as low as 0.005%. The panel can allow for detection of tumor markers at a frequency in a sample as low as 0.001%. The panel can allow for detection of tumor markers at a frequency in a sample as low as 0.0001%. The panel can allow for detection of tumor markers in sequenced cfDNA at a frequency in a sample as low as 1.0% to 0.0001%. The panel can allow for detection of tumor markers in sequenced cfDNA at a frequency in a sample as low as 0.01% to 0.0001%.

A genetic variant can be exhibited in a percentage of a population of subjects who have a disease (e.g., cancer). In some cases, at least 1%, 2%, 3%, 5%, 10%, 20%, 30%, 40%, 50%, 60%, 70%, 80%, 90%, 95%, or 99% of a population having the cancer exhibit one or more genetic variants in at least one of the regions in the panel. For example, at least 80% of a population having the cancer may exhibit one or more genetic variants in at least one of the genomic positions in the panel.

The panel can comprise one or more locations comprising genomic regions of interest from each of one or more genes. In some cases, the panel can comprise one or more locations comprising genomic regions of interest from each of at least 1, 2, 3, 4, 5, 6, 7, 8, 9, 10, 15, 20, 25, 30, 40, 50, or 80 genes. In some cases, the panel can comprise one or more locations comprising genomic regions of interest from each of at most 1, 2, 3, 4, 5, 6, 7, 8, 9, 10, 15, 20, 25, 30, 40, 50, or 80 genes. In some cases, the panel can comprise one or more locations comprising genomic regions of interest from each of from about 1 to about 80, from 1 to about 50, from about 3 to about 40, from 5 to about 30, from 10 to about 20 different genes.

The locations comprising genomic regions in the panel can be selected so that one or more epigenetically modified regions are detected. The one or more epigenetically modified regions can be acetylated, methylated, ubiquitylated, phosphorylated, sumoylated, ribosylated, and/or citrullinated. For example, the regions in the panel can be selected so that one or more methylated regions are detected.

The regions in the panel can be selected so that they comprise sequences differentially transcribed across one or more tissues. In some cases, the locations comprising genomic regions can comprise sequences transcribed in certain tissues at a higher level compared to other tissues. For example, the locations comprising genomic regions can comprise sequences transcribed in certain tissues but not in other tissues.

The genomic locations in the panel can comprise coding and/or non-coding sequences. For example, the genomic locations in the panel can comprise one or more sequences in exons, introns, promoters, 3′ untranslated regions, 5′ untranslated regions, regulatory elements, transcription start sites, and/or splice sites. In some cases, the regions in the panel can comprise other non-coding sequences, including pseudogenes, repeat sequences, transposons, viral elements, and telomeres. In some cases, the genomic locations in the panel can comprise sequences in non-coding RNA, e.g., ribosomal RNA, transfer RNA, Piwi-interacting RNA, and microRNA.

The genomic locations in the panel can be selected to detect (diagnose) a cancer with a desired level of sensitivity (e.g., through the detection of one or more genetic variants). For example, the regions in the panel can be selected to detect the cancer (e.g., through the detection of one or more genetic variants) with a sensitivity of at least 50%, 55%, 60%, 65%, 70%, 75%, 80%, 85%, 90%, 95%, 96%, 97%, 98%, 99%, 99.5%, or 99.9%. The genomic locations in the panel can be selected to detect the cancer with a sensitivity of 100%.

The genomic locations in the panel can be selected to detect (diagnose) a cancer with a desired level of specificity (e.g., through the detection of one or more genetic variants). For example, the genomic locations in the panel can be selected to detect cancer (e.g., through the detection of one or more genetic variants) with a specificity of at least 50%, 55%, 60%, 65%, 70%, 75%, 80%, 85%, 90%, 95%, 96%, 97%, 98%, 99%, 99.5%, or 99.9%. The genomic locations in the panel can be selected to detect the one or more genetic variant with a specificity of 100%.

The genomic locations in the panel can be selected to detect (diagnose) a cancer with a desired positive predictive value. Positive predictive value can be increased by increasing sensitivity (e.g., chance of an actual positive being detected) and/or specificity (e.g., chance of not mistaking an actual negative for a positive). As a non-limiting example, genomic locations in the panel can be selected to detect the one or more genetic variant with a positive predictive value of at least 50%, 55%, 60%, 65%, 70%, 75%, 80%, 85%, 90%, 95%, 96%, 97%, 98%, 99%, 99.5%, or 99.9%. The regions in the panel can be selected to detect the one or more genetic variant with a positive predictive value of 100%.

The genomic locations in the panel can be selected to detect (diagnose) a cancer with a desired accuracy. As used herein, the term “accuracy” may refer to the ability of a test to discriminate between a disease condition (e.g., cancer) and healthy condition. Accuracy can be quantified using measures such as sensitivity and specificity, predictive values, likelihood ratios, the area under the ROC curve, Youden's index and/or diagnostic odds ratio.

Accuracy may be presented as a percentage, which refers to a ratio between the number of tests giving a correct result and the total number of tests performed. The regions in the panel can be selected to detect cancer with an accuracy of at least 50%, 55%, 60%, 65%, 70%, 75%, 80%, 85%, 90%, 95%, 96%, 97%, 98%, 99%, 99.5%, or 99.9%. The genomic locations in the panel can be selected to detect cancer with an accuracy of 100%.

A panel may be selected to be highly sensitive and detect low frequency genetic variants. For instance, a panel may be selected such that a genetic variant or tumor marker present in a sample at a frequency as low as 0.01%, 0.05%, or 0.001% may be detected at a sensitivity of at least 50%, 55%, 60%, 65%, 70%, 75%, 80%, 85%, 90%, 95%, 96%, 97%, 98%, 99%, 99.5%, or 99.9%. Genomic locations in a panel may be selected to detect a tumor marker present at a frequency of 1% or less in a sample with a sensitivity of 70% or greater. A panel may be selected to detect a tumor marker at a frequency in a sample as low as 0.1% with a sensitivity of at least 50%, 55%, 60%, 65%, 70%, 75%, 80%, 85%, 90%, 95%, 96%, 97%, 98%, 99%, 99.5%, or 99.9%. A panel may be selected to detect a tumor marker at a frequency in a sample as low as 0.01% with a sensitivity of at least 50%, 55%, 60%, 65%, 70%, 75%, 80%, 85%, 90%, 95%, 96%, 97%, 98%, 99%, 99.5%, or 99.9%. A panel may be selected to detect a tumor marker at a frequency in a sample as low as 0.001% with a sensitivity of at least 50%, 55%, 60%, 65%, 70%, 75%, 80%, 85%, 90%, 95%, 96%, 97%, 98%, 99%, 99.5%, or 99.9%.

A panel may be selected to be highly specific and detect low frequency genetic variants. For instance, a panel may be selected such that a genetic variant or tumor marker present in a sample at a frequency as low as 0.01%, 0.05%, or 0.001% may be detected at a specificity of at least 50%, 55%, 60%, 65%, 70%, 75%, 80%, 85%, 90%, 95%, 96%, 97%, 98%, 99%, 99.5%, or 99.9%. Genomic locations in a panel may be selected to detect a tumor marker present at a frequency of 1% or less in a sample with a specificity of 70% or greater. A panel may be selected to detect a tumor marker at a frequency in a sample as low as 0.1% with a specificity of at least 70%, 75%, 80%, 85%, 90%, 95%, 96%, 97%, 98%, 99%, 99.5%, or 99.9%. A panel may be selected to detect a tumor marker at a frequency in a sample as low as 0.01% with a specificity of at least 70%, 75%, 80%, 85%, 90%, 95%, 96%, 97%, 98%, 99%, 99.5%, or 99.9%. A panel may be selected to detect a tumor marker at a frequency in a sample as low as 0.001% with a specificity of at least 70%, 75%, 80%, 85%, 90%, 95%, 96%, 97%, 98%, 99%, 99.5%, or 99.9%.

A panel may be selected to be highly accurate and detect low frequency genetic variants. A panel may be selected such that a genetic variant or tumor marker present in a sample at a frequency as low as 0.01%, 0.05%, or 0.001% may be detected at an accuracy of at least 70%, 75%, 80%, 85%, 90%, 95%, 96%, 97%, 98%, 99%, 99.5%, or 99.9%. Genomic locations in a panel may be selected to detect a tumor marker present at a frequency of 1% or less in a sample with an accuracy of 70% or greater. A panel may be selected to detect a tumor marker at a frequency in a sample as low as 0.1% with an accuracy of at least 70%, 75%, 80%, 85%, 90%, 95%, 96%, 97%, 98%, 99%, 99.5%, or 99.9%. A panel may be selected to detect a tumor marker at a frequency in a sample as low as 0.01% with an accuracy of at least 70%, 75%, 80%, 85%, 90%, 95%, 96%, 97%, 98%, 99%, 99.5%, or 99.9%. A panel may be selected to detect a tumor marker at a frequency in a sample as low as 0.001% with an accuracy of at least 70%, 75%, 80%, 85%, 90%, 95%, 96%, 97%, 98%, 99%, 99.5%, or 99.9%.

A panel may be selected to be highly predictive and detect low frequency genetic variants. A panel may be selected such that a genetic variant or tumor marker present in a sample at a frequency as low as 0.01%, 0.05%, or 0.001% may have a positive predictive value of at least 70%, 75%, 80%, 85%, 90%, 95%, 96%, 97%, 98%, 99%, 99.5%, or 99.9%.

The concentration of probes or baits used in the panel may be increased (2 to 6 ng/μL) to capture more nucleic acid molecule within a sample. The concentration of probes or baits used in the panel may be at least 2 ng/μL, 3 ng/μL, 4 ng/μL, 5 ng/μL, 6 ng/μL, or greater. The concentration of probes may be about 2 ng/μL to about 3 ng/μL, about 2 ng/μL to about 4 ng/μL, about 2 ng/μL to about 5 ng/μL, about 2 ng/μL to about 6 ng/μL. The concentration of probes or baits used in the panel may be 2 ng/μL or more to 6 ng/μL or less. In some instances, this may allow for more molecules within a biological to be analyzed thereby enabling lower frequency alleles to be detected.

In an implementation, after sequencing, sequence reads may be assigned a quality score. A quality score may be a representation of sequence reads that indicates whether those sequence reads may be useful in subsequent analysis based on a threshold. In some cases, some sequence reads are not of sufficient quality or length to perform a subsequent mapping step. Sequence reads with a quality score at least 90%, 95%, 99%, 99.9%, 99.99% or 99.999% may be filtered out of a data set of sequence reads. In other cases, sequence reads assigned a quality scored at least 90%, 95%, 99%, 99.9%, 99.99% or 99.999% may be filtered out of the data set. Sequence reads that meet a specified quality score threshold may be mapped to a reference genome. After mapping alignment, sequence reads may be assigned a mapping score. A mapping score may be a representation of sequence reads mapped back to the reference sequence indicating whether each position is or is not uniquely mappable. Sequence reads with a mapping score at least 90%, 95%, 99%, 99.9%, 99.99% or 99.999% may be filtered out of the data set. In other cases, sequencing reads assigned a mapping scored less than 90%, 95%, 99%, 99.9%, 99.99% or 99.999% may be filtered out of the data set.

### Precision Treatments

The precision diagnostics provided by the improved computer system 110 may result in precision treatment plans, which may be identified by the computer system 110 (and/or curated by health professionals). For example, one type of precision diagnostic and treatment may relate to genes in the homologous recombination repair (HRR) pathway.

Homologous recombination is a type of genetic recombination in which nucleotide sequences are exchanged between two similar or identical molecules of DNA. It is most widely used by cells to accurately repair harmful breaks that occur on both strands of DNA, known as double-strand breaks (DSB). HRR provides a mechanism for the error-free removal of damage present in DNA that has replicated (S and G2 phases), to eliminate chromosomal breaks before the cell division occurs. The primary model for how homologous recombination repairs double-strand breaks in DNA is homologous recombination repair pathway which mediates the double-strand break repair (DSBR) pathway and the synthesis-dependent strand annealing (SDSA) pathway. Germline and somatic deficiencies in homologous recombination genes have been strongly linked to breast, ovarian and prostate cancers.

The number and types of variant nucleotides in a sample can provide an indication of the amenability of the subject providing the sample to treatment, i.e., therapeutic intervention. For example, various poly ADP ribose polymerase (PARP) inhibitors have been shown to stop the growth of tumors from breast, ovarian and prostate cancers caused by hereditary mutations in the BRCA1 or BRCA2 genes. Some of these therapeutic agents may inhibit base excision repair (BER), which may compensate for the deficiency of HRR.

On the other hand, certain BRCA and HRR wildtype patients may not achieve clinical benefit from treatment with a PARP inhibitor. Furthermore, not all ovarian cancer patients with a BRCA mutation will respond to a PARP inhibitor. Moreover, different types of mutations may indicate different therapies. For example, somatic heterozygous deletions in HRR genes may indicate a different therapy than somatic homozygous deletions. Thus, the state of genetic material may influence therapy. In one example, a PARP inhibitor may be administered to an individual harboring a somatic homozygous deletion in a HRR gene, but not to an individual harboring a wildtype allele or somatic heterozygous deletions in the HRR gene.

In some implementations, a subject having HRD as determined by any of the methods disclosed may be administered a targeted therapy. The targeted therapy may comprise a PARP inhibitor. Examples of PARP inhibitors that may be administered include one or more of: VELIPARIB, OLAPARIB, TALAZOPARIB, RUCAPARIB, NIRAPARIB, PAMIPARIB, CEP 9722 (Cephalon), E7016 (Eisai), E7449 (Eisai, a PARP ½ and tankyrase ½ inhibitor), or 3-Aminobenzamide. In some implementations, the targeted therapy may comprise at least one base excision repair (BER) inhibitor. For example, OLAPARIB may inhibit BER. In certain implementations, the targeted therapy may comprise combination of a PARP inhibitor and radiotherapy. In an implementation, the combination of a PARP inhibitor and radiotherapy would permit the PARP inhibitor to lead to formation of double strand breaks from the single-strand breaks generated by the radiotherapy in tumor tissue (e.g., tissue with BRCA1/BRCA2 mutations). This combination can provide more powerful therapy per radiation dose.

### Customized Therapies and Related Administrations

In some implementations, the methods disclosed herein relate to identifying and administering therapies to patients having a given disease, disorder or condition. Essentially any cancer therapy (e.g., surgical therapy, radiation therapy, chemotherapy, and/or the like) is included as part of these methods. This includes, for example, the disease, disorder or condition found in Table 3.

TABLE 3
Disease and Treatments

| Cancer type | Drug | Target |
|---|---|---|
| Breast | abemaciclib | CDK4, CDK6 |
| | (Verzenio) | |
| prostate | abiraterone acetate | CYP17A1 |
| | (Zytiga) | |
| leukemia | acalabrutinib | BTK |
| | (Calquence) | |
| lymphoma | acalabrutinib | BTK |
| | (Calquence) | |
| lung | adagrasib (Krazati) | KRAS G12C |
| Breast | ado-trastuzumab | Tubulin, Her2 |
| | emtansine (Kadcyla) | |
| lung | afatinib dimaleate | EGFR + EGFR exon 21 L858R mutation + |
| | (Gilotrif) | EGFR-Ex19del + HER2 + HER4 |
| lung | alectinib (Alecensa) | ALK, RET |
| leukemia | alemtuzumab | CD52 |
| | (Campath) | |
| skin | alitretinoin (Panretin) | RARα + RARβ2 + RARγ |
| soft tissue sarcoma | alitretinoin (Panretin) | RARα + RARβ2 + RARγ |
| Breast | alpelisib (Piqray) | PI3Kα |
| lung | amivantamab-vmjw | EGFR + c-MET |
| | (Rybrevant) | |
| Breast | anastrozole | Aromatase |
| | (Arimidex) | |
| prostate | apalutamide (Erleada) | AR |
| leukemia | asciminib | Bcr-Abl |
| | hydrochloride | |
| | (Scemblix) | |
| bladder | atezolizumab | PDL1 |
| | (Tecentriq) | |
| liver and bile duct | atezolizumab | PDL1 |
| | (Tecentriq) | |
| lung | atezolizumab | PDL1 |
| | (Tecentriq) | |
| skin | atezolizumab | PDL1 |
| | (Tecentriq) | |
| soft tissue sarcoma | atezolizumab | PDL1 |
| | (Tecentriq) | |
| Gastrointestinal | avapritinib (Ayvakit) | PDGFRα + c-Kit |
| systemic | avapritinib (Ayvakit) | PDGFRα + c-Kit |
| mastocytosis | | |
| bladder | avelumab (Bavencio) | PDL1 |
| endocrine and | avelumab (Bavencio) | PDL1 |
| neuroendocrine | | |
| tumors | | |
| kidney | avelumab (Bavencio) | PDL1 |
| skin | avelumab (Bavencio) | PDL1 |
| lymphoma | axicabtagene | CD19 |
| | ciloleucel (Yescarta) | |
| kidney | axitinib (Inlyta) | VEGFR1 + VEGFR2 + VEGFR3 |
| lymphoma | belinostat (Beleodaq) | HDAC |
| Brain | belzutifan (Welireg) | HIF-2A |
| kidney | belzutifan (Welireg) | HIF-2A |
| pancreatic | belzutifan (Welireg) | HIF-2A |
| ovarian epithelial, | bevacizumab | VEGF-A |
| fallopian tube, and | (Avastin) | |
| primary peritoneal | | |
| Brain | bevacizumab | VEGF-A |
| | (Avastin) | |
| Cervical | bevacizumab | VEGF-A |
| | (Avastin) | |
| colorectal | bevacizumab | VEGF-A |
| | (Avastin) | |
| kidney | bevacizumab | VEGF-A |
| | (Avastin) | |
| liver and bile duct | bevacizumab | VEGF-A |
| | (Avastin) | |
| lung | bevacizumab | VEGF-A |
| | (Avastin) | |
| lymphoma | bexarotene | RXRs |
| | (Targretin) | |
| skin | binimetinib (Mektovi) | MEK1, MEK2 |
| leukemia | blinatumomab | CD19, CD3 |
| | (Blincyto) | |
| lymphoma | bortezomib (Velcade) | Proteasome |
| multiple myeloma | bortezomib (Velcade) | Proteasome |
| leukemia | bosutinib (Bosulif) | BCR-ABL, SRC |
| lymphoma | brentuximab vedotin | CD130, Tubulin |
| | (Adcetris) | |
| leukemia | brexucabtagene | CD19 |
| | autoleucel (Tecartus) | |
| lymphoma | brexucabtagene | CD19 |
| | autoleucel (Tecartus) | |
| lung | brigatinib (Alunbrig) | ALK, EGFR, FLT3, IGF-1R, ROS1 |
| prostate | cabazitaxel (Jevtana) | Tubulin |
| kidney | cabozantinib-s-malate | AXL + RET + ROS1 + TYRO3 + Tie-2 + TrkB + |
| | (Cabometyx) | VEGFR1 + VEGFR2 + VEGFR3 + c-Kit + c-Met |
| liver and bile duct | cabozantinib-s-malate | AXL + RET + ROS1 + TYRO3 + Tie-2 + TrkB + |
| | (Cabometyx) | VEGFR1 + VEGFR2 + VEGFR3 + c-Kit + c-Met |
| thyroid | cabozantinib-s-malate | AXL + RET + ROS1 + TYRO3 + Tie-2 + TrkB + |
| | (Cometriq) | VEGFR1 + VEGFR2 + VEGFR3 + c-Kit + c-Met |
| lung | capmatinib | c-Met |
| | hydrochloride | |
| | (Tabrecta) | |
| multiple myeloma | carfilzomib (Kyprolis) | protoasome |
| lung | cemiplimab-rwlc | PD-1 |
| | (Libtayo) | |
| skin | cemiplimab-rwlc | PD-1 |
| | (Libtayo) | |
| lung | ceritinib (Zykadia) | ALK + IGF-1R + INSR + ROS1 |
| colorectal | cetuximab (Erbitux) | EGFR |
| multiple myeloma | ciltacabtagene | BCMA |
| | autoleucel (Carvykti) | |
| skin | cobimetinib fumarate | MEK1, MEK2 |
| | (Cotellic) | |
| lymphoma | copanlisib | PI3Kα, PI3Kδ |
| | hydrochloride | |
| | (Aliqopa) | |
| lung | crizotinib (Xalkori) | MTH1 |
| lymphoma | crizotinib (Xalkori) | MTH1 |
| Myofibroblastic | crizotinib (Xalkori) | MTH1 |
| Brain | dabrafenib (Tafinlar) | BRAF, CRAF |
| lung | dabrafenib mesylate | BRAF, CRAF |
| | (Tafinlar) | |
| skin | dabrafenib mesylate | BRAF, CRAF |
| | (Tafinlar) | |
| solid tumors | dabrafenib mesylate | BRAF, CRAF |
| anywhere in the | (Tafinlar) | |
| body | | |
| thyroid | dabrafenib mesylate | BRAF, CRAF |
| | (Tafinlar) | |
| lung | dacomitinib | EGFR + EGFR exon 21 L858R mutation + |
| | (Vizimpro) | EGFR-Ex19del + HER2 + HER4 |
| multiple myeloma | daratumumab | CD38 + Hyaluronic acid |
| | (Darzalex) | |
| multiple myeloma | daratumumab and | CD38 + Hyaluronic acid |
| | hyaluronidase-fihj | |
| | (Darzalex Faspro) | |
| prostate | darolutamide | AR |
| | (Nubeqa) | |
| leukemia | dasatinib (Sprycel) | Bcr-Abl + EphA2 + FYN + LCK + PDGFRβ + |
| | | Protein-tyrosine kinases + SRC + YES1 + c-Kit |
| neuroblastoma | dinutuximab | GD2 |
| | (Unituxin) | |
| endometrial | dostarlimab-gxly | PD-1 |
| | (Jemperli) | |
| solid tumors | dostarlimab-gxly | PD-1 |
| anywhere in the | (Jemperli) | |
| body | | |
| liver and bile duct | durvalumab (Imfinzi) | PDL1 |
| lung | durvalumab (Imfinzi) | PDL1 |
| leukemia | duvelisib (Copiktra) | PI3Kγ + PI3Kδ |
| Breast | elacestrant | Erα/ESR1 |
| | dihydrochloride | |
| | (Orserdu) | |
| multiple myeloma | elotuzumab | SLAMF7 |
| | (Empliciti) | |
| leukemia | enasidenib mesylate | IDH2 |
| | (Idhifa) | |
| colorectal | encorafenib (Braftovi) | BRAF, BRAF V600E |
| skin | encorafenib (Braftovi) | BRAF V600E, V600K mutation |
| bladder | enfortumab vedotin- | Nectin-4, Tubulins |
| | ejfv (Padcev) | |
| lung | entrectinib (Rozlytrek) | ALK + ROS1 + TRKA + TrkB + TrkC |
| solid tumors | entrectinib (Rozlytrek) | ALK + ROS1 + TRKA + TrkB + TrkC |
| anywhere in the | | |
| body | | |
| prostate | enzalutamide (Xtandi) | AR |
| bladder | erdafitinib (Balversa) | FGFR1, FGFR2, FGFR3, FGFR4 |
| lung | erlotinib | EGFR antagonists, EGFR exon 21 L858R |
| | hydrochloride | mutation |
| | (Tarceva) | inhibitors, EGFR-Ex19del inhibitors |
| pancreatic | erlotinib | EGFR + EGFR exon 21 L858R mutation + EGFR- |
| | hydrochloride | Ex19del |
| | (Tarceva) | |
| Breast | everolimus (Afinitor) | mTORC1, mTORC2 |
| kidney | everolimus (Afinitor) | mTORC1, mTORC2 |
| pancreatic | everolimus (Afinitor) | mTORC1, mTORC2 |
| Breast | exemestane | Aromatase |
| | (Aromasin) | |
| Breast | fam-trastuzumab | HER2, TOP1 |
| | deruxtecan-nxki | |
| | (Enhertu) | |
| Gastric | fam-trastuzumab | HER2, TOP1 |
| | deruxtecan-nxki | |
| | (Enhertu) | |
| lung | fam-trastuzumab | HER2, TOP1 |
| | deruxtecan-nxki | |
| | (Enhertu) | |
| myelodysplastic | fedratinib | JAK2, FLT3 |
| and | hydrochloride | |
| myeloproliferative | (Inrebic) | |
| disorders | | |
| Breast | fulvestrant (Faslodex) | ER |
| liver and bile duct | futibatinib (Lytgobi) | FGFRs |
| lung | gefitinib (Iressa) | EGFR + EGFR exon 21 L858R mutation + EGFR- |
| | | Ex19del |
| leukemia | gemtuzumab | CD33, DNA |
| | ozogamicin | |
| | (MYLOTARG) | |
| leukemia | gilteritinib fumarate | AXL + FLT3 |
| | (Xospata) | |
| leukemia | glasdegib | SMO |
| | maleate (Daurismo) | |
| lymphoma | ibritumomab tiuxetan | CD20 |
| | (Zevalin) | |
| leukemia | ibrutinib (Imbruvica) | BTK |
| lymphoma | ibrutinib (Imbruvica) | BTK |
| multiple myeloma | idecabtagene | BCMA |
| | vicleucel (Abecma) | |
| leukemia | idelalisib (Zydelig) | PI3Kδ |
| dermatofibrosarcoma | imatinib mesylate | BCR-ABL, PDGFR, C-Kit |
| protuberans | (Gleevec) | |
| Gastrointestinal | imatinib mesylate | BCR-ABL, PDGFR, C-Kit |
| | (Gleevec) | |
| leukemia | imatinib mesylate | BCR-ABL, PDGFR, C-Kit |
| | (Gleevec) | |
| myelodysplastic | imatinib mesylate | BCR-ABL, PDGFR, C-Kit |
| and | (Gleevec) | |
| myeloproliferative | | |
| disorders | | |
| systemic | imatinib mesylate | BCR-ABL, PDGFR, C-Kit |
| mastocytosis | (Gleevec) | |
| liver and bile duct | infigratinib phosphate | FGFR1 + FGFR2 + FGFR3 + FGFR4 |
| | (Truseltiq) | |
| leukemia | inotuzumab | CD22 + DNA |
| | ozogamicin | |
| | (Besponsa) | |
| endocrine and | iobenguane I 131 | NET |
| neuroendocrine | (Azedra) | |
| tumors | | |
| colorectal | ipilimumab (Yervoy) | CTLA4 |
| esophageal | ipilimumab (Yervoy) | CTLA4 |
| kidney | ipilimumab (Yervoy) | CTLA4 |
| liver and bile duct | ipilimumab (Yervoy) | CTLA4 |
| lung | ipilimumab (Yervoy) | CTLA4 |
| malignant | ipilimumab (Yervoy) | CTLA4 |
| mesothelioma | | |
| skin | ipilimumab (Yervoy) | CTLA4 |
| multiple myeloma | isatuximab-irfc | CD38 |
| | (Sarclisa) | |
| leukemia | ivosidenib (Tibsovo) | IDH1 |
| multiple myeloma | ixazomib citrate | proteasome |
| | (Ninlaro) | |
| endocrine and | lanreotide acetate | SSTR |
| neuroendocrine | (Somatuline Depot) | |
| tumors | | |
| Breast | lapatinib ditosylate | EGFR |
| | (Tykerb) | |
| solid tumors | larotrectinib sulfate | TRKA + TrkB + TrkC |
| anywhere in the | (Vitrakvi) | |
| body | | |
| endometrial | lenvatinib mesylate | FGFR1 + FGFR2 + FGFR3 + FGFR4 + PDGFRα + |
| | (Lenvima) | RET + |
| | | VEGFR1 + VEGFR2 + VEGFR3 + c-Kit |
| kidney | lenvatinib mesylate | FGFR1 + FGFR2 + FGFR3 + FGFR4 + PDGFRα + |
| | (Lenvima) | RET + |
| | | VEGFR1 + VEGFR2 + VEGFR3 + c-Kit |
| liver and bile duct | lenvatinib mesylate | FGFR1 + FGFR2 + FGFR3 + FGFR4 + PDGFRα + |
| | (Lenvima) | RET + |
| | | VEGFR1 + VEGFR2 + VEGFR3 + c-Kit |
| thyroid | lenvatinib mesylate | FGFR1 + FGFR2 + FGFR3 + FGFR4 + PDGFRα + |
| | (Lenvima) | RET + |
| | | VEGFR1 + VEGFR2 + VEGFR3 + c-Kit |
| Breast | letrozole | CDK4/ER |
| | (Femara)/Ribociclib | |
| | Succinate | |
| lymphoma | lisocabtagene | CD19 |
| | maraleucel (Breyanzi) | |
| lymphoma | loncastuximab | CD19 + DNA |
| | tesirine-lpyl | |
| | (Zynlonta) | |
| lung | lorlatinib (Lorbrena) | ALK + ROS1 |
| prostate | lutetium Lu 177 | PSMA |
| | vipivotide tetraxetan | |
| | (Pluvicto) | |
| endocrine and | lutetium Lu 177- | SSTR2 |
| neuroendocrine | dotatate (Lutathera) | |
| tumors | | |
| Breast | margetuximab-cmkb | HER2 |
| | (Margenza) | |
| leukemia | midostaurin (Rydapt) | FLT3 + PDGFR + PKC + Syk + VEGFR2 + c-Kit |
| systemic | midostaurin (Rydapt) | FLT3 + PDGFR + PKC + Syk + VEGFR2 + c-Kit |
| mastocytosis | | |
| ovarian epithelial, | mirvetuximab | FOLR1 + Tubulin |
| fallopian tube, and | soravtansine-gynx | |
| primary peritoneal | (Elahere) | |
| lung | mobocertinib | EGFR exon 20 + HER2 exon 20 |
| | succinate (Exkivity) | |
| lymphoma | mogamulizumab-kpkc | CCR4 |
| | (Poteligeo) | |
| lymphoma | mosunetuzumab- | CD20, CD3 |
| | axgb (Lunsumio) | |
| leukemia | moxetumomab | CD22 |
| | pasudotox- | |
| | tdfk (Lumoxiti) | |
| neuroblastoma | naxitamab-gqgk | GD2 |
| | (Danyelza) | |
| lung | necitumumab | EGFR |
| | (Portrazza) | |
| Breast | neratinib maleate | EGFR, HER2, HER4 |
| | (Nerlynx) | |
| leukemia | nilotinib (Tasigna) | Bcr-Abl + CSF-1R + DDR1 + PDGFR + c-Kit |
| ovarian epithelial, | niraparib tosylate | PARP1 + PARP2 |
| fallopian tube, and | monohydrate (Zejula) | |
| primary peritoneal | | |
| bladder | nivolumab (Opdivo) | PD-1 |
| colorectal | nivolumab (Opdivo) | PD-1 |
| esophageal | nivolumab (Opdivo) | PD-1 |
| kidney | nivolumab (Opdivo) | PD-1 |
| liver and bile duct | nivolumab (Opdivo) | PD-1 |
| lung | nivolumab (Opdivo) | PD-1 |
| lymphoma | nivolumab (Opdivo) | PD-1 |
| malignant | nivolumab (Opdivo) | PD-1 |
| mesothelioma | | |
| skin | nivolumab (Opdivo) | PD-1 |
| stomach (gastric) | nivolumab (Opdivo) | PD-1 |
| skin | nivolumab and | LAG3 + PD-1 |
| | relatlimab-rmbw | |
| | (Opdualag) | |
| leukemia | obinutuzumab | CD20 |
| | (Gazyva) | |
| lymphoma | obinutuzumab | CD20 |
| | (Gazyva) | |
| leukemia | ofatumumab | CD20 |
| | (Arzerra) | |
| ovarian epithelial, | olaparib (Lynparza) | PARP1, PARP2, PARP3 |
| fallopian tube, and | | |
| primary peritoneal | | |
| Breast | olaparib (Lynparza) | PARP1, PARP2, PARP3 |
| pancreatic | olaparib (Lynparza) | PARP1, PARP2, PARP3 |
| prostate | olaparib (Lynparza) | PARP1, PARP2, PARP3 |
| leukemia | olutasidenib | IDH1 |
| | (Rezlidhia) | |
| lung | osimertinib mesylate | EGFR + EGFR T790M + EGFR exon 21 L858R |
| | (Tagrisso) | mutation + |
| | | EGFR-Ex19del |
| myelodysplastic | pacritinib citrate | CSF-1R + FLT3 + IRAK1 + JAK2 |
| and | (Vonjo) | |
| myeloproliferative | | |
| disorders | | |
| Breast | palbociclib (Ibrance) | CDK4, CDK6 |
| colorectal | panitumumab | EGFR |
| | (Vectibix) | |
| kidney | pazopanib | FGFR1 + FGFR3 + Flt3L + ITK + LCK + PDGFRα + |
| | hydrochloride (Votrient) | PDGFRβ + |
| | | VEGFR1 + VEGFR2 + VEGFR3 + c-Kit |
| soft tissue sarcoma | pazopanib | FGFR1 + FGFR3 + Flt3L + ITK + LCK + PDGFRα + |
| | hydrochloride | PDGFRβ + |
| | (Votrient) | VEGFR1 + VEGFR2 + VEGFR3 + c-Kit |
| Breast | pembrolizumab | PD-1 |
| | (Keytruda) | |
| Cervical | pembrolizumab | PD-1 |
| | (Keytruda) | |
| colorectal | pembrolizumab | PD-1 |
| | (Keytruda) | |
| endometrial | pembrolizumab | PD-1 |
| | (Keytruda) | |
| esophageal | pembrolizumab | PD-1 |
| | (Keytruda) | |
| kidney | pembrolizumab | PD-1 |
| | (Keytruda) | |
| liver and bile duct | pembrolizumab | PD-1 |
| | (Keytruda) | |
| lung | pembrolizumab | PD-1 |
| | (Keytruda) | |
| lymphoma | pembrolizumab | PD-1 |
| | (Keytruda) | |
| skin | pembrolizumab | PD-1 |
| | (Keytruda) | |
| solid tumors | pembrolizumab | PD-1 |
| anywhere in the | (Keytruda) | |
| body | | |
| stomach (gastric) | pembrolizumab | PD-1 |
| | (Keytruda) | |
| leukemia | pemigatinib (Pemazyre) | FGFR1, FGFR2, FGFR3, FGFR4 |
| liver and bile duct | pemigatinib | FGFR1, FGFR2, FGFR3, FGFR4 |
| | (Pemazyre) | |
| lymphoma | pemigatinib (Pemazyre) | FGFR1, FGFR2, FGFR3, FGFR4 |
| myelodysplastic | pemigatinib (Pemazyre) | FGFR1, FGFR2, FGFR3, FGFR4 |
| and | | |
| myeloprolifertive | | |
| disorders | | |
| Breast | pertuzumab (Perjeta) | HER2 |
| Breast | pertuzumab, | HER2, Hyaluronic acid |
| | trastuzumab, and | |
| | hyaluronidase-zzxf | |
| | (Phesgo) | |
| giant cell tumor | pexidartinib | CSF-1R + FLT3 + c-Kit |
| | hydrochloride | |
| | (Turalio) | |
| lymphoma | pirtobrutinib | BTK C481S |
| | (Jaypirca) | |
| lymphoma | polatuzumab vedotin- | CD79B + Tubulin |
| | piiq (Polivy) | |
| leukemia | ponatinib | Bcr-Abl + Protein-tyrosine kinases |
| | hydrochloride | |
| | (Iclusig) | |
| lymphoma | pralatrexate (Folotyn) | Bcr-Abl + Protein-tyrosine kinases |
| lung | pralsetinib (Gavreto) | RET |
| thyroid | pralsetinib (Gavreto) | RET |
| prostate | radium 223 dichloride | DNA |
| | (Xofigo) | |
| colorectal | ramucirumab | VEGFR2 |
| | (Cyramza) | |
| Gastric | ramucirumab | VEGFR2 |
| | (Cyramza) | |
| liver and bile duct | ramucirumab | VEGFR2 |
| | (Cyramza) | |
| lung | ramucirumab | VEGFR2 |
| | (Cyramza) | |
| stomach (gastric) | ramucirumab | VEGFR2 |
| | (Cyramza) | |
| colorectal | regorafenib (Stivarga) | Abl family, BRAF, BRAF V600E, CRAF, CSF-1R, |
| | | DDR2, |
| | | EphA2, FGFR1, FGFR2, FRK, MAPK11, PDGFRa, |
| | | PDGFRb, |
| | | RET, TRKA, Tie-2, VEGFR1, VEGFR2, VEGFR3, c- |
| | | kit |
| Gastrointestinal | regorafenib (Stivarga) | Abl family, BRAF, BRAF V600E, CRAF, CSF-1R, |
| | | DDR2, |
| | | EphA2, FGFR1, FGFR2, FRK, MAPK11, PDGFRa, |
| | | PDGFRb, |
| | | RET, TRKA, Tie-2, VEGFR1, VEGFR2, VEGFR3, c- |
| | | kit |
| liver and bile duct | regorafenib (Stivarga) | Abl family, BRAF, BRAF V600E, CRAF, CSF-1R, |
| | | DDR2, |
| | | EphA2, FGFR1, FGFR2, FRK, MAPK11, PDGFRa, |
| | | PDGFRb, |
| | | RET, TRKA, Tie-2, VEGFR1, VEGFR2, VEGFR3, c- |
| | | kit |
| skin | retifanlimab-dlwr | PD-1 |
| | (Zynyz) | |
| Breast | ribociclib (Kisqali) | CDK4, CDK6 |
| gastrointestinal | ripretinib (Qinlock) | EGFR + PDGFRα + c-Kit |
| leukemia | rituximab (Rituxan) | CD20 + Hyaluronic acid |
| lymphoma | rituximab (Rituxan) | CD20 + Hyaluronic acid |
| leukemia | rituximab and | CD20 + Hyaluronic acid |
| | hyaluronidase human | |
| | (Rituxan Hycela) | |
| lymphoma | rituximab and | CD20 + Hyaluronic acid |
| | hyaluronidase human | |
| | (Rituxan Hycela) | |
| lymphoma | romidepsin (Istodax) | HDAC |
| ovarian epithelial, | rucaparib | PARP1 + PARP2 + PARP3 |
| fallopian tube, and | camsylate (Rubraca) | |
| primary peritoneal | | |
| prostate | rucaparib camsylate | PARP1 + PARP2 + PARP3 |
| | (Rubraca) | |
| myelodysplastic | ruxolitinib phosphate | JAK1 + JAK2 |
| and | (Jakafi) | |
| myeloproliferative | | |
| disorders | | |
| Breast | sacituzumab | TOP1 |
| | govitecan-hziy | |
| | (Trodelvy) | |
| lymphoma | selinexor (Xpovio) | XPO1 |
| multiple myeloma | selinexor (Xpovio) | XPO1 |
| lung | selpercatinib | RET |
| | (Retevmo) | |
| solid tumors | selpercatinib | RET |
| anywhere in the | (Retevmo) | |
| body | | |
| thyroid | selpercatinib | RET |
| | (Retevmo) | |
| plexiform | selumetinib sulfate | MEK1 + MEK2 |
| neurofibroma | (Koselugo) | |
| soft tissue sarcoma | sirolimus protein- | MUT + mTOR |
| | bound particles | |
| | (Fyarro) | |
| skin | sonidegib (Odomzo) | SMO |
| kidney | sorafenib tosylate | BRAF inhibitors, CRAF inhibitors, FLT3 + |
| | (Nexavar) | PDGFRβ + |
| | | RET + VEGFR1 + VEGFR2 + VEGFR3 + c-Kit |
| liver and bile duct | sorafenib tosylate | BRAF + CRAF + FLT3 + PDGFRβ + RET + VEGFR1 + |
| | (Nexavar) | VEGFR2 + VEGFR3 + c-Kit |
| thyroid | sorafenib tosylate | BRAF + CRAF + FLT3 + PDGFRβ + RET + VEGFR1 + |
| | (Nexavar) | VEGFR2 + VEGFR3 + c-Kit |
| lung | sotorasib (Lumakras) | KRAS G12C |
| Gastrointestinal | sunitinib malate | PDGFR + RTK + VEGFR |
| | (Sutent) | |
| kidney | sunitinib malate | PDGFR + RTK + VEGFR |
| | (Sutent) | |
| pancreatic | sunitinib malate | PDGFR + RTK + VEGFR |
| | (Sutent) | |
| lymphoma | tafasitamab-cxix | CD19 |
| | (Monjuvi) | |
| leukemia | tagraxofusp-erzs | CD123 |
| | (Elzonris) | |
| Breast | talazoparib tosylate | PARP1, PARP2 |
| | (Talzenna) | |
| Breast | tamoxifen citrate | ER |
| | (Soltamox) | |
| lymphoma | tazemetostat | EZH2 |
| | hydrobromide | |
| | (Tazverik) | |
| soft tissue sarcoma | tazemetostat | EZH2 |
| | hydrobromide | |
| | (Tazverik) | |
| skin | tebentafusp-tebn | CD3 + gp100 |
| | (Kimmtrak) | |
| multiple myeloma | teclistamab-cqyv | CD3 + gp100 |
| | (Tecvayli) | |
| kidney | temsirolimus (Torisel) | CD3 + gp100 |
| lung | tepotinib | MET |
| | hydrochloride | |
| | (Tepmetko) | |
| leukemia | tisagenlecleucel | CD19 |
| | (Kymriah) | |
| lymphoma | tisagenlecleucel | CD19 |
| | (Kymriah) | |
| Cervical | tisotumab vedotin- | Tubulin, tissue factor |
| | tftv (Tivdak) | |
| kidney | tivozanib | (VEGFR)-1, VEGFR-2 and VEGFR-3, c-kit, and |
| | hydrochloride | PDGFR β |
| | (Fotivda) | |
| Breast | toremifene (Fareston) | ER |
| Brain | trametinib (Mekinist) | MEK1 and MEK2 |
| lung | trametinib dimethyl | MEK1 and MEK2 |
| | sulfoxide (Mekinist) | |
| skin | trametinib dimethyl | MEK1 and MEK2 |
| | sulfoxide (Mekinist) | |
| solid tumors | trametinib dimethyl | MEK1 and MEK2 |
| anywhere in the | sulfoxide (Mekinist) | |
| body | | |
| thyroid | trametinib dimethyl | MEK1 and MEK2 |
| | sulfoxide (Mekinist) | |
| Breast | trastuzumab | HER2 |
| | (Herceptin) | |
| esophageal | trastuzumab | HER2 |
| | (Herceptin) | |
| stomach (gastric) | trastuzumab | HER2 |
| | (Herceptin) | |
| liver and bile duct | tremelimumab-actl | CTLA-4, CD80, and CD86 |
| | (Imjudo) | |
| lung | tremelimumab-actl | CTLA-4, CD80, and CD86 |
| | (Imjudo) | |
| Breast | tucatinib (Tukysa) | HER2 |
| colorectal | tucatinib (Tukysa) | HER2 |
| thyroid | vandetanib (Caprelsa) | VEGFR-2, EGFR, RET |
| skin | vemurafenib | BRAF, BRAF V600E, CRAF, ARAF, SRMS, ACK1, |
| | (Zelboraf) | MAP4K5, FGR |
| leukemia | venetoclax | BCL-2 |
| | (Venclexta) | |
| lymphoma | venetoclax | BCL-2 |
| | (Venclexta) | |
| lymphoma | vorinostat (Zolinza) | HDAC1, HDAC2 and HDAC3 (Class I) and |
| | | HDAC6 (Class II) |
| leukemia | zanubrutinib | BTK |
| | (Brukinsa) | |
| lymphoma | zanubrutinib | BTK |
| | (Brukinsa) | |
| colorectal | ziv-aflibercept | PGF, VEGF-A |
| | (Zaltrap) | |

Patients with germline or somatic HRD may be candidates for targeted therapies, including DNA damage response (DDR) inhibitors, such as poly (ADP-ribose) polymerase (PARP) inhibitors (PARPi) [Fong et al., “Poly (ADP)-ribose polymerase inhibition: frequent durable responses in BRCA carrier ovarian cancer correlating with platinum-free interval,” J Clin Oncol, 28:2512-9 (2010); Audeh et al., “Oral poly (ADP-ribose) polymerase inhibitor olaparib in patients with BRCA1 or BRCA2 mutations and recurrent ovarian cancer: a proof-of-concept trial,” Lancet, 376:245-51 (2010)].

In some embodiments, a subject having HRD as determined by any of the methods disclosed may be administered a targeted therapy. The targeted therapy may comprise a PARP inhibitor. Examples of PARP inhibitors that may be administered include one or more of: VELIPARIB, OLAPARIB, TALAZOPARIB, RUCAPARIB, NIRAPARIB, PAMIPARIB, CEP 9722 (Cephalon), E7016 (Eisai), E7449 (Eisai, a PARP ½ and tankyrase ½ inhibitor), or 3-Aminobenzamide. In some embodiments, the targeted therapy may comprise at least one base excision repair (BER) inhibitor. For example, OLAPARIB may inhibit BER. In certain embodiments, the targeted therapy may comprise combination of a PARP inhibitor and radiotherapy. In an embodiment, the combination of a PARP inhibitor and radiotherapy would permit the PARP inhibitor to lead to formation of double strand breaks from the single-strand breaks generated by the radiotherapy in tumor tissue (e.g., tissue with BRCA1/BRCA2 mutations). This combination can provide more powerful therapy per radiation dose.

In some embodiments, the therapies are PARP inhibitors, such as Olaparib (Lynparza), Rucaparib (Rubraca), Niraparib (Zejula), and Talazoparib (Talzenna). These may be used for treating mutations in BRCA1, BRCA2, ATM, BARD1, BRIP1, CDK12, CHEK1, CHEK2, FANCL, PALB2, RAD51B, RAD51 C, RAD51D and RAD54L alterations, and/or for associated Homologous Recombination Repair (HRR) genes.

In certain implementations, the therapy administered to a subject may comprise at least one chemotherapy drug. In some implementations, the chemotherapy drug may comprise alkylating agents (for example, but not limited to, Chlorambucil, Cyclophosphamide, Cisplatin and Carboplatin), nitrosoureas (for example, but not limited to, Carmustine and Lomustine), anti-metabolites (for example, but not limited to, Fluorauracil, Methotrexate and Fludarabine), plant alkaloids and natural products (for example, but not limited to, Vincristine, Paclitaxel and Topotecan), anti-tumor antibiotics (for example, but not limited to, Bleomycin, Doxorubicin and Mitoxantrone), hormonal agents (for example, but not limited to, Prednisone, Dexamethasone, Tamoxifen and Leuprolide) and biological response modifiers (for example, but not limited to, Herceptin and Avastin, Erbitux and Rituxan). In some implementations, the chemotherapy administered to a subject may comprise FOLFOX or FOLFIRI. Typically, therapies include at least one immunotherapy (or an immunotherapeutic agent). Immunotherapy refers generally to methods of enhancing an immune response against a given cancer type. In certain implementations, immunotherapy refers to methods of enhancing a T cell response against a tumor or cancer.

In some implementations, the immunotherapy or immunotherapeutic agents targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain implementations, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain implementations, the inhibitory immune checkpoint molecule is CTLA4 or PD-1. In other implementations, the inhibitory immune checkpoint molecule is a ligand for PD-1, such as PD-L1 or PD-L2. In other implementations, the inhibitory immune checkpoint molecule is a ligand for CTLA4, such as CD80 or CD86. In other implementations, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain implementations, the immunotherapy or immunotherapeutic agent is an antagonist of an inhibitory immune checkpoint molecule. In certain implementations, the inhibitory immune checkpoint molecule is PD-1. In certain implementations, the inhibitory immune checkpoint molecule is PD-L1. In certain implementations, the antagonist of the inhibitory immune checkpoint molecule is an antibody (e.g., a monoclonal antibody). In certain implementations, the antibody or monoclonal antibody is an anti-CTLA4, anti-PD-1, anti-PD-L1, or anti-PD-L2 antibody. In certain implementations, the antibody is a monoclonal anti-PD-1 antibody. In some implementations, the antibody is a monoclonal anti-PD-L1 antibody. In certain implementations, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain implementations, the anti-PD-1 antibody is one or more of pembrolizumab (Keytruda®) or nivolumab (Opdivo®). In certain implementations, the anti-CTLA4 antibody is ipilimumab (Yervoy®). In certain implementations, the anti-PD-L1 antibody is one or more of atezolizumab (Tecentriq®), avelumab (Bavencio®), or durvalumab (Imfinzi®).

In certain implementations, the immunotherapy or immunotherapeutic agent is an antagonist (e.g., antibody) against CD80, CD86, LAG3, KIR, TIM3, GAL9, or A2aR. In other implementations, the antagonist is a soluble version of the inhibitory immune checkpoint molecule, such as a soluble fusion protein comprising the extracellular domain of the inhibitory immune checkpoint molecule and an Fc domain of an antibody. In certain implementations, the soluble fusion protein comprises the extracellular domain of CTLA4, PD-1, PD-L1, or PD-L2. In some implementations, the soluble fusion protein comprises the extracellular domain of CD80, CD86, LAG3, KIR, TIM3, GAL9, or A2aR. In one implementation, the soluble fusion protein comprises the extracellular domain of PD-L2 or LAG3.

In certain implementations, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain implementations, the immune checkpoint molecule is a co-stimulatory molecule selected from CD28, inducible T cell co-stimulator (ICOS), CD137, OX40, or CD27. In other implementations, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain implementations, the immunotherapy or immunotherapeutic agent is an agonist of a co-stimulatory checkpoint molecule. In certain implementations, the agonist of the co-stimulatory checkpoint molecule is an agonist antibody and preferably is a monoclonal antibody. In certain implementations, the agonist antibody or monoclonal antibody is an anti-CD28 antibody. In other implementations, the agonist antibody or monoclonal antibody is an anti-ICOS, anti-CD137, anti-OX40, or anti-CD27 antibody. In other implementations, the agonist antibody or monoclonal antibody is an anti-CD80, anti-CD86, anti-B7RP1, anti-B7-H3, anti-B7-H4, anti-CD137L, anti-OX40L, or anti-CD70 antibody.

Therapeutic options for treating specific genetic-based diseases, disorders, or conditions, other than cancer, are generally well-known to those of ordinary skill in the art and will be apparent given the particular disease, disorder, or condition under consideration.

In certain implementations, the customized therapies described herein are typically administered parenterally (e.g., intravenously or subcutaneously). Pharmaceutical compositions containing the immunotherapeutic agent are typically administered intravenously. Certain therapeutic agents are administered orally. However, customized therapies (e.g., immunotherapeutic agents, etc.) may also be administered by any method known in the art, including, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

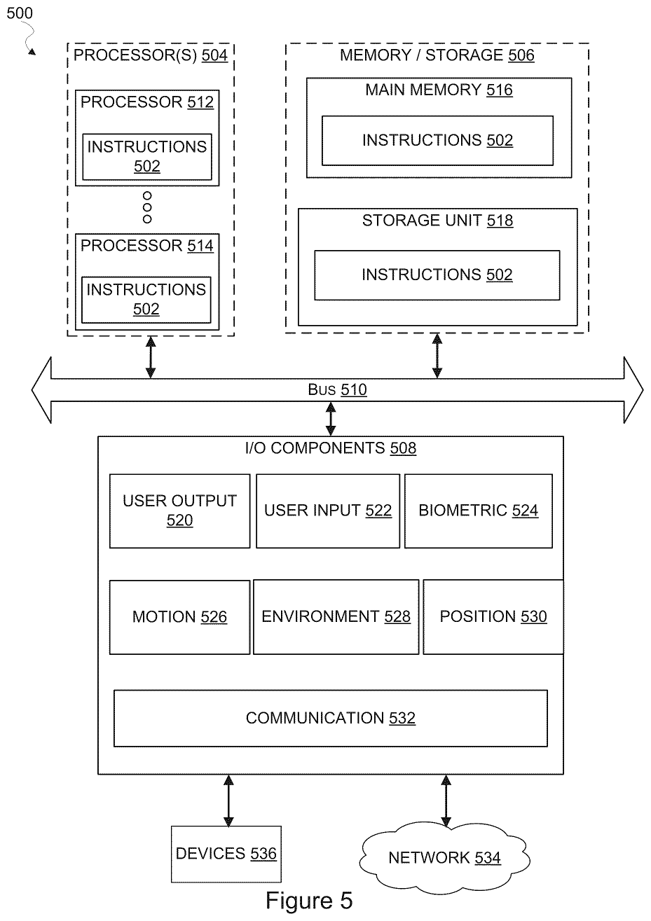

FIG. 5 is a block diagram illustrating components of a machine 500, according to some example implementations, able to read instructions from a machine-readable medium (e.g., a machine-readable storage medium) and perform any one or more of the methodologies discussed herein. Specifically, FIG. 5 shows a diagrammatic representation of the machine 500 in the example form of a computer system, within which instructions 502 (e.g., software, a program, an application, an applet, an app, or other executable code) for causing the machine 500 to perform any one or more of the methodologies discussed herein may be executed. As such, the instructions 502 may be used to implement modules or components described herein. The instructions 502 transform the general, non-programmed machine 500 into a particular machine 500 programmed to carry out the described and illustrated functions in the manner described. In alternative implementations, the machine 500 operates as a standalone device or may be coupled (e.g., networked) to other machines. In a networked deployment, the machine 500 may operate in the capacity of a server machine or a client machine in a server-client network environment, or as a peer machine in a peer-to-peer (or distributed) network environment. The machine 500 may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 502, sequentially or otherwise, that specify actions to be taken by machine 500. Further, while only a single machine 500 is illustrated, the term “machine” shall also be taken to include a collection of machines that individually or jointly execute the instructions 502 to perform any one or more of the methodologies discussed herein.

The machine 500 may include processors 504, memory/storage 506, and I/O components 508, which may be configured to communicate with each other such as via a bus 510. In an example implementation, the processors 504 (e.g., a central processing unit (CPU), a reduced instruction set computing (RISC) processor, a complex instruction set computing (CISC) processor, a graphics processing unit (GPU), a digital signal processor (DSP), an application-specific integrated circuit (ASIC), a radio-frequency integrated circuit (RFIC), another processor, or any suitable combination thereof) may include, for example, a processor 512 and a processor 514 that may execute the instructions 502. The term “processor” is intended to include multi-core processors 504 that may comprise two or more independent processors (sometimes referred to as “cores”) that may execute instructions 502 contemporaneously. Although FIG. 5 shows multiple processors 504, the machine 500 may include a single processor 512 with a single core, a single processor 512 with multiple cores (e.g., a multi-core processor), multiple processors 512, 514 with a single core, multiple processors 512, 514 with multiple cores, or any combination thereof.

The memory/storage 506 may include memory, such as a main memory 516, or other memory storage, and a storage unit 518, both accessible to the processors 504 such as via the bus 510. The storage unit 518 and main memory 516 store the instructions 502 embodying any one or more of the methodologies or functions described herein. The instructions 502 may also reside, completely or partially, within the main memory 516, within the storage unit 518, within at least one of the processors 504 (e.g., within the processor's cache memory), or any suitable combination thereof, during execution thereof by the machine 500. Accordingly, the main memory 516, the storage unit 518, and the memory of processors 504 are examples of machine-readable media.

The I/O components 508 may include a wide variety of components to receive input, provide output, produce output, transmit information, exchange information, capture measurements, and so on. The specific I/O components 508 that are included in a particular machine 500 will depend on the type of machine. For example, portable machines such as mobile phones will likely include a touch input device or other such input mechanisms, while a headless server machine will likely not include such a touch input device. It will be appreciated that the I/O components 508 components 508 may include many other components that are not shown in FIG. 5. The I/O components 508 are grouped according to functionality merely for simplifying the following discussion and the grouping is in no way limiting. In various example implementations, the I/O components 508 components 508 may include user output components 520 and user input components 522. The user output components 520 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The user input components 522 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or other pointing instrument), tactile input components (e.g., a physical button, a touch screen that provides location or force of touches or touch gestures, or other tactile input components), audio input components (e.g., a microphone), and the like.

In further example implementations, the I/O components 508 components 508 may include biometric components 524, motion components 526, environmental components 528, or position components 530 among a wide array of other components. For example, the biometric components 524 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram based identification), and the like. The motion components 526 may include acceleration sensor components (e.g., accelerometer), gravitation sensor components, rotation sensor components (e.g., gyroscope), and so forth. The environmental components 528 may include, for example, illumination sensor components (e.g., photometer), temperature sensor components (e.g., one or more thermometer that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 530 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 508 may include communication components 532 operable to couple the machine 500 to a network 534 or devices 536. For example, the communication components 532 may include a network interface component or other suitable device to interface with the network 534. In further examples, communication components 532 may include wired communication components, wireless communication components, cellular communication components, near field communication (NFC) components, Bluetooth® components (e.g., Bluetooth® Low Energy), Wi-Fi® components, and other communication components to provide communication via other modalities. The devices 536 may be another machine 500 or any of a wide variety of peripheral devices (e.g., a peripheral device coupled via a USB).

Moreover, the communication components 532 may detect identifiers or include components operable to detect identifiers. For example, the communication components 532 may include radio frequency identification (RFID) tag reader components, NFC smart tag detection components, optical reader components (e.g., an optical sensor to detect one-dimensional barcodes such as Universal Product Code (UPC) barcode, multi-dimensional barcodes such as Quick Response (QR) code, Aztec code, Data Matrix, Dataglyph, MaxiCode, PDF417, Ultra Code, UCC RSS-2D barcode, and other optical codes), or acoustic detection components (e.g., microphones to identify tagged audio signals). In addition, a variety of information may be derived via the communication components 532, such as location via Internet Protocol (IP) geo-location, location via Wi-Fi® signal triangulation, location via detecting an NFC beacon signal that may indicate a particular location, and so forth.

As used herein, “component” refers to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example implementations, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein.

A hardware component may also be implemented mechanically, electronically, or any suitable combination thereof. For example, a hardware component may include dedicated circuitry or logic that is permanently configured to perform certain operations. A hardware component may be a special-purpose processor, such as a field-programmable gate array (FPGA) or an ASIC. A hardware component may also include programmable logic or circuitry that is temporarily configured by software to perform certain operations. For example, a hardware component may include software executed by a general-purpose processor 504 or other programmable processor. Once configured by such software, hardware components become specific machines (or specific components of a machine 500) uniquely tailored to perform the configured functions and are no longer general-purpose processors 504. It will be appreciated that the decision to implement a hardware component mechanically, in dedicated and permanently configured circuitry, or in temporarily configured circuitry (e.g., configured by software) may be driven by cost and time considerations. Accordingly, the phrase “hardware component” (or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering implementations in which hardware components are temporarily configured (e.g., programmed), each of the hardware components need not be configured or instantiated at any one instance in time. For example, where a hardware component comprises a general-purpose processor 504 configured by software to become a special-purpose processor, the general-purpose processor 504 may be configured as respectively different special-purpose processors (e.g., comprising different hardware components) at different times. Software accordingly configures a particular processor 512, 514 or processors 504, for example, to constitute a particular hardware component at one instance of time and to constitute a different hardware component at a different instance of time.

Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In implementations in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output.

Hardware components may also initiate communications with input or output devices, and can operate on a resource (e.g., a collection of information). The various operations of example methods described herein may be performed, at least partially, by one or more processors 504 that are temporarily configured (e.g., by software) or permanently configured to perform the relevant operations. Whether temporarily or permanently configured, such processors 504 may constitute processor-implemented components that operate to perform one or more operations or functions described herein. As used herein, “processor-implemented component” refers to a hardware component implemented using one or more processors 504. Similarly, the methods described herein may be at least partially processor-implemented, with a particular processor 512, 514 or processors 504 being an example of hardware. For example, at least some of the operations of a method may be performed by one or more processors 504 or processor-implemented components. Moreover, the one or more processors 504 may also operate to support performance of the relevant operations in a “cloud computing” environment or as a “software as a service” (SaaS). For example, at least some of the operations may be performed by a group of computers (as examples of machines 500 including processors 504), with these operations being accessible via a network 534 (e.g., the Internet) and via one or more appropriate interfaces (e.g., an API). The performance of certain of the operations may be distributed among the processors, not only residing within a single machine 500, but deployed across a number of machines. In some example implementations, the processors 504 or processor-implemented components may be located in a single geographic location (e.g., within a home environment, an office environment, or a server farm). In other example implementations, the processors 504 or processor-implemented components may be distributed across a number of geographic locations.

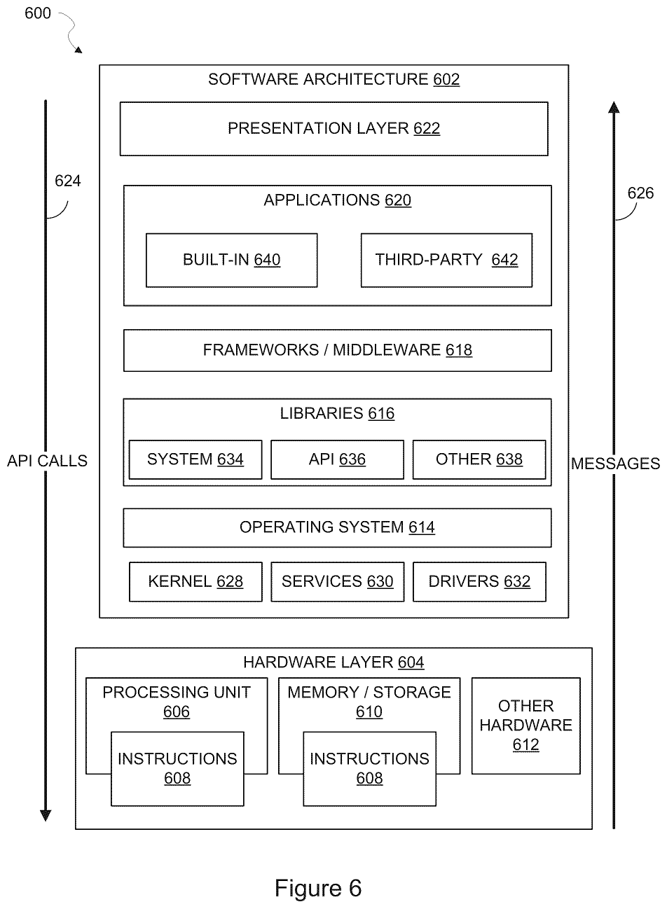

FIG. 6 is a block diagram illustrating system 600 that includes an example software architecture 602, which may be used in conjunction with various hardware architectures herein described. FIG. 6 is a non-limiting example of a software architecture, and it will be appreciated that many other architectures may be implemented to facilitate the functionality described herein. The software architecture 602 may execute on hardware such as machine 500 of FIG. 5 that includes, among other things, processors 504, memory/storage 506, and input/output (I/O) components 508. A representative hardware layer 604 is illustrated and can represent, for example, the machine 500 of FIG. 5. The representative hardware layer 604 includes a processing unit 606 having associated executable instructions 608. Executable instructions 608 represent the executable instructions of the software architecture 602, including implementation of the methods, components, and so forth described herein. The hardware layer 604 also includes at least one of memory or storage modules memory/storage 610, which also have executable instructions 608. The hardware layer 604 may also comprise other hardware 612.

In the example architecture of FIG. 6, the software architecture 602 may be conceptualized as a stack of layers where each layer provides particular functionality. For example, the software architecture 602 may include layers such as an operating system 614, libraries 616, frameworks/middleware 618, applications 620, and a presentation layer 622. Operationally, the applications 620 or other components within the layers may invoke API calls 624 through the software stack and receive messages 626 in response to the API calls 624. The layers illustrated are representative in nature and not all software architectures have all layers. For example, some mobile or special purpose operating systems may not provide a frameworks/middleware 618, while others may provide such a layer. Other software architectures may include additional or different layers.

The operating system 614 may manage hardware resources and provide common services. The operating system 614 may include, for example, a kernel 628, services 630, and drivers 632. The kernel 628 may act as an abstraction layer between the hardware and the other software layers. For example, the kernel 628 may be responsible for memory management, processor management (e.g., scheduling), component management, networking, security settings, and so on. The services 630 may provide other common services for the other software layers. The drivers 632 are responsible for controlling or interfacing with the underlying hardware. For instance, the drivers 632 include display drivers, camera drivers, Bluetooth® drivers, flash memory drivers, serial communication drivers (e.g., Universal Serial Bus (USB) drivers), Wi-Fi® drivers, audio drivers, power management drivers, and so forth depending on the hardware configuration.

The libraries 616 provide a common infrastructure that is used by at least one of the applications 620, other components, or layers. The libraries 616 provide functionality that allows other software components to perform tasks in an easier fashion than to interface directly with the underlying operating system 614 functionality (e.g., kernel 628, services 630, drivers 632). The libraries 616 may include system libraries 634 (e.g., C standard library) that may provide functions such as memory allocation functions, string manipulation functions, mathematical functions, and the like. In addition, the libraries 616 may include API libraries 636 such as media libraries (e.g., libraries to support presentation and manipulation of various media format such as MPEG4, H.264, MP3, AAC, AMR, JPG, PNG), graphics libraries (e.g., an OpenGL framework that may be used to render two-dimensional and three-dimensional in a graphic content on a display), database libraries (e.g., SQLite that may provide various relational database functions), web libraries (e.g., WebKit that may provide web browsing functionality), and the like. The libraries 616 may also include a wide variety of other libraries 638 to provide many other APIs to the applications 620 and other software components/modules.

The frameworks/middleware 618 (also sometimes referred to as middleware) provide a higher-level common infrastructure that may be used by the applications 620 or other software components/modules. For example, the frameworks/middleware 618 may provide various graphical user interface functions, high-level resource management, high-level location services, and so forth. The frameworks/middleware 618 may provide a broad spectrum of other APIs that may be utilized by the applications 620 or other software components/modules, some of which may be specific to a particular operating system 614 or platform.

The applications 620 include built-in applications 640 and third-party applications 642. Examples of representative built-in applications 640 may include, but are not limited to, a contacts application, a browser application, a book reader application, a location application, a media application, a messaging application, or a game application. Third-party applications 642 may include an application developed using the ANDROID™ or IOS™ software development kit (SDK) by an entity other than the vendor of the particular platform, and may be mobile software running on a mobile operating system such as IOS™, ANDROID™, WINDOWS® Phone, or other mobile operating systems. The third-party applications 642 may invoke the API calls 624 provided by the mobile operating system (such as operating system 614) to facilitate functionality described herein.

The applications 620 may use built-in operating system functions (e.g., kernel 628, services 630, drivers 632), libraries 616, and frameworks/middleware 618 to create UIs to interact with users of the system. Alternatively, or additionally, in some systems, interactions with a user may occur through a presentation layer, such as presentation layer 622. In these systems, the application/component “logic” can be separated from the aspects of the application/component that interact with a user.

At least some of the processes described herein can be embodied in computer-readable instructions for execution by one or more processors such that the operations of the processes may be performed in part or in whole by the functional components of one or more computer systems. Accordingly, computer-implemented processes described herein are by way of example with reference thereto, in some situations. However, in other implementations, at least some of the operations of the computer-implemented processes described herein can be deployed on various other hardware configurations. The computer-implemented processes described herein are therefore not intended to be limited to the systems and configurations described with respect to FIGS. 5 and 6 and can be implemented in whole, or in part, by one or more additional system and/or components.

Although the flowcharts described herein can show operations as a sequential process, many of the operations can be performed in parallel or concurrently. In addition, the order of the operations can be re-arranged. A process is terminated when its operations are completed. A process can correspond to a method, a procedure, an algorithm, etc. The operations of methods may be performed in whole or in part, can be performed in conjunction with some or all of the operations in other methods, and can be performed by any number of different systems, such as the systems described herein, or any portion thereof, such as a processor included in any of the systems.

### Example

143 Samples were collected and methylation data was determined for the samples, where the methylation data indicated counts of methylated cytosines in CG regions of classification regions and control regions. Samples that generated less than 50,000 molecule counts were removed. The samples were collected from subjects having a number of different types of cancer. For example, sample were collected from subjects in which bladder cancer was present, breast cancer was present, colorectal cancer was present, gastric cancer was present, lung cancer was present, ovarian cancer was present, pancreatic cancer was present, and prostate cancer was present. A portion of the subjects in which cancer was present were negative for homologous recombination deficiencies and a portion or the subjects in which cancer was present were positive for homologous recombination deficiencies. Subjects were considered positive for a homologous recombination deficiency when germline or somatic deletions including SNVs or indels in one of the following genes were detected: ATM, BRCA1, BRCA2, CDK12, CHEK2, PALB2, or RAD51D.

Counts of molecules included in the hyper partition for about 18,000 classification regions were determined. Normalized molecule counts for classification regions were determined based on molecule counts in control regions. 10-fold cross-fold validation was performed with samples randomly split with 1 group comprising 10% of the molecules used for testing and a subset of the other 90% of the molecules used for training

The training process for the computational model included selecting potential predictor regions by generating for each classification region a logistic regression model with the response variable being the HRD status for a given sample, and explanatory variables being the normalized counts for individual classification regions and cancer type for the individual samples. The top 300 regions based on p-values generated using the normalized classification region counts are selected. The normalized counts for the selected regions are used to generate a lasso logistic regression model to predict sample HRD status. Cross-validation is used to optimize lambda values and the computational model is fit to the training data. The computational model was generated using the glmnet-package in the R programming language with the final prediction scores being probit values.

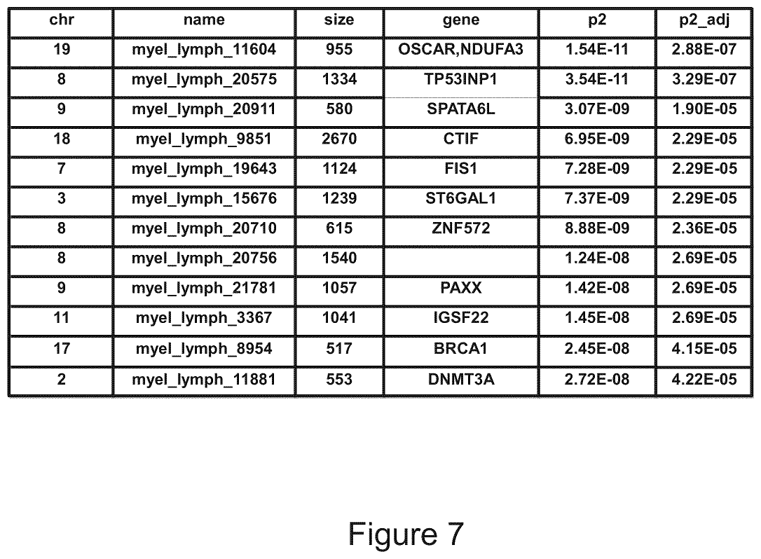

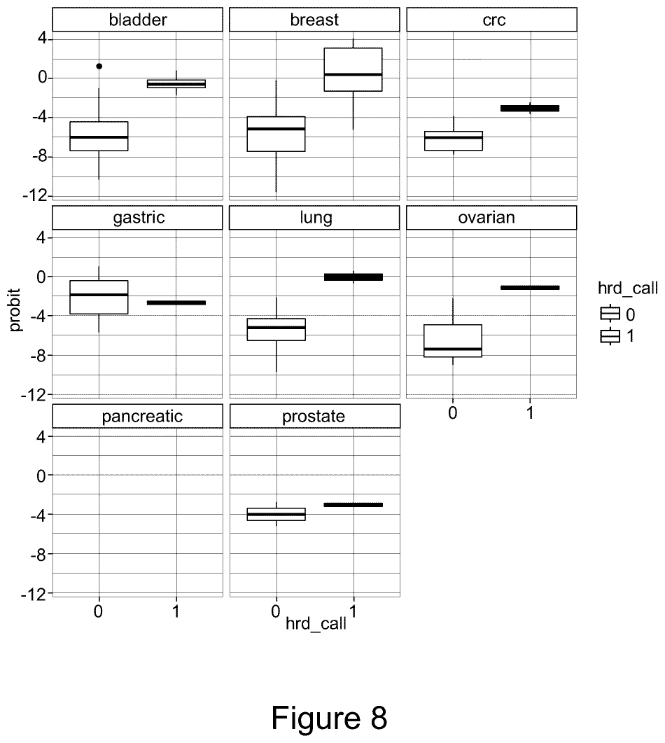

FIG. 7 is a chart showing genomic regions identified as predictor regions for homologous recombination repair deficiencies using techniques described herein. At least some of the genomic regions, such as ST6GAL1, PAXX, BRCA1, and DNMT3A are known to be involved in DNA repair mechanisms. The p2_adj column is an error rate adjustment for the p-values in the p2 column. FIG. 8 includes a number of graphs showing probit values related to determinations of subjects being positive for a homologous repair deficiency (HRD) or negative for an HRD using techniques described herein for a number of forms of cancer including bladder cancer, breast cancer, colorectal cancer, gastric cancer, lung cancer, ovarian cancer, pancreatic cancer, and prostate cancer. Separation between probit values for determining positive HRD status and negative HRD status is observed for several forms of cancer.

1. A method comprising:
obtaining, by a computing system having one or more hardware processors and memory, training sequence data including training sequence representations derived from a plurality of samples, individual training sequence representations including a nucleotide sequence corresponding to a fragment of a nucleic acid included in a sample of a plurality of samples and individual samples of the plurality of samples corresponding to a subject classified as having a homologous recombination repair deficiency;
determining, by the computing system, a subset of the training sequence representations that correspond to nucleic acids having at least a threshold amount of methylated cytosines in one or more regions of the nucleotide sequence;
analyzing, by the computing system, the subset of training sequence representations to determine quantitative measures derived from the subset of the training sequence representations, individual quantitative measures corresponding to a classification region of a plurality of classification regions of a reference genome, individual classification regions of the plurality of classification regions having the threshold amount of methylated cytosines in subjects in which cancer is detected;
analyzing, by the computing system and using one or more computational techniques, the quantitative measures of the plurality of classification regions to determine a subset of the plurality of classification regions having at least a threshold likelihood of indicating a homology directed repair deficiency; and
generating, by the computing system, a predictive model to determine a probability of a homologous recombination repair deficiency being present in one or more additional subjects, the predictive model including a plurality of variables and a plurality of weights with individual weights of the plurality of weights corresponding to individual variables of the plurality of variables, wherein an individual variable of the plurality of variables corresponds to an individual classification region of the subset of the plurality of classification regions and an individual weight that corresponds to the individual variable indicates a likelihood of the individual classification region indicating a homologous recombination repair deficiency.

2. The method of claim 1, comprising:
analyzing, by the computing system, the subset of training sequence representations to determine additional quantitative measures derived from the subset of the training sequence reads, individual quantitative measures corresponding to a control region of a plurality of control regions of a reference genome, individual control regions of the plurality of control regions having the threshold amount of methylated cytosines in subjects in which cancer is detected and in further subjects in which cancer is not detected; and
determining, by the computing system, normalized quantitative measures that correspond to the subset of the plurality of classification regions, wherein an individual normalized quantitative measure is determined according to the quantitative measure that corresponds to a classification region of the subset of the plurality of classification regions and the additional quantitative measures.

3. The method of claim 2, wherein determining the subset of the plurality of classification regions having at least a threshold likelihood of indicating a homology directed repair deficiency includes:
determining, by the computing system and for individual classification regions of the plurality of classification regions, differences between a first portion of the normalized quantitative measures derived from samples that correspond to subjects in which a homology directed repair deficiency is present and a second portion of the normalized quantitative measures derived from samples that correspond to additional subjects in which a homologous recombination repair deficiency is not present; and
determining, by the computing system, that an individual classification region is included in the subset of the plurality of classification regions based on the difference between the first portion of the normalized quantitative measures for the individual classification region and the second portion of the normalized quantitative measures for the individual classification region being at least a threshold difference.

4. The method of claim 1, comprising:
determining, by the computing system and implementing the predictive model, individual probabilities of a homologous recombination repair deficiency being present in individual samples of the plurality of samples based on the normalized quantitative measures corresponding to the individual samples; and
determining, by the computing system and based on the individual probabilities, a threshold probability to indicate a homologous recombination repair deficiency being present with respect to a given subject.

5. The method of claim 1, comprising:
determining, by the computing system, a responsiveness to treatment with respect to a group of subjects, wherein cancer is detected in the group of subjects and the treatment is provided to treat the cancer; and
determining, by the computing system, the plurality of samples that correspond to subjects having a homologous recombination repair deficiency based on the responsiveness of a portion of the group of subjects to the treatment being at least a threshold level of responsiveness.

6. The method of claim 5, wherein the treatment is a poly adenosine diphosphate (ADP) ribose polymerase (PARP) inhibitor.

7. The method of claim 1, comprising:
analyzing, by the computing system, additional sequence reads derived from samples of a group of subjects in which cancer is detected to determine whether one or more genomic mutations are present with respect to one or more genomic regions, wherein the one or more genomic mutations correspond to homologous recombination repair pathways; and
determining, by the computing system, the plurality of samples used to produce the training sequence representations by identifying a portion of the samples derived from the group of subjects in which the one or more genomic mutations are present.

8. The method of claim 1, wherein the one or more computational techniques include implementing one or more logistic regression models with elastic regularization.

9. The method of claim 1, comprising:
implementing, by the computing system, the predictive model to determine a probability of a homologous recombination repair deficiency being present in a plurality of additional samples, the plurality of additional samples being derived from additional subjects with a first form of cancer being detected in a first portion of the additional subjects and a second form of cancer being detected in a second portion of the additional subjects.

10. The method of claim 1, comprising:
implementing, by the computing system, the predictive model to determine a probability of a homologous recombination repair deficiency being present in a plurality of additional samples, the plurality of additional samples being derived from additional subjects in which a single form of cancer is present.

11. The method of claim 1, comprising:
analyzing, by the computing system, the subset of training sequence reads to determine a group of training sequence reads that correspond to a plurality of genomic regions associated with homologous recombination repair pathways; and
determining, by the computing system, one or more additional quantitative measures based on a number of the group of training sequence representations that correspond to at least a portion of the plurality of genomic regions.

12. The method of claim 11, comprising:
determining, by the computing system, an additional subset of the training sequence representations that correspond to additional nucleic acids having less than an additional threshold amount of methylation;
analyzing, by the computing system, the additional subset of the training sequence reads to determine an additional group of training sequence representations that correspond to the plurality of genomic regions associated with the homologous recombination repair pathways;
determining, by the computing system, one or more further quantitative measures based on an additional number of the additional group of training sequence representations that correspond to at least a portion of the plurality of genomic regions.

13. The method of claim 12, comprising:
analyzing, by the computing system, differences between the one or more additional quantitative measures and the one or more further quantitative measures to determine one or more additional variables for the predictive model.

14. The method of claim 1, wherein the plurality of classification regions have at least a threshold amount of cytosine-guanine content.

15. The method of claim 1, comprising:
determining, by the computing system, tumor fraction estimates for a number of samples, the number of samples corresponding to subjects in which cancer is detected;
analyzing, by the computing system, the tumor fraction estimates with respect to a threshold tumor fraction estimate; and
determining, by the computing system, the plurality of samples used to derive the training sequence reads based on identifying at least a portion of the number of samples having a tumor fraction estimate corresponding to at least the threshold tumor fraction estimate.

16. The method of claim 1, comprising:
obtaining, by the computing system, testing sequence data from an additional subject that is not included in the plurality of subjects, the testing sequence data including testing sequencing representations derived from a sample of the additional subject, individual testing sequencing representations including a nucleotide sequence corresponding to a fragment of a nucleic acid included in the additional sample and individual testing sequencing reads corresponding to molecules having the threshold amount of methylated cytosines included in regions of the nucleotide; and
determining, using the predictive model and the additional sequence data, a probability of a homologous recombination repair deficiency being present in the additional subject.

17. The method of claim 16, comprising:
analyzing, by the computing system, the testing sequencing reads to determine first additional quantitative measures that correspond to the individual classification regions of the plurality of classification regions;
analyzing, by the computing system, the testing sequencing reads to determine second additional quantitative measures derived from the testing sequencing reads that correspond to individual control regions of a plurality of control regions, the individual control regions of the plurality of control regions having the threshold amount of methylated cytosines in subjects in which cancer is detected and in further subjects in which cancer is not detected;
determining, by the computing system, additional normalized quantitative measures that correspond to the subset of the plurality of classification regions, wherein an individual additional normalized quantitative measure is determined according to the first additional quantitative measures and the second additional quantitative measures; and
generating, by the computing system, an input vector that includes the normalized quantitative measures;
wherein the predictive model uses the input vector to determine the probability of a homologous recombination repair deficiency being present in the additional subject.

18. The method of claim 1, comprising:
combining a plurality of nucleic acids derived from at least one of blood or tissue of a subject with a solution including an amount of methyl binding domain (MBD) proteins to produce a nucleic acid-MBD protein solution; and
performing a plurality of washes of the nucleic acid-MBD protein solution with a salt solution to produce a number of nucleic acid fractions, individual nucleic acid fractions having a threshold number of methylated cytosines in regions of the plurality of nucleic acids having at least a threshold cytosine-guanine content.

19. The method of claim 18, wherein a wash of the plurality of washes is performed with a solution having a concentration of sodium chloride (NaCl) and produces a nucleic acid fraction of the number of nucleic acid fractions having a range of binding energies to MBD proteins.

20. The method of claim 18, comprising:
determining that a first nucleic acid fraction is associated with a first partition of a plurality of partitions of nucleic acids, the first partition corresponding to a first range of binding energies to MBD proteins;
causing a first molecular barcode to attach to nucleic acids of the first nucleic acid fraction, the first molecular barcode being associated with the first partition;
determining that a second nucleic acid fraction is associated with a second partition of the plurality of partitions of nucleic acids, the second partition corresponding to a second range of binding energies to MBD proteins different from the first range of binding energies to MBD proteins; and
causing a second molecular barcode to attach to nucleic acids of the second nucleic acid fraction, the second molecular barcode being associated with the second partition.

21. The method of claim 18, comprising:
combining at least a portion of the number of nucleic acid fractions with an amount of restriction enzyme that cleaves molecules with one or more unmethylated cytosines to produce at least a portion of the plurality of samples used to produce the training sequence representations;
wherein the threshold amount of methylated cytosines corresponds to a minimum frequency of methylated cytosines within a region having at least the threshold cytosine-guanine content.

22. The method of claim 18, comprising:
combining at least a portion of the number of nucleic acid fractions with an amount of a restriction enzyme that cleaves molecules with one or more methylated cytosines to produce at least a portion of the plurality of samples used to produce the training sequence representations;
wherein the threshold amount of unmethylated cytosines corresponds to a maximum frequency of methylated cytosines that are not cleaved within a region having at least the threshold cytosine-guanine content.

23. The method of claim 1, comprising
administering treatment to the subject based on a determination of a homologous recombination repair deficiency being present in the subject.
